SEQUENCE LISTING (1 sequence)

SEQ ID NO: 1 - DNA, 17 bp, synthetic construct
  source 1..17: mol_type = other DNA
cggcatcgtacgattat

(19) United States
(12) Patent Application Publication
(10) Pub. No.: US 2024/0199681 A1
(43) Pub. Date: Jun. 20, 2024

(54) OLIGONUCLEOTIDE AND NUCLEIC ACID SYNTHESIS

(71) Applicant: EVONETIX LTD, Cambridge (GB)

(72) Inventors: Stuart Crosby, Essex (GB); Matthew Jenison, Cambridge (GB); Joseph Brennam, Cambridge (GB); Iain Barlow, Cambridge (GB); Pascal Lang, Cambridge (GB); Matthew Hayes, Cambridge (GB); Catherine Fitzpatrick, Cambridge (GB); Daniel Bygrave, Cambridge (GB)

(21) Appl. No.: 18/353,269

(22) Filed: Jul. 17, 2023

Related U.S. Application Data
(62) Division of application No. 16/964,390, filed on Jul. 23, 2020, now abandoned.

(30) Foreign Application Priority Data
Jan. 24, 2018 (GB) 1801182.5

(51) Int. Cl. C07H 21/04; C07H 1/00; C12N 15/10; C12Q 1/6811

(52) U.S. Cl. CPC C07H 21/04; C07H 1/00; C12N 15/1006; C12Q 1/6811

(57) ABSTRACT

The present invention relates to methods for the high fidelity synthesis of oligonucleotides and polynucleotides on a solid surface. In particular, the invention relates to methods of synthesising oligonucleotides, polynucleotides, and doublestranded polynucleotides/nucleic acids, such as DNA and XNA, wherein the process comprises thermally controlled deprotection steps at the 5′-OH of previously coupled nucleosides or nucleotides at selected sites on the surface of the substrate.

### SEQUENCE LISTING

The instant application contains a Sequence Listing which has been submitted electronically in XML format and is hereby incorporated by reference in its entirety. Said XML copy, created on Jan. 26, 2024, is named 101705000013_Sequence Listing and is 1,979 bytes in size.

### CROSS-REFERENCE TO RELATED APPLICATIONS

This application is a divisional of U.S. patent application Ser. No. 16/964,390 filed Jan. 23, 2019, which is the National Stage of International Patent Application No. PCT/GB2019/050192, filed Jan. 23, 2019, which claims the benefit of Great Britain Patent Application No. 1801182.5, filed Jan. 24, 2018, the disclosures of which are incorporated herein by reference in their entireties.

### FIELD OF THE INVENTION

The present invention relates to methods for synthesising oligonucleotides and polynucleotides. In particular, the invention relates to methods of synthesising oligonucleotides, polynucleotides, and double-stranded polynucleotides, such as DNA and XNA.

### BACKGROUND OF THE INVENTION

There is an increasing demand for artificial or synthetic synthesis of polynucleotides. Using readily available techniques of molecular biology, it is possible to replicate and amplify polynucleotides from natural sources. Such techniques additionally enable engineering of natural nucleic acid sequences, for example through substitution, insertion or deletion of one or more nucleotides, and hence provide access to nucleic acid sequences that are not naturally available.

However, such approaches are often time-consuming and labour intensive. In addition, a reliance on natural sequences as a starting point may limit the scope of sequences that are practically achievable. Furthermore, difficulty in accessing natural polynucleotides themselves may give rise to additional obstacles.

De novo synthesis of polynucleotides offers a route to theoretically any nucleic acid sequence and may therefore overcome some of the issues with traditional molecular biology-based approaches.

In vitro synthesis of relatively short oligonucleotides, for example via solid-phase synthesis using the phosphoramidite method, is well known. Indeed, traditional molecular biology often relies on synthesised oligonucleotide primers for use in polymerase chain reaction (PCR) and site-directed mutagenesis methods.

Polynucleotides may be synthesised by connecting a number of separately synthesised oligonucleotides. Typically under this approach, a group of oligonucleotides is synthesised, for example using automated solid-phase synthesis, and purified, then the individual oligonucleotides are subsequently connected together by annealing and ligation or polymerase reactions.

However, typical automated oligonucleotide synthesis techniques via chemical reactions generate a random base errors in the oligonucleotides, due to unintended side reactions or missed reactions. For example, a coupling failure results when an oligonucleotide does not react with the next nucleoside building block but retains the reactive 5′-OH, which then participates in the next coupling round, resulting in an oligonucleotide with a missing base (a deletion error). Over each successive cycle the deletion errors accumulate, resulting in a final product containing a complex mixture of oligonucleotides that would be extremely difficult to purity. In order to address the deletion error, typically, the phosphoramidite method includes a “capping” step in the cycle, whereby the coupling failures are removed from further participation in the synthesis. This is typically achieved by acetylation of the unreacted 5′—OH groups with acetic anhydride and N-methyl imidazole. This reagent reacts only with free hydroxyl groups to irreversibly cap the oligonucleotides in which coupling failed.

Typical oligonucleotide synthesis techniques do not give rise to 100% yields for each nucleotide addition step. Even with a yield of 99.5% per coupling round, the yields multiply over the length of a nucleic acid sequence and lead to significant difficulties in the provision of longer polynucleotides, such as full length genes and genomes, resulting in very low overall yields, and a waste of starting materials and intermediates, and the possibility of forming oligonucleotide mixtures at the end.

Accordingly, there remains a significant need in the art for methods of accurately and efficiently providing polynucleotides, in particular those of whole gene and genome scale.

Therefore, there is an urgent need for new technology to produce high-fidelity polynucleotides.

### SUMMARY OF THE INVENTION

In its broadest aspect, the present invention relates process for the parallel synthesis of a plurality of oligonucleotides, such as DNA and XNA, on a surface of a solid substrate, wherein deprotection of the 5′-OH protecting group is carried out under thermal control at selected sites of the solid substrate, thereby allowing coupling of a 5′-OH protected nucleoside or a 5′-OH protected nucleotide phosphoramidite building block to the deprotected 5′-OH at those sites. The process of thermally controlling the deprotection at selected sites followed by coupling of a 5′-OH protected nucleoside or a 5′-OH protected nucleotide phosphoramidite to the free 5′—OH group is repeated until the desired oligonucleotides are formed at each site of the solid substrate. The present invention therefore provides a massively parallel synthesis of oligonucleotides, wherein the coupling of a 5′-OH protected nucleoside or a 5′-OH protected nucleotide phosphoramidite building block to build the oligonucleotide is controlled by selectively deprotecting the growing end of nucleoside/oligonucleotide.

The present invention additionally relates to a process for the parallel synthesis of oligonucleotides on a plurality of sites on the surface of a solid substrate, said oligonucleotides being the same or different wherein the process comprises providing each site with a plurality of nucleosides (or nucleotides such as a di- or tri-nucleotide) comprising a 5′-OH thermally cleavable protecting group, wherein the nucleosides or nucleotides are immobilized on the surface of a solid substrate; and wherein the process involves deprotecting the 5′—OH groups at selected sites, and coupling each free 5′—OH groups at the sites with a nucleoside containing a 5′-OH-thermally cleavable protecting group or a nucleotide containing a 5′-OH thermally cleavable protecting group. Preferably, the process comprises providing each site with a plurality of nucleosides comprising a 5′-OH thermally cleavable protecting group, wherein the nucleosides are immobilized on the surface of a solid substrate; and wherein the process involves deprotecting the 5′—OH groups at selected sites, and coupling each free 5′—OH groups at the sites with a nucleoside phosphoramidite containing a 5′-OH thermally cleavable protecting group, or a nucleotide phosphoramidite containing a 5′-OH thermally cleavable protecting group (preferably a di- or tri-nucleotide 3′-phosphoramidite containing a 5′-OH-thermally cleavable protecting group). The process of selective, thermally controlled 5′-OH deprotection, and reacting with another nucleoside (e.g. a nucleoside 3′-phosphoramidite, or a nucleotide phosphoramidite such as a di- or tri-nucleotide 3′-phosphoramidite) containing a 5′-OH-thermally cleavable protecting group, is repeated in order to produce the desired oligonucleotide sequences at each site.

In any aspect of the present invention, the selective deprotection of the thermally cleavable protecting groups can be achieved by application of heat at the selected sites of the substrate, e.g. in the presence of a solvent. Preferably, the selective deprotection does not require an additional reagent.

In any aspect of the present invention, the selective cleavage of the thermally cleavable linker can be achieved by application of heat at the selected sites of the substrate, e.g in the presence of a solvent. Preferably, the selective cleavage does not require an additional reagent.

The present invention also relates to a process of parallel oligonucleotide synthesis that uses a substrate (e.g. a flow cell) containing a plurality of thermally addressable reaction sites, wherein individual oligonucleotide components can be grown in a thermally controlled way, wherein the thermally controlled growth involves selective, thermally controlled deprotection of the 5′-OH protected nucleoside building block, at specific reaction sites, to allow coupling of a 5′-OH protected nucleoside (or a 5′-OH protected di- or tri-nucleotide 3′-phosphoramidite containing a 5′-OH-thermally cleavable protecting group building block at the deprotected 5′-OH. The selective, thermally controlled deprotection and coupling steps are repeated until the desired oligonucleotide sequences are produced at each site, to form an oligonucleotide microarray.

The thermally addressable reaction sites provide highly controlled, localised areas of heat which enables selective deprotection of the 5′-OH protected nucleoside (or 5′-OH protected nucleotide) building block at the selected sites, to allow coupling of the next 5′-OH protected nucleoside (or 5′-OH protected nucleotide) building block.

The thermally controlled deprotection is achieved by the provision of a thermally cleavable protecting group at the 5′—OH group of each of the nucleoside (or nucleotide) building blocks.

Advantageously, the starting nucleoside (or nucleotide) is bound to the substrate by a thermally cleavable linker group. This thermally cleavable linker group is preferably protected (i.e. a safety catch linker group) so that it is only removed at the end of the oligonucleotide synthesis. Advantageously, the thermally cleavable linker enables the oligonucleotides to be released selectively under thermal control, in order to enable selective, highly controlled hybridisation of the oligonucleotides to form double stranded nucleic acids or nucleic acid fragments.

Another aspect of the present invention encompasses a process for the parallel synthesis of one or more oligonucleotides on a plurality of sites on the surface of a solid substrate, said oligonucleotides being the same or different wherein the process comprises:
(i) providing each site with a plurality of nucleosides, or nucleotides (preferably di-nucleotides, or tri-nucleotides) comprising a 5′-OH protecting group, wherein the nucleosides, or nucleotides are immobilized on the surface of a solid substrate;
(ii) conducting thermally controlled deprotection at the 5′-OH of the nucleosides or nucleotides at selected sites on the surface of the solid substrate to form, at each of the selected sites, nucleosides (or nucleotides) having deprotected 5′-OH groups;
(iii) at each of the selected sites, coupling onto the deprotected 5′—OH groups, a nucleoside 3′-phosphoramidite (or a di-nucleotide 3′-phosphoramidite, or a tri-nucleotide 3′-phosphoramidite) comprising a 5′-OH protecting group, and oxidising the resulting phosphite triester group to a phosphate triester group;
(iv) conducting thermally controlled deprotection at the 5′-OH of the nucleosides or nucleotides at selected sites on the surface of the substrate, wherein the selected sites can be the same as, or different from, the selected sites of the preceding step,
(v) at each of the selected sites, coupling onto the deprotected 5′—OH groups, a nucleoside 3-phosphoramidite (or a di-nucleotide 3-phosphoramidite, or a tri-nucleotide 3-phosphoramidite) comprising a 5′-OH protecting group, and oxidising the resulting phosphite triester group to a phosphate triester group; and
(vi) repeating steps (iv) and (v) one or more times to obtain the desired oligonucleotides at each site on the surface of a solid substrate.

Preferably, step (i) above comprises providing each site with a plurality of nucleosides comprising a 5′-OH protecting group, wherein the nucleosides are immobilized on the surface of a solid substrate.

Yet another aspect of the present invention provides a process for the parallel synthesis of one or more oligonucleotides on a plurality of sites on the surface of a chip, said oligonucleotides being the same or different wherein the process comprises:
(i) providing each site with a plurality of nucleosides, or nucleotides (preferably wherein the nucleotides are di-nucleotides, or tri-nucleotides) comprising a 5′-OH thermally cleavable protecting group, wherein the nucleosides are attached to the surface of a solid substrate at the 3-position via a thermally cleavable linker group:
(ii) thermally controlled deprotection at the 5′-OH of the nucleosides at selected sites on the surface of the chip to form, at each of the selected sites, nucleosides or nucleotides) having deprotected 5′—OH groups;
(iii) at each of the selected sites, coupling onto the deprotected 5′—OH groups, a nucleoside 3-phosphoramidite (or a di-nucleotide 3-phosphoramidite, or a tri-nucleotide 3-phosphoramidite) comprising a thermally cleavable 5′-OH protecting group, and oxidising the resulting phosphite triester group to a phosphate triester group;
(iv) thermally controlled deprotection at the 5′-OH of the nucleosides or nucleotides at selected sites on the surface of the substrate, wherein the selected sites can be the same as, or different from, the selected sites of the preceding step,
(v) at each of the selected sites, coupling onto the deprotected 5′—OH groups, a nucleoside 3′-phosphoramidite (or a di-nucleotide 3′-phosphoramidite, or a tri-nucleotide 3′-phosphoramidite) comprising a thermally cleavable 5′-OH protecting group, and oxidising the resulting phosphite triester group to a phosphate triester group;
(vi) repeating steps (iv) and (v) one or more times to obtain the desired oligonucleotides at each site on the surface of the chip, wherein the chip comprises individually thermally addressable sites.

Preferably, step (i) above comprises providing each site with a plurality of nucleosides comprising a 5′-OH protecting group, wherein the nucleosides are immobilized on the surface of a solid substrate.

The processes of the present invention enables massively parallel synthesis of a plurality of different oligonucleotides on the surface of the substrate, by utilising thermally controlled deprotection of selected nucleosides or nucleotides, thereby enabling selective reaction of those nucleosides or nucleotides with an incoming nucleoside or nucleotide building block. Since each site is independently thermally addressable, only the nucleosides or nucleotides at the sites which are heated are deprotected, and hence available for reaction in the coupling step. Moreover, since the first nucleoside or nucleotide is bound to the surface of the substrate, the reagents can be washed over the solid substrate, such that the coupling reaction only occurs on the sites that were heated and thus have a deprotected 5′—OH group leaving the other sites unaffected. The process allows for the high fidelity, parallel synthesis of the desired oligonucleotides at each site.

The invention further provides a microarray comprising one or more nucleotides, oligonucleotides or nucleic acids on a plurality of sites on the surface of a solid substrate, wherein the nucleotides, oligonucleotides or double stranded nucleic acids are bound to the surface by a thermally cleavable linker.

In a further aspect the invention provides a microarray which is preparable or obtainable by any of the processes described herein.

Yet another aspect of the invention provides the use of a process as described in any aspect or embodiment herein, or the use of a microarray as described in any aspect or embodiment disclosed herein for preparing an oligonucleotide, a nucleic acid, preferably DNA or XNA.

The invention additionally provides an oligonucleotide or nucleic acid, which is preparable or obtainable by any of the processes described herein.

The process can additionally further comprise thermally controlled release of the resulting oligonucleotides at selected sites, wherein the selectively released oligonucleotides hybridise with selected immobilized oligonucleotides under thermal control to form nucleic acids.

The process can be further combined with error-detection operations in the hybridisation steps in order to further increase the purity of the final nucleic acid.

### DESCRIPTION OF THE FIGURES

FIG. 1: Time course study results for cleavage of deprotected linker of Example 1C at 90° C. and at 20° C.

FIG. 2: Time course study results for cleavage of deprotected linker of Example 1C using different solvent systems at pH 7.4 PBS and acetonitrile and pH 5 buffer (TEEA)

FIG. 3: Time course study results for cleavage of deprotected linker of Example 1C using different ratios of PBS:MeCN (acetonitrile) at 90° C.

FIG. 4A: Time course study results for deprotection (removal of Bsmoc) and cleavage of Bsmoc protected linker of Example 2 at 90° C.

FIG. 4B: Time course study results for deprotection of Bsmoc-protected linker (i.e. removal of Bsmoc) of Example 2 at room temperature and 90° C.

FIG. 4C: Time course study results showing cleavage of Bsmoc protected linker of Example 2 to give free TBDPS-thymidine at 90° C. and at 20° C. (formation and cleavage of deprotected intermediate not shown)

FIG. 5: Stability study results for the Bsmoc protected linker of Example 2 under different pH conditions at 80° C.

FIG. 6: Stability study results for deprotected linker of Example 3 under different temperature conditions (room temperature vs 90° C.)

FIG. 7: Stability study results for Fmoc-protected linker of Example 3 using 10% diisopropylamine under different temperature conditions (room temperature vs 90° C.

FIG. 8: Stability study results for Fmoc-protected linker of Example 3 using 10% diisopropylamine at 90° C. using different solvents (DMF vs acetonitrile)

FIG. 9: Stability study results for Fmoc-protected linker of Example 3 using 20% diisopropylamine in 2:1 DMF (dimethylformamide):CAPs (N-cyclohexyl-3-aminopropanesulfonic acid) buffer at different temperatures (10° C. vs 90° C.)

FIG. 10: Cleavage of deprotected linker of Example 1C and Example 4C at 90° C.

FIG. 11: Stability study results (Example 5F) for the Compound of Example 4C at 90° C. in different solvent systems

FIG. 12: Time course study results (Example 5G) for deprotection of Boc-protected linker (Compound of Example 1B)

FIG. 13: Time-course study on the non-protected α-Phenyl Safety-Catch Linker (compound of Example 6D)

FIG. 14: Time-course study on cleavage of non-protected Double Safety-Catch Linker (Compound of Example 8C) vs Single Safety-Catch Linker (Compound of Example 1C)

FIG. 15: Time-course study on the non-protected 5′-linked protected 3′ O-Acetyl-Thymidine (Compound of Example 13B)

FIG. 16: Schematic illustration of an example of a temperature control device for controlling temperatures at respective sites within a medium.

FIG. 17: Top view of the temperature control device.

FIG. 18: Cross-section through the temperature control device in more detail.

FIG. 19: Graph showing an example of changes in temperature in a fluid as it flows over active thermal sites and passive thermal regions of the temperature control device.

FIG. 20: Illustration of a thermal model for an active thermal site.

FIG. 21: Illustration of a first order approximation of the system as an active thermal site surrounded by four passive thermal regions.

FIG. 22: Electrical circuit model analogous to the thermal model.

FIG. 23: Compacted version of the model of FIG. 22.

FIG. 24: Plot showing how the heat supplied to the medium varies with the heat generated by the heating element.

FIG. 25: Illustration of a feedback loop architecture for controlling the temperature at a given active site.

FIG. 26: Flow diagram illustrating a method of controlling temperatures at respective sites in a medium.

FIG. 27: Illustrative examples of a pillared structure for the thermal insulation layer of an active site.

FIG. 28: Cross-section through two active sites and several passive sites, where the thermal insulation layer has a pillared structure including voids.

FIG. 29: Flow diagram illustrating a method of manufacturing a temperature control device with a pillared thermal insulation layer.

FIG. 30: Illustration of the respective stages of the manufacturing method of FIG. 29.

### DETAILED DESCRIPTION OF THE INVENTION

### Definitions

The terms used herein have their normal meanings in the art unless otherwise indicated.

The term “nucleotide” refers to a nucleic acid (preferably DNA or analogues thereof) subunit that includes a sugar group, a heterocyclic base and a phosphate group.

The term “nucleoside” refers to a compound comprising a sugar group covalently coupled to a heterocyclic base. The heterocyclic base of a nucleoside or nucleotide is also known as a nucleobase. Nucleotides each comprise a nucleobase. The term “nucleobase” or “base” as used herein refers to nitrogenous bases, including purines and pyrimidines, such as the DNA nucleobases A, T, G and C, the RNA nucleobases A, U, C and G, as well as non-DNA/RNA nucleobases, such as 5-methylcytosine (^{Me}C), isocytosine, pseudoisocytosine, 5-bromouracil, 5-propynyluracil, 5-propyny-6-fluorouracil, 5-methylthiazoleuracil, 6-aminopurine, 2-aminopurine, inosine, 2,6-diaminopurine, 7-propyne-7-deazaadenine, 7-propyne-7-deazaguanine and 2-chloro-6-aminopurine.

Nucleic acids may be, for example, single- or double-stranded.

Xeno nucleic acid (XNA) is a synthetic nucleic acid that is an artificial alternative to DNA. As with DNA, XNA is an information-storing polymer, however XNA differs from DNA and RNA in the structure of the sugar-phosphate backbone. By 2011, at least six synthetic sugars had been used to create XNA backbones that are capable of storing and retrieving genetic information. Substitution of the backbone sugars makes XNAs functionally and structurally analogous to DNA.

The term “hybridisation” refers to the hydrogen bonding of opposing nucleic acid strands, preferably Watson-Crick hydrogen bonding between complementary nucleoside or nucleotide bases.

In all cases, unless otherwise indicated, references to nucleoside(s), nucleotide(s) and oligonucleotide(s) include those having activating or protecting groups as appropriate.

In all cases, unless otherwise indicated, references to nucleoside(s), nucleotide(s) and oligonucleotide(s) include naturally occurring purine and pyrimidine bases, in particular, adenine, thymine, cytosine, guanine and uracil, as well as modified purine and pyrimidine analogues, such as alkylated, acylated, or protected purines and pyrimidines. Hence, the nucleoside(s), nucleotide(s) and oligonucleotide(s) can include optionally protected canonical or optionally protected non-canonical nucleobases.

In all cases, unless otherwise indicated, the terms “oligonucleotide” and “polynucleotide” are used interchangeably, and refer to naturally occurring, as well as synthetic, polymers formed from nucleotides. These may be single- or double-stranded.

The term “hydrocarbyl” as used in herein refers to a monovalent group formed by removing a hydrogen atom from a hydrocarbon. The term hydrocarbyl encompasses alkyl, aryl, alkaryl and arylalkyl, alkenyl, or alkynyl groups as defined below. The alkyl groups and alkyl portions of the hydrocarbyl groups can include straight chain, branched, or cyclic alkyl.

Alkyl groups relates to saturated, straight, branched, primary, secondary or tertiary or cyclic hydrocarbons. Alkyl groups can contain from 1-20 carbon atoms, 1-15 carbon atoms, or 1-6 carbon atoms. Particularly preferred alkyl groups are C₁₋₆ straight or branched alkyl groups, or C₃₋₆ cycloalkyl groups. Preferred alkyl groups are methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. More preferably alkyl groups can contain from 1-6 carbon atoms, particularly methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Alkyl also encompasses cycloalkyl groups which can contain 3 to 10 carbon atoms having single or multiple fused rings. Preferred cycloalkyl groups include adamantyl, cyclopropyl, cyclobutyl, cyclopentyl, cyclohexyl, cycloheptyl, cyclooctyl, cyclopropyl, cyclobutyl, cyclopentyl or hexyl.

Aryl groups relate to aromatic rings containing from 6-20, preferably 6-15 and more preferably 6-10 carbon atoms, and include monocyclic, bicyclic and polycyclic, fused or branched aryl groups. Preferred aryl groups are phenyl, biphenyl and naphthyl. A particularly preferred aryl group is phenyl.

Alkaryl groups can contain from 7-21 carbon atoms, preferably 7-16 carbon atoms and more preferably 7-11 carbon atoms, and include alkaryl group containing monocyclic, bicyclic and polycyclic or branched aryl groups, as well as straight, branched or cyclic alkyl groups. Preferred alkaryl groups are tolyl and xylyl.

Arylalkyl groups can contain from 7-21 carbon atoms, preferably 7-16 carbon atoms and more preferably 7-11 carbon atoms, and include arylalkyl groups containing monocyclic, bicyclic and polycyclic or branched aryl groups, as well as straight, branched or cyclic alkyl groups. Preferred arylalkyl groups are benzyl, phenethyl, phenpropyl, phenbutyl, naphthylmethyl and naphthylmethyl.

Alkenyl refers to straight, branched and cyclic hydrocarbons having at least one carbon-carbon double bond. Preferably, alkenyl groups contain from 2-12, 2-8, 2-6 or 2-4 carbon atoms. Preferably, alkenyl refers 1-3 double bonds, and more preferably one double bond. Alkenyl groups preferably include: ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl, and cyclopenten-4-yl.

Alkynyl relates to straight, branched or cyclic hydrocarbons having at least one carbon-carbon triple bond, preferably having 1-2 triple bonds, and more preferably one triple bond. Preferably, alkynyl groups include from 2 to 12 carbon atoms, preferably 2-8 or more preferably 2-4 carbon atoms. Preferred alkynyl groups are: ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl and 4-methylpent-2-yn-5-yl.

In any aspect or embodiment of the present invention, hydrocarbyl preferably refers to alkyl, aryl or arylalkyl, more preferably C₁₋₆ alkyl, C₆₋₁₀ aryl or C₇₋₁₂ arylalkyl. Even more preferably, hydrocarbyl refers to C₆₋₁₀ aryl, or C₇₋₁₂ arylalkyl, and most preferably phenyl or benzyl.

A heterocyclic group, for example, in the context of the ring A, refers to a non-aromatic cyclic group containing at least one ring nitrogen atom, i.e. the nitrogen atom which is part of the

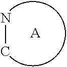

moiety. The ring A heterocyclic group, which is represented by:

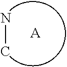

may be monocyclic, bicyclic or tricyclic, and preferably is monocyclic or bicyclic, more preferably monocyclic.

The heterocyclic group may contain unsaturated ring carbon atoms, but is preferably saturated. Preferably, the heterocyclic group of ring A is a 4-12 membered heterocyclic ring containing at least one ring nitrogen atom.

Suitable ring A heterocyclic groups for include: azetidinyl, pyrrolidinyl, 2,5-dihydropyrrole, pyrazolinyl, imidazolyl, imidazolinyl, oxazolidinyl, thiazolidinyl, isothiazolyl, isothiazolidinyl, piperidinyl, piperazinyl, 2-oxopiperazinyl, 2-oxopiperidinyl, 2-oxoazepinyl, azepinyl, 4-piperidonyl, morpholinyl, thiamorpholinyl and triazolyl. Bicyclic heterocyclic groups include, but are not limited to tetra-hydroisoquinolinyl and tetrahydroquinolinyl Preferred heterocyclic groups are those at least one ring nitrogen atom, and most preferably a 5 or 6-membered heterocyclic ring containing one ring nitrogen atom. Particularly, the ring A is a heterocyclic group selected from the group consisting of piperidinyl, pyrrolidinyl, azepanyl (homopiperidinyl) and azocanyl, and more preferably ring A is a heterocyclic group selected from the group consisting of piperidinyl, pyrrolidinyl and azepanyl, and most preferably ring A is piperidinyl or pyrrolidinyl. The ring A heterocyclic group can be unsubstituted or substituted at one or more ring atoms (preferably with inert substituents, such as alkyl, aryl, aryalkyl, or alkyaryl). Thus references to ring A and specific ring A groups include those having substituents on one or more ring atoms. Preferably, Ring A is unsubstituted.

The term “protecting group” refers to a moiety which is used to temporarily mask a reactive group on a molecule, in order to enable chemical transformation on another part of the molecule, and which can be subsequently removed. Protecting groups for different functional groups and reaction conditions are well known, e.g. from Greene's “Protective Groups in Organic Synthesis”, Fifth edition (2014), Peter G. M. Wuts, Wiley.

The term “thermally cleavable” as used in the context of linker groups or protecting groups means that the linker group or protecting group is readily susceptible to cleavage by the application of heat, preferably in the presence of a solvent.

The terms “fragment”, “moiety”, “group”, “substituent” and “radical” are used herein interchangeably to refer to a portion of a molecule, for example having a particular functional group.

It will be appreciated that certain compounds of the present invention may contain one or more chiral centres. Unless otherwise indicated, references to a compound of unspecified stereochemistry are intended to include the single isomers or single enantiomers, or mixtures including racemates thereof.

Aspects of the invention relate to processes for preparing DNA or XNA, preferably DNA or XNA. However, the technology could readily be applied to the preparation of other polynucleotides.

An aspect of the present invention provides a process for the parallel synthesis of one or more oligonucleotides (e.g. DNA, or XNA) on a plurality of sites on the surface of a solid substrate, said oligonucleotides being the same or different wherein the process comprises:
(i) providing each site with a plurality of nucleosides or nucleotides (preferably wherein the nucleotides are di-nucleotides, or tri-nucleotides), comprising a 5′-OH protecting group, wherein the nucleosides or nucleotides are immobilized on the surface of a solid substrate;
(ii) conducting thermally controlled deprotection at the 5′-OH of the nucleosides or nucleotides at selected sites on the surface of the solid substrate to form, at each of the selected sites, nucleosides having deprotected 5′—OH groups;
(iii) at each of the selected sites, coupling onto the deprotected 5′—OH groups, a nucleoside 3′-phosphoramidite, or a nucleotide 3′-phosphoramidite (preferably wherein the nucleotide 3′-phosphoramidite is a di-nucleotide 3′-phosphoramidite, or a tri-nucleotide 3′-phosphoramidite), wherein the nucleoside 3′-phosphoramidite, or nucleotide 3′-phosphoramidite comprises a 5′-OH protecting group, and oxidising the resulting phosphite triester group to a phosphate triester group;
(iv) conducting thermally controlled deprotection at the 5′-OH of the nucleosides or nucleotides at selected sites on the surface of the substrate, wherein the selected sites can be the same as, or different from, the selected sites of the preceding step,
(v) at each of the selected sites, coupling onto the deprotected 5′—OH groups, a nucleoside 3′-phosphoramidite, or a nucleotide 3′-phosphoramidite (preferably wherein the nucleotide 3′-phosphoramidite is a di-nucleotide 3′-phosphoramidite, or a tri-nucleotide 3′-phosphoramidite), wherein the nucleoside 3′-phosphoramidite, or nucleotide 3′-phosphoramidite comprises a 5′-OH protecting group, and oxidising the resulting phosphite triester group to a phosphate triester group; and
(vi) repeating steps (iv) and (v) one or more times to obtain the desired oligonucleotides at each site on the surface of a solid substrate.

In step (i) the surface of the solid substrate is provided with nucleosides or nucleotides (preferably di- or tri-nucleotides) (“starting nucleosides” or “starting nucleotides”) which are bound to the surface to form a “reaction site”. Within a single reaction site there may be a plurality of the same starting nucleoside each bound to the surface of the substrate. Different reaction sites may comprise different surface-bound nucleosides or nucleotides, depending on the desired oligonucleotides to be synthesised. Since the reaction sites are under independent thermal control, each reaction site may be used to synthesise different oligonucleotides independently from the other reaction sites. Preferably, in step (i), the surface of the solid substrate is provided with nucleosides (“starting nucleosides”).

The 5′-OH-protected nucleosides or nucleotides of step (i) preferably comprise a thermally cleavable 5′-OH protecting group. The thermally protecting group may be of the safety catch type, whereby two, separate steps (activation and cleavage) are required to remove the protecting group. The thermally cleavable 5′-OH-protecting group preferably comprises an activator moiety and a cleavable linker moiety. The activator moiety is typically protected by a protecting group which is first removed under predetermined conditions, so as to expose a thermally cleavable deprotected activator and linker group, whereby on heating, the activator and linker group causes the protecting group to cleave, resulting in deprotection of the 5′—OH group.

In any aspect or embodiment of the present invention, the thermally cleavable 5′-OH-protecting group preferably comprises a safety catch protecting group, having one or two activator moieties and one or two cleavable linker moieties, wherein each activator moiety is protected with a protecting group, wherein the protecting group on each activator moiety is susceptible to deprotection under predetermined conditions, to expose the activator moiety, thereby rendering the activator moiety and cleavable linker moiety susceptible to cleavage on heating.

The starting nucleosides or nucleotides in step (i) are preferably attached to the surface of a solid substrate at the 3′-position via a thermally cleavable linker group. The thermally cleavable linker group may also be of the safety catch type. The thermally cleavable linker group may preferably comprise an activator moiety and a cleavable linker moiety, wherein the activator moiety can be protected by a protecting group which is removed under predetermined conditions to expose the activator moiety, thereby rendering the activator moiety and cleavable linker moiety susceptible to cleavage on heating.

In any aspect or embodiment of the present invention, the thermally cleavable linker group comprises one or two activator moieties and one or two cleavable linker moiety that on heating, causes the linker group to cleave, thereby causing detachment from the surface of the solid substrate.

More preferably, thermally cleavable linker group comprises a safety catch linker, having one or two activator moieties and one or two cleavable linker moieties, wherein the activator moiety is protected with a protecting group, wherein the protecting group on each activator moiety is susceptible to deprotection under predetermined conditions, to expose the activator moiety, thereby rendering the activator moiety and cleavable linker moiety susceptible to cleavage on heating.

The attachment of the starting nucleosides or nucleotides to the surface of the solid substrate is via the cleavable linker group.

Since the oligonucleotide synthesis of the present invention is carried out on a solid surface, the thermally cleavable linker group for attachment to the surface of the substrate preferably contains a protecting group that is removed under conditions which are orthogonal to all the conditions used in the oligonucleotide synthesis steps, since the linker group should stay intact during the entire synthesis. An advantage of a safety catch linker is that once the oligonucleotides have been prepared the protecting groups that protect the activator moiety can be removed at all the sites, resulting in a plurality of oligonucleotides that are bound to the surface of the substrate by thermally cleavable protecting groups. These oligonucleotides can be released under thermal means in a highly selective manner, thereby enabling a high degree of control over any subsequent oligonucleotide hybridisation process.

In steps (ii) and (iv), the 5′-OH protection either on the starting nucleoside or nucleotides or on the growing end of the oligonucleotide can be achieved by applying heat at the selected sites where the coupling reaction to grow the oligonucleotide is desired. With a thermally cleavable protecting group on the 5′-OH of the starting nucleoside, or nucleotide, or at the growing end of the oligonucleotide, each site can be selectively deprotected by the application of heat. The highly selective application of heat to the selected reaction sites enables the coupling reactions to be performed with high fidelity. Preferably there is substantially no deprotection of the 5′-OH protecting groups at sites other than the selected sites. By “substantially no deprotection”, it is meant that: <0.5%, <0.4%, <0.3%, <0.2%, <0.1%, or none, of the 5′-OH protected groups at sites other than the selected sites are deprotected.

In the coupling steps (iii) and (v), the deprotected 5′—OH groups of the starting nucleoside, or oligonucleotide, or on the growing end of the oligonucleotide, respectively, at the selected sites are subjected to a coupling reaction with a nucleoside/nucleoside building block or a nucleotide building block comprising a 5′-OH protecting group. Since the deprotection is selective for the selected sites, the possibility of unintended coupling or side reactions at the sites other than the selected sites is greatly reduced, or even eliminated. Preferably, the coupling steps (iii) and (v) comprise contacting a solution containing the incoming nucleoside/nucleoside or nucleotide building block comprising a 5′-OH protecting group, with the surface of the substrate, wherein the nucleoside, nucleoside building block or nucleotide building block reacts with the deprotected 5′—OH groups at the selected sites. The sites other than the selected sites are not heated, or may be subjected to cooling, in order to further minimize the possibility of unintended reactions at those sites. Preferably there is substantially no reaction with the incoming nucleoside/nucleoside building block or nucleotide building block at sites other than the selected sites. By “substantially no reaction”, it is meant that: <0.5%, <0.4%, <0.3%, <0.2%, <0.1%, or none, of the sites other than the selected sites react with the incoming nucleoside, nucleoside building block, or nucleotide building block.

### Attachment of the First Nucleoside

In step (i), each site on the surface of the solid substrate is provided with a plurality of nucleosides or nucleotides (preferably nucleosides) comprising a 5′-OH protecting group, wherein the nucleosides or nucleotides are immobilized on the surface of a solid substrate. As indicated above, the surface of the solid substrate is provided with nucleosides (“starting nucleosides”) or nucleotides (“starting nucleotides”—optionally di- or tri-nucleotides) which are bound to the surface to form a “reaction site”. Within a single reaction site there may be a plurality of the same starting nucleoside or nucleotide each bound to the surface of the substrate. Different reaction sites may comprise different surface-bound nucleosides or nucleotides, depending on the desired oligonucleotides to be synthesised.

Preferably, the 5′-OH-protected nucleosides or nucleotides of step (i) comprise a thermally cleavable 5′-OH-protecting group, and the nucleosides or nucleotides are attached to the surface of a solid substrate at the nucleoside 3′-position (or nucleotide 3′-position) via a thermally cleavable linker group, wherein the thermally cleavable linker attaching the first nucleoside to the surface is stable to removal during the oligonucleotide synthesis steps.

Step (i) preferably comprises providing at each site, a plurality of nucleosides immobilized to the solid surface, each immobilized nucleoside being represented by:

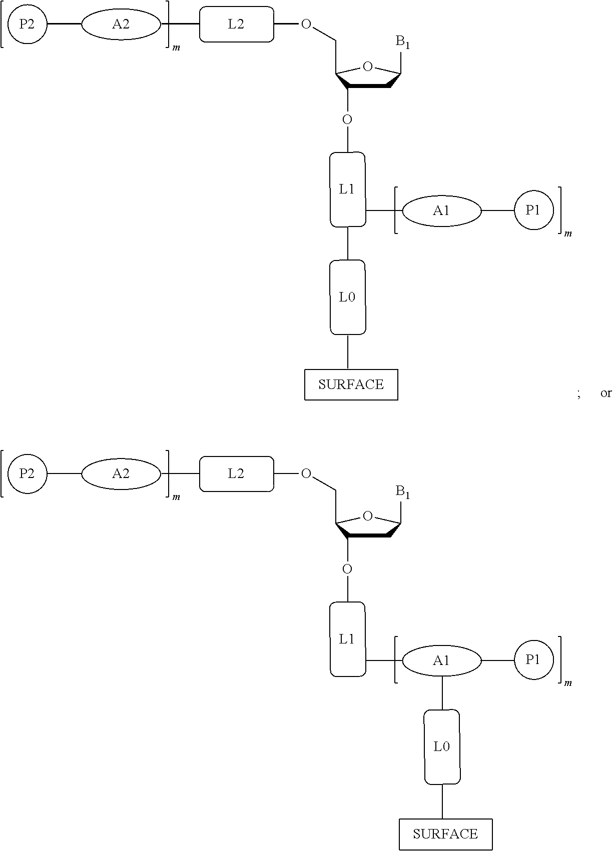

wherein:
L1-A1-P1 together represents a safety catch linker for attachment at the 3′—OH group of the nucleoside to the surface, wherein:
   P1 represents a protecting group,
   L1 represents a thermally cleavable linker moiety,
   A1 represents an activator moiety that, upon removal of P1, is capable of causing cleavage of the cleavable linker from the solid surface;
P2-A2-L2 together represents a safety catch 5-OH-protecting group, wherein:
   P2 represents a protecting group,
   L2 represents a cleavable linker moiety,
   A2 represents an activator moiety that, upon removal of P2, is capable of causing removal of the 5′-OH protecting group;
m at each occurrence is the same or different, and represents 1 or 2;
L0 represents a moiety for attachment of the first nucleoside via the cleavable linker group to the surface; and
B₁ represents an optionally protected canonical or an optionally protected non-canonical nucleobase,
   wherein A1, A2, L1 and L2 can be the same or different, and wherein P1 and P2 are different and are removable under different conditions or reagents. Preferably, the protecting groups on the nucleobases, when present, are stable to removal during the oligonucleotide synthesis. Similarly, the protecting groups P4 on the nucleoside 3′-phosphoramidites are stable to removal during the oligonucleotide synthesis. The nucleobase protecting groups may preferably be removed at the end of the oligonucleotide synthesis, along with the phosphate protecting group (e.g. P4).

In any embodiment or aspect of the present invention, the attachment or immobilization of the starting nucleosides or nucleotides to the solid surface via the L0 moiety may be at any suitable part of the safety catch linker L1-A1-P1, that enables the oligonucleotide to be cleaved from the surface of the substrate at the end of the oligonucleotide synthesis, e.g. at L1 or A1. The attachment to the substrate via the L0 moiety is preferably via on any suitable atom in the L1 or A1 moiety. For example, where m represents 2, it will be appreciated that the attachment or immobilisation of the starting nucleosides or nucleotides to the solid surface via the L0 moiety to A1 as depicted above, is at a single point/atom on one of the A1 groups and not at both A1 groups. Thus, at the end of the oligonucleotide synthesis, the safety catch linker enables complete detachment of the oligonucleotide from the surface of the substrate, i.e. to release an oligonucleotide which preferably contains a 3′-hydroxyl group. Preferably, the safety catch linker is also completely detached from the substrate.

There may be a plurality of the same immobilized nucleosides or nucleotides (preferably di- or tri-nucleotides) at each reaction site of the solid surface. At adjacent reaction sites of the solid surface, the immobilized nucleosides or nucleotides may be the same or different.

The preparation of the plurality of nucleosides immobilized to the solid surface, according to step (i) preferably comprises staged attachment of each different nucleoside to the surface. In particular, step (i) preferably comprises:
(a) providing a solid surface comprising a plurality of sites, wherein each site is functionalized with thermally labile linker groups, each of which is represented by:

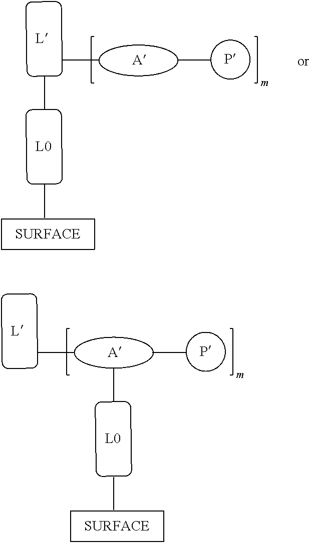

wherein:
L′-A′-P′ together represents a safety catch linker attached to the surface via L0, wherein:
   P′ represents a protecting group for the activator moiety,
   L′ represents a cleavable linker moiety,
   A′ represents an activator moiety that, upon removal of P′, is capable of causing cleavage of the cleavable linker group from the solid surface;
m=1 or 2;
L0 represents a moiety for attachment of the cleavable linker group to the surface;
(b) removing the protecting group P′, thereby resulting in a solid surface comprising a plurality of sites represented by:

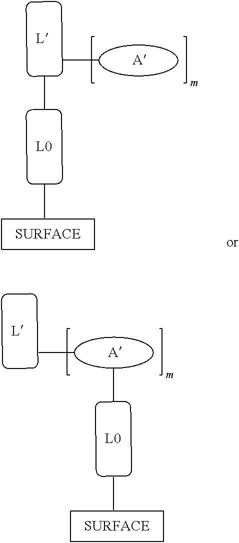

(c) thermally controlled deprotection of the cleavable linker L′ via activator moiety A′ at selected sites on the solid surface, and coupling the deprotected sites with a nucleoside (“starting nucleoside”) represented by the formula:

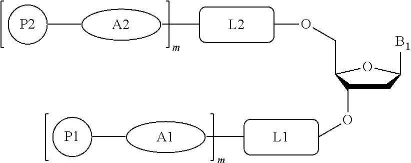

wherein:
   L1-A1-P1 together represents a safety catch linker for attachment at the 3-OH group of the first nucleoside to the surface, wherein:
      P1 represents a protecting group,
      L1 represents a cleavable linker moiety,
      A1 represents an activator moiety that, upon removal of P1, is capable of causing cleavage of the cleavable linker from the solid surface;
   P2-A2-L2 together represents a safety catch 5′-OH protecting group, wherein:
      P2 represents a protecting group,
      L2 represents a cleavable linker moiety,
      A2 represents an activator moiety that, upon removal of P2, is capable of causing removal of the 5-OH protecting group; and
m at each occurrence is the same or different, and represents 1 or 2; and
B₁ represents an optionally protected canonical or an optionally protected non-canonical nucleobase [preferably wherein the nucleobase is one of: adenine (A), cytosine (C), guanine (G) or thymine (T)],
(d) thermally controlled deprotection of the cleavable linker L′ via activator moiety A′ at selected sites which were not deprotected in the preceding step and coupling the deprotected sites with another nucleoside, preferably a nucleoside comprising one of the other three canonical nucleobases; and
(e) repeating step (d) with the other remaining nucleosides;
thereby forming a plurality of sites on a solid surface wherein the solid surface comprises a plurality of 5′-OH-protected nucleosides (protected with a safety catch protecting group -L2-A2-P2) containing nucleobases, wherein the nucleobases are optionally protected canonical or optionally protected non-canonical nucleobases [preferably wherein the nucleobases are A, C, G and T] and wherein the nucleosides are each attached at the 3′-OH to the solid surface via a cleavable linker group -L1-A1-P1-.

The preparation of the plurality of nucleosides immobilized to the solid surface, according to step (i) preferably comprises:
(a) providing a solid surface comprising a plurality of sites, wherein each site is functionalized with thermally labile linker groups, each of which is represented by:

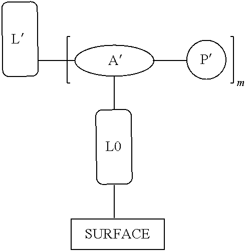

wherein:
   L′-A′-P′ together represents a safety catch linker attached to the surface via L0, wherein:
      P′ represents a protecting group for the activator moiety,
      L′ represents a cleavable linker moiety,
      A′ represents an activator moiety that, upon removal of P′, is capable of causing cleavage of the cleavable linker group from the solid surface;
   m=1 or 2;
   L0 represents a moiety for attachment of the cleavable linker group to the surface;
(b) removing the protecting group P′, thereby resulting in a solid surface comprising a plurality of sites represented by:

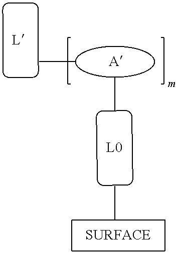

(c) thermally controlled deprotection of the cleavable linker L′ via activator moiety A′ at selected sites on the solid surface, and coupling the deprotected sites with a nucleoside (“starting nucleoside”) represented by the formula:

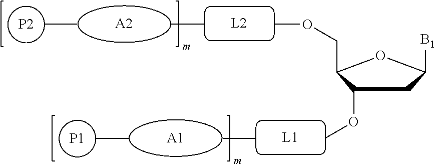

wherein:
   L1-A1-P1 together represents a safety catch linker for attachment at the 3-OH group of the first nucleoside to the surface, wherein:
      P1 represents a protecting group,
      L1 represents a cleavable linker moiety,
      A1 represents an activator moiety that, upon removal of P1, is capable of causing cleavage of the cleavable linker from the solid surface;
   P2-A2-L2 together represents a safety catch 5′-OH protecting group, wherein:
      P2 represents a protecting group,
      L2 represents a cleavable linker moiety,
      A2 represents an activator moiety that, upon removal of P2, is capable of causing removal of the 5-OH protecting group; and
m at each occurrence is the same or different, and represents 1 or 2; and
B₁ represents an optionally protected canonical or an optionally protected non-canonical nucleobase [preferably wherein the nucleobase is one of: adenine (A), cytosine (C), guanine (G) or thymine (T)],
(d) thermally controlled deprotection of the cleavable linker L′ via activator moiety A′ at selected sites which were not deprotected in the preceding step and coupling the deprotected sites with another nucleoside, preferably a nucleoside comprising one of the other three canonical nucleobases; and
(e) repeating step (d) with the other remaining nucleosides;
   thereby forming a plurality of sites on a solid surface wherein the solid surface comprises a plurality of 5-OH-protected nucleosides (protected with a safety catch protecting group -L2-A2-P2) containing nucleobases, wherein the nucleobases are optionally protected canonical or optionally protected non-canonical nucleobases [preferably wherein the nucleobases are A, C, G and T] and wherein the nucleosides are each attached at the 3′-OH to the solid surface via a cleavable linker group -L1-A1-P1-.

Alternatively, in any of the above embodiments, step (c) may comprise thermally controlled deprotection of the cleavable linker L′ via activator moiety A′ at selected sites on the solid surface, and coupling the deprotected sites with a nucleotide (“starting nucleotide”) which may be a di-nucleotide represented by the formula:

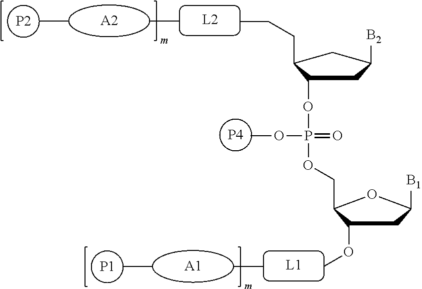

wherein:
   L1-A1-P1 together represents a safety catch linker for attachment at the 3′-OH group of the first nucleoside to the surface, wherein:
      P1 represents a protecting group,
      L1 represents a cleavable linker moiety,
      A1 represents an activator moiety that, upon removal of P1, is capable of causing cleavage of the cleavable linker from the solid surface;
   P2-A2-L2 together represents a safety catch 5′-OH protecting group, wherein:
      P2 represents a protecting group,
      L2 represents a cleavable linker moiety,
      A2 represents an activator moiety that, upon removal of P2, is capable of causing removal of the 5′-OH protecting group;
   P4 represents a phosphate protecting group;
   m at each occurrence is the same or different, and represents 1 or 2; and
B₁ and B₂ may be the same or different, and each independently represents an optionally protected canonical or an optionally protected non-canonical nucleobase, preferably wherein the nucleobase is one of: adenine (A), cytosine (C), guanine (G) or thymine (T).

Alternatively, in any of the above embodiments, step (c) may comprise thermally controlled deprotection of the cleavable linker L′ via activator moiety A′ at selected sites on the solid surface, and coupling the deprotected sites with a nucleotide (“starting nucleotide”) which may be a tri-nucleotide represented by the formula:

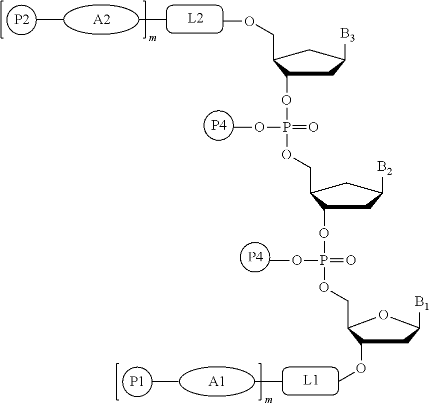

wherein:
   L1-A1-P1 together represents a safety catch linker for attachment at the 3′-OH group of the first nucleoside to the surface, wherein:
      P1 represents a protecting group,
      L1 represents a cleavable linker moiety,
      A1 represents an activator moiety that, upon removal of P1, is capable of causing cleavage of the cleavable linker from the solid surface;
   P2-A2-L2 together represents a safety catch 5′-OH protecting group, wherein:
      P2 represents a protecting group,
      L2 represents a cleavable linker moiety,
      A2 represents an activator moiety that, upon removal of P2, is capable of causing removal of the 5′-OH protecting group;
   each P4 can be the same or different, and each independently represents a phosphate protecting group;
   m at each occurrence is the same or different, and represents 1 or 2; and
   B₁, B₂ and B₃ may be the same or different, and each independently represents an optionally protected canonical or an optionally protected non-canonical nucleobase, preferably wherein the nucleobase is one of: adenine (A), cytosine (C), guanine (G) or thymine (T).

In any of the above-described aspects and embodiments, in step (b) the protecting group P′ is preferably removed from all sites of the surface. The resulting surface will comprise a plurality of thermally cleavable protecting groups (L‘-A’) which can be cleaved under thermal control at the sites where a reaction is desired (initially at the sites where the first nucleoside or nucleotide is to be coupled). The deprotected 5′—OH groups at the selected sites are then coupled with a 5′-OH protected nucleoside or 5′-OH protected nucleotides [step (c)], so as to prepare surface-attached 5′-OH protected nucleosides or 5′-OH protected nucleotides. In the subsequent steps, other selected sites on the surface are subjected to thermal deprotection of the 5′—OH group, and the resulting deprotected groups are reacted with a different 5′-OH protected nucleoside or 5′-OH protected nucleotide [step (d)]. The deprotection/coupling steps are repeated until all the desired sites on the surface of the substrate are populated with the desired 5′-OH protected nucleoside or 5′-OH protected nucleotide forming the starting nucleoside/nucleotide for independent, parallel synthesis of the oligonucleotides.

In a preferred embodiment of the oligonucleotide synthesis of the present invention, step (i) comprises preparing a plurality of nucleosides immobilized to the solid surface.

### Surface Attachment

In order to provide the thermal control at the various stages of the oligonucleotide synthesis process (attachment of the first nucleoside or nucleotide to the surface of the substrate, deprotection of the first and subsequent 5′-OH growing ends of the nucleosides/oligonucleotides and/or release of the synthesised oligonucleotides) the surface of the substrate is preferably coated in an electrically conductive material, such as gold or silicon. The substrate may comprise a gold or silicon surface with individually thermally addressable sites on a chip. A substrate comprising a silicon surface is particularly preferred.

Methods of attaching a functionalised moiety to a gold or silicon surface are known. For example, attachment to a gold or silicon surface can be achieved via an association with a functionalised carbene or a functionalised alkyne, preferably a functionalised alkyne. Preferably, the attachment is to a silicon surface via a functionalised alkyne. Examples of suitable surface attachments are described in detail below:

### (A) Attachment to Silicon Surface

### (A1) Attachment to Hydrogen-Passivated Silicon

One approach uses the formation of self-assembled monolayers on hydrogen-passivated, oxide free silicon (silicon oxide is an efficient thermal insulator, and so detrimental to thermal control between reaction sites). Alkyl or alkenyl monolayers are formed by the grafting of 1-alkenyl or preferably 1-alkynyl species under thermal, photochemical [e.g. as described in U.S. Pat. No. 6,465,054 B2], or preferably by electrografting conditions [e.g. as described in Buriak Chem. Rev. 2002, 102, 1271, U.S. Pat. No. 6,485,986 B1, U.S. Pat. No. 7,521,262 B2, U.S. Pat. No. 6,846,681 B2], following the removal of the native silicon oxide by exposure to 3% aq.HF or 40% aq.NH₄F, in accordance with the following Scheme 1:

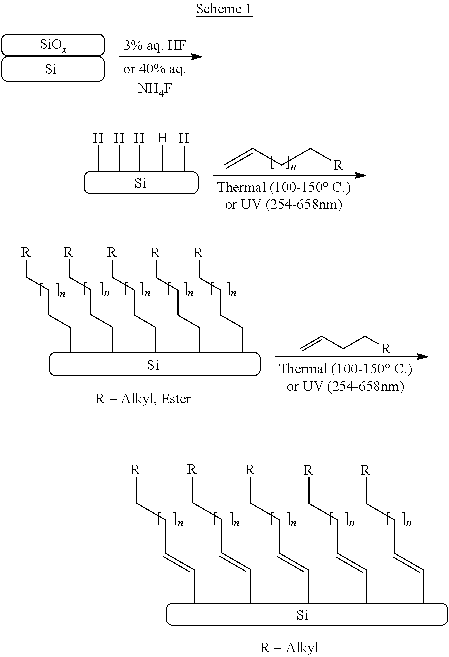

Thermal and photochemical initiations proceed via a free radical mechanism, and are performed under anoxic conditions using neat degassed 1-alkyne or 1-alkene (photochemical and thermal routes), or using solutions in high boiling aromatic solvents such as toluene or mesitylene (thermal route).

Photochemical monolayer formation from 1-alkyne and 1-alkenes using 447 nm irradiation [J.Am.Chem.Soc. 2005, 127, 2514] was found to give monolayers of comparable quality, as assessed by water contact angle, X-ray reflectivity and XPS, to those obtained by thermal initiation. There was no observable Si—O formation indicative of oxidation of unreacted Si—H surface species. Comparison of photochemical formation of 1-alkene and 1-alkyne monolayers under 371-658 nm irradiation to those using 254 nm light gave comparable monolayer quality, but with no observable photochemical side-products.

### (A2) Formation of DNA Surfaces on H-Passivated Silicon by Immobilisation of Pre-Synthesised Oligonucleotides

Photochemical formation of 1-alkene and 1-alkyne monolayers containing chemically derivatisable groups has been shown to provide suitable platforms for the attachment of pre-synthesised oligonucleotides. U.S. Pat. No. 6,677,163 B1 describes the formation of monolayers containing protected chemically derivitisable groups (OH, NH₂, CO₂H) by the thermal or photochemical reaction of 1-alkenes with H—Si surfaces according to following monolayer formation, the chemically-derivitisable terminal group is either deprotected or activated (e.g. by formation of a succinimidyl ester) and subsequently reacted with a biomolecule (such as ssDNA) bearing a reactive coupling group to form oligonucleotide and other biomolecule bearing surfaces (Scheme 2).

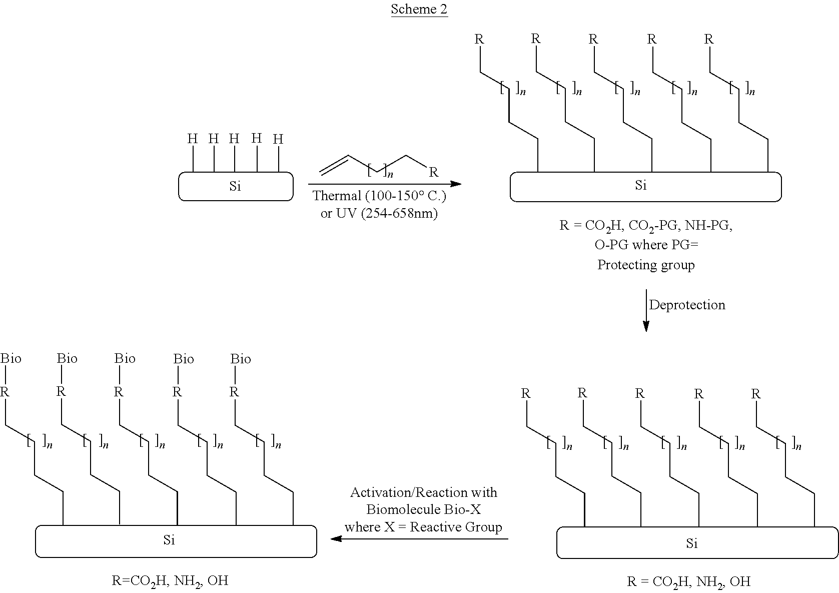

This approach was employed to install activated succinimidyl ester monolayers for reaction with amino-functionalised oligonucleotides [Nucl. Acids. Res. 2004, 32, e118] in submicron patterns on Si (100), as well as formation of the same activated ester by installation and deprotection of a methyl-ester terminated film [Microelectronic Eng. 2004, 73-74, 830] using 248 nm photochemical functionalisation. Similarly, electrografting of 1-alkynes containing terminal carboxylate functionalities [Nucl Acid Res. 2006, 34, e32] has been used as a platform to immobilise reactive oligonucleotides, via the formation of intermediate succimidyl active esters. Use of terminal carboxylates can also provide a binding functionality for intermediate poly (lysine) films, which were then activated using a maleimide-containing crosslinker SSMCC [J.Am.Chem.Soc. 2000, 122, 1205]. The resulting maleimide functionalised surface is able to capture thiolated ssDNA for fluorescence studies. Direct installation of non-biofouling oligo(ethylene glycol) (OEG) monolayers with reactive terminal epoxide groups by thermal initiated film formation was used to produce highly thermally stable films. The reaction of the epoxide termini with thiolated ss-DNA allowed the formation of DNA films on Si, which were probed by hybridisation with complementary fluorescently labelled 3′-TAMRA ssDNA [Langmuir 2006, 22, 3494].

Similarly, non-fouling OEG-terminated alkyl monolayers formed by photochemical grafting of OEG-terminated 1-alkenes to H-passivated silicon were used as a platform for biomolecule (including oligonucleotide) immobilisation [U.S. Pat. No. 9,302,242 B2]. In this approach, discrete spatially resolved nanowells were created by anodic lithography using a conducting AFM probe by chain scission of the OEG moiety. These regions were then susceptible to further derivitisation, allowing the attachment of oligonucleotides, proteins, and avidin.

In a further example, biosensors based on the modification of field-effect transistor (FET) gate electrodes by grafting of 1-alkene and 1-alkyne monolayers containing reactive termini have been demonstrated [U.S. Pat. No. 7,507,675 B2]. This approach is not limited to crystalline silicon substrates; in a further example [US 2012/0142045] the grafting of chemically functionalizable 1-alkenes to oxide-free amorphous silicon (a-Si) and silicon carbide (SiC) films coated on Au plasmonic nanostructures. Further derivatisation of the grafted chain termini to introduce biomolecule ligands, such as oligonucleotides as an example, are demonstrated.

### (A3) Direct Oligonucleotide Synthesis on Hydrogen-Passivated Silicon

Oligonucleotide synthesis has previously been performed using terminal dimethoxytrityl (DMT) protected alkylhydroxyl monolayers on oxide-free Si surfaces [ACIE. 2002, 41, 615](Scheme 3):

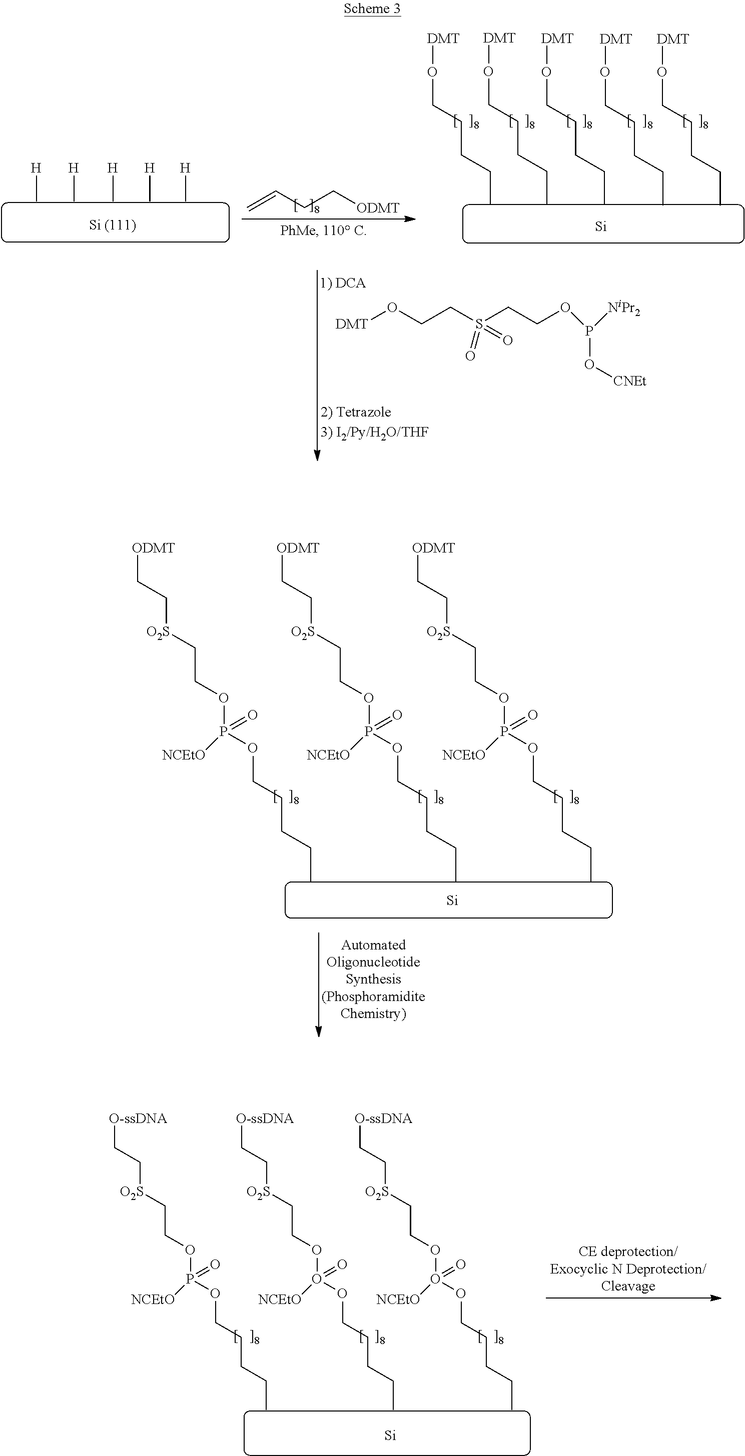

Terminal DMT-O monolayers were formed from the corresponding 11-DMToxy-1-alkene under thermal initiation. The resulting DMT-O terminated monolayer films were then exposed to automated phosphoramidite synthesis to first install a base-cleavable linker and then to synthesise a 17-mer oligonucleotide (5′-CGGCATCGTACGATTAT (SEQ ID NO: 1)), which was cleaved during deprotection. The ssDNA surface density was measured as 3.19×10¹² strands/cm² by Ru[(NH₃)]³⁺ binding studies. Surface hybridisation of a complementary 18-mer, followed by intercalation of methylene blue allowed determination of the dsDNA surface density. The dsDNA surface density of 1.05×10¹² strands/cm² showed that 33% of the surface bound ssDNA had undergone hybridisation. This strategy was also used to install a C5-ethynylferrocene-dC at the 3′-terminus [Chem.Eur.J. 2005, 11, 344; J.Electroanalytical Chem. 2007, 603, 67] which enabled charge transfer studies of the surface-bound oligonucleotide. Direct imaging of surface-bound ss- and ds-DNA on H—Si was also performed using oligonucleotides synthesised using this methodology [Langmuir 2003, 19, 5457].

### (A4) Synthesis of Candidate Compounds for Attachment to Hydrogen-Passivated Silicon Substrates

The proposed strategy involves the formation of a monomolecular film containing a safety catch thermally-cleavable protecting group at the monolayer termini (Scheme 4):

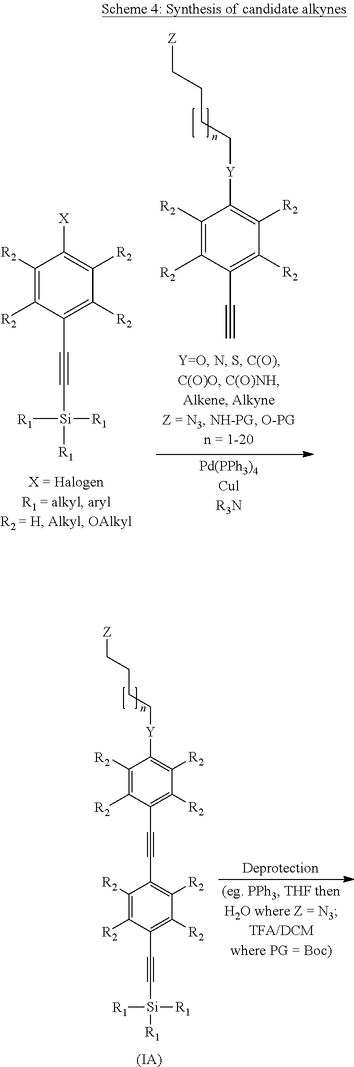

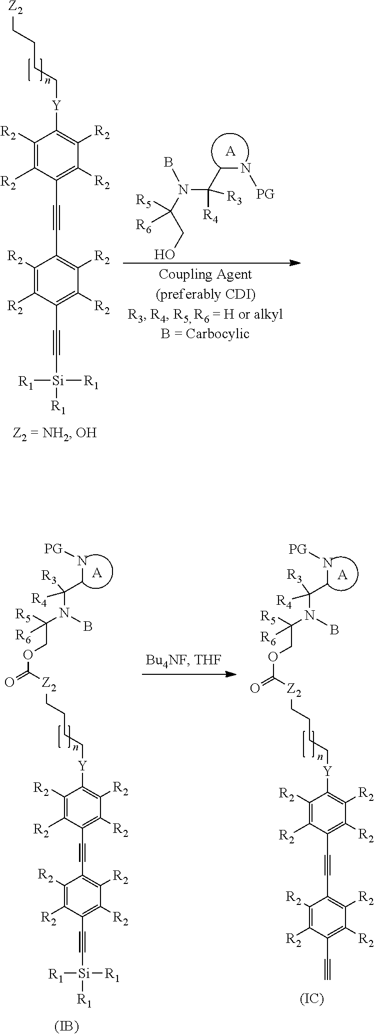

The candidate alkynes can be deposited onto hydrogen passivated silicon via the following Scheme 5:

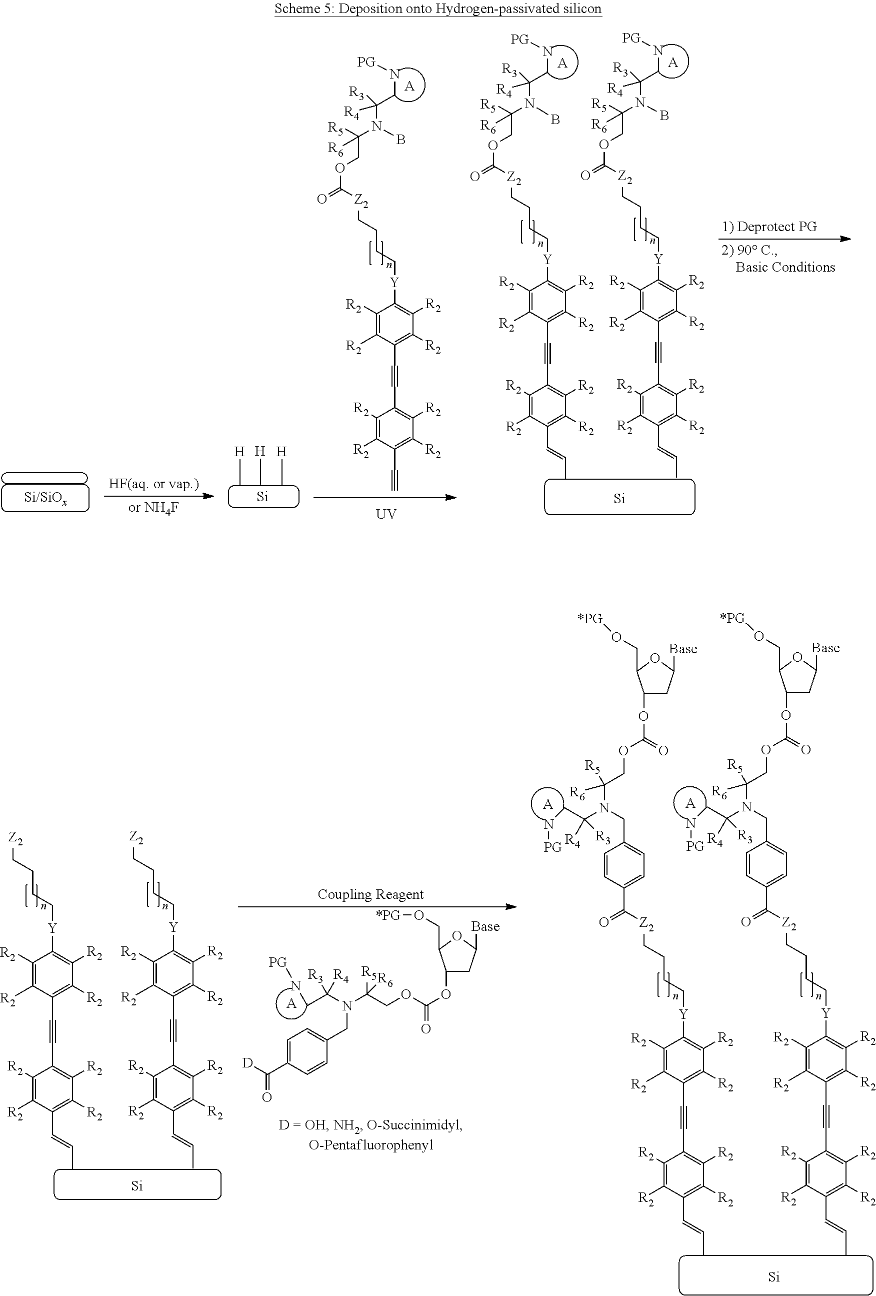

Removal of the safety catch and deprotection under basic conditions to reveal a chemically-derivitisable group Z2 then allows the installation of a safety-catch thermally cleavable linker-first base conjugate through appropriate coupling chemistry, such as activated ester formation or peptide coupling.

In a further strategy (Scheme 6), derivatisation of compound (IA), where Z=N₃ allows installation of the safety catch thermally-cleavable linker-first base conjugate via Click Chemistry [for example J.Am.Chem.Soc. 2005, 127, 210; Langmuir 2006, 22, 24571:

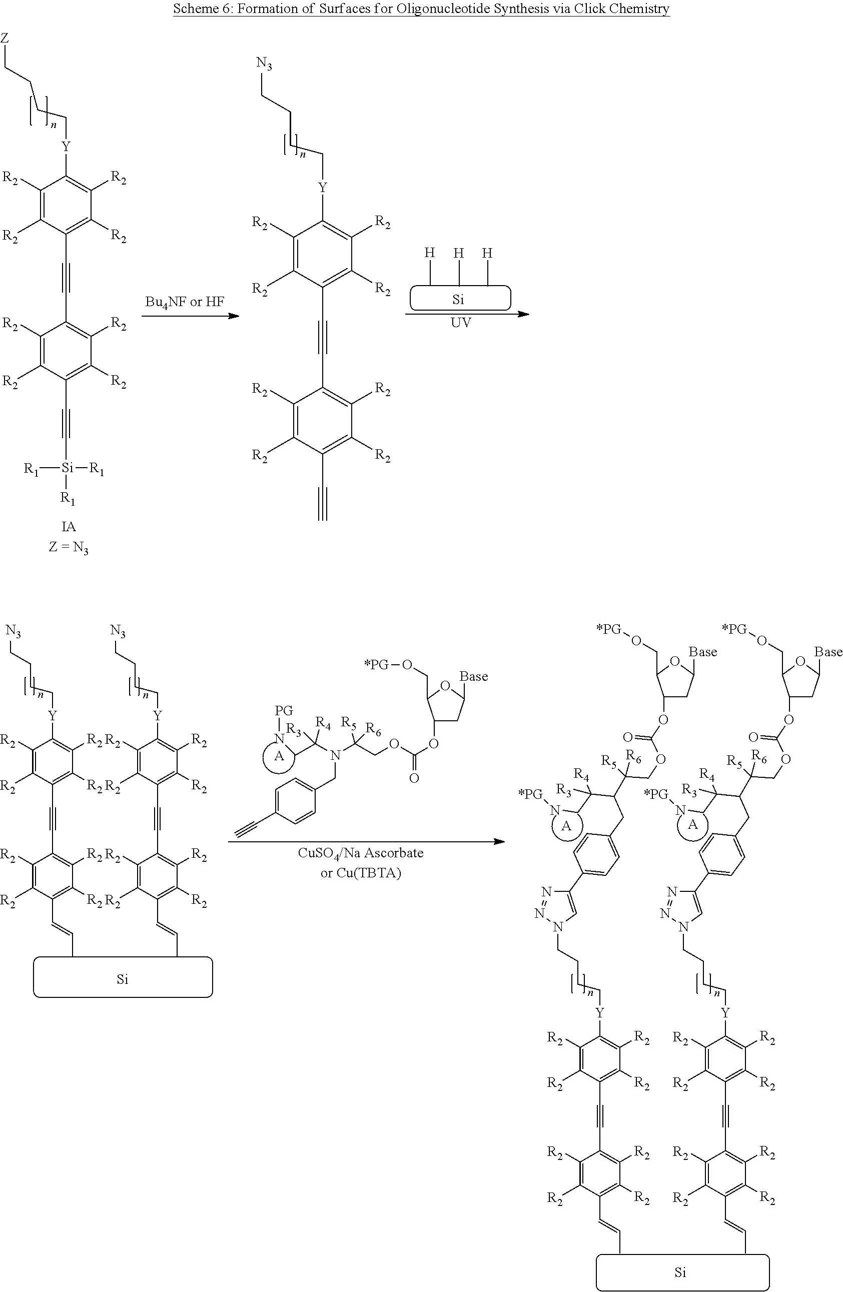

### (B)—Attachment to Gold Surface

### (B) Carbene and Thiols on Gold Substrate

Metal such as gold have been attractive as a substrate to grow self-assembled monolayers (SAMs) because of their thermal conductivity and have been described for their wide potential and especially in the field of biosensors. In particular, carbenes on gold [Crudden, Nat. Chem., vol. 6, 409-414, 2014] have been described for their enhanced stability compared to their thiols counterparts [C. Vericat, et al. Chem Soc. Rev. 39, 1805 (2010); Johnson, WO2014/160471A2].

### (B1) Candidate Structures for Carbene Attachment to Gold

Proposed reaction schemes are found below for the synthesis of suitable candidate compounds for attachment to gold substrates. The detailed strategy involves the installation of a SAM containing a safety catch thermally-cleavable protecting group at the monolayer termini. Removal of the safety catch and deprotection under basic conditions reveals a chemically derivatisable group Z2 (Scheme 7).

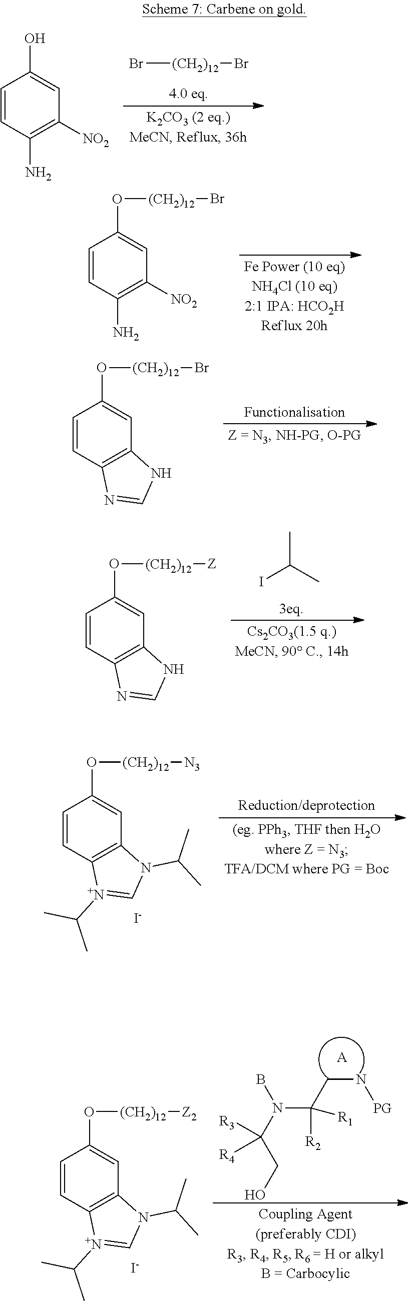

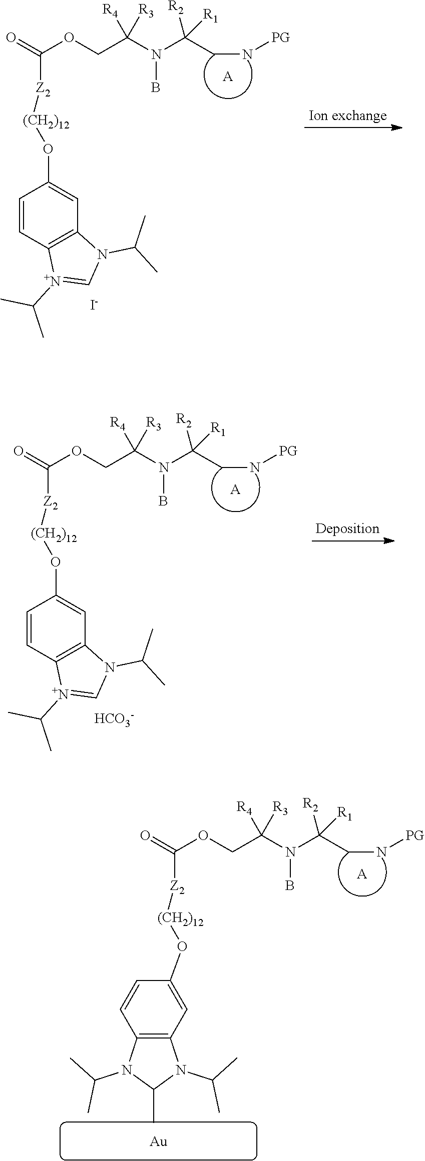

The first nucleoside may then be attached according in a manner as exemplified by the following Scheme 8:

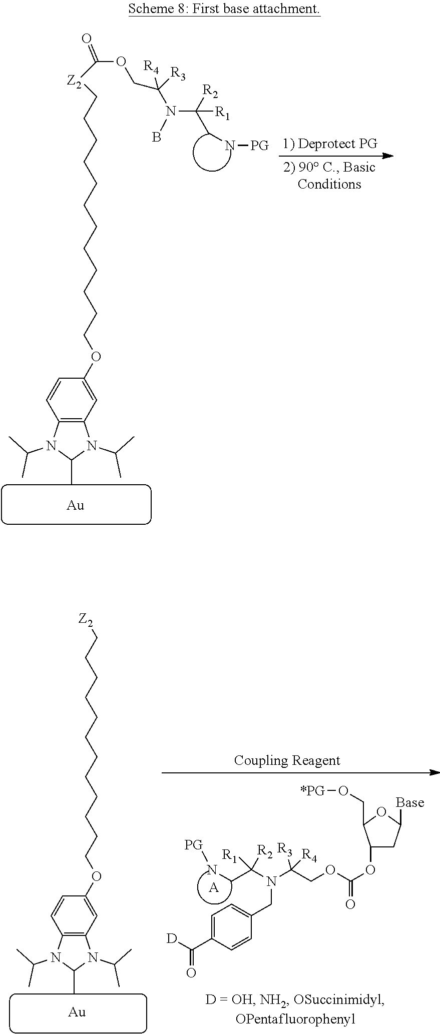

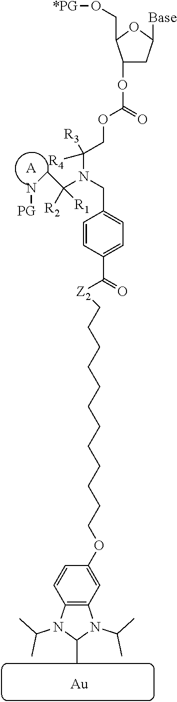

### (B2) Candidate Structures for Thiol Attachment to Gold

The attachment of a functionalised thiol to a gold surface may be achieved via the following Scheme 9:

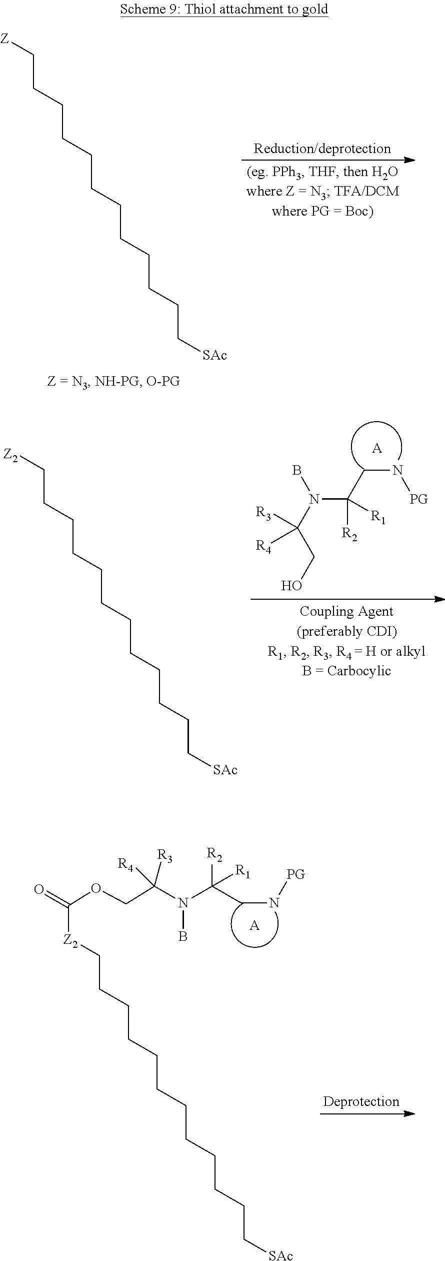

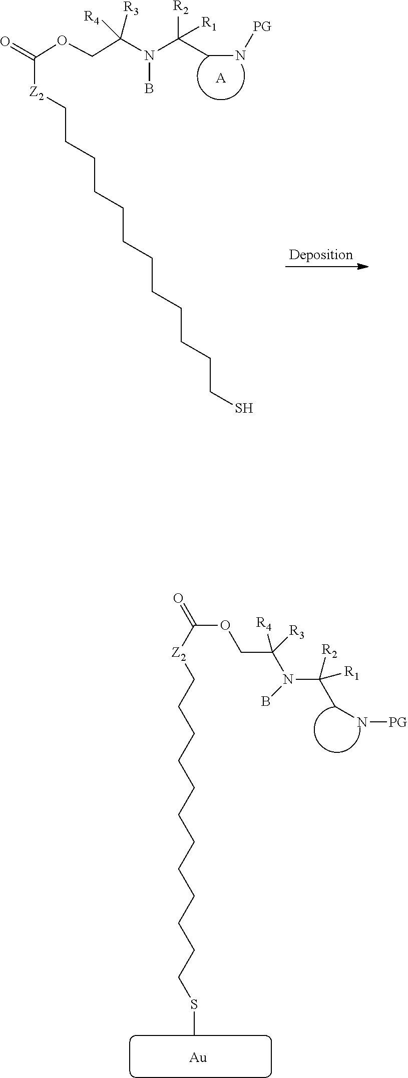

The first nucleoside may then be attached according in a manner as exemplified by the following Scheme 10:

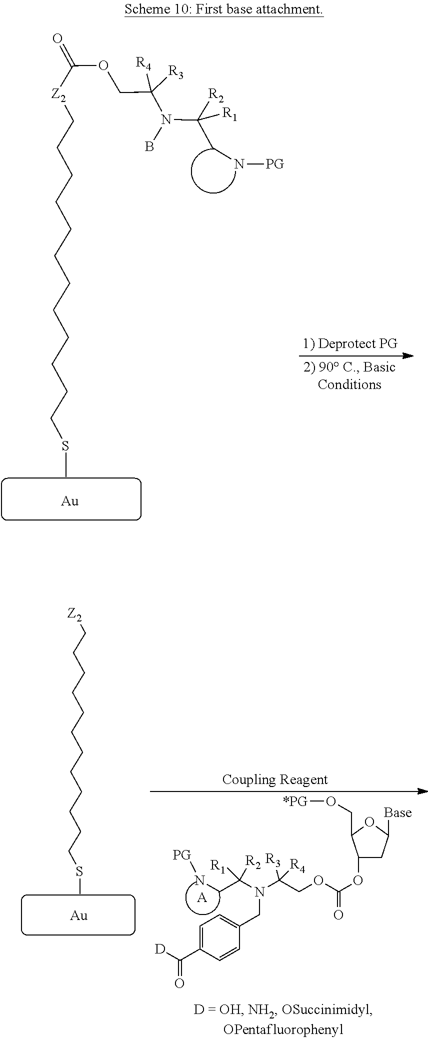

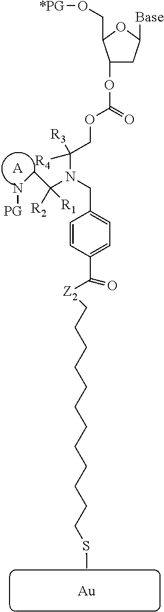

### Oligonucleotide Synthesis Process

As explained above, in order to enable thermally controlled addition of first nucleosides or nucleotides to the surface of the reaction sites, each reaction site is coated with a surface that allows attachment of the growing oligonucleotide fragments, but is chemically inert to all of the processes used for the DNA synthetic process.

Non-thermally controlled functionalisation of the surfaces of all the reaction sites is then carried out. This functionalisation attaches a group containing a reactive moiety that has been protected with a thermally labile protecting group to the surfaces.

Following deprotection of the thermally labile protecting group, the surface-attached reactive moiety can then react with an incoming molecule comprising a complimentary reactive moiety attached to a first nucleoside or nucleotide via a thermally cleavable linker attached at the 3′-position, where the 5′-hydroxy group is protected with a thermally cleavable protecting group.

The conditions used for deprotecting the surface-attached reactive moiety are orthogonal to the conditions used for cleaving the thermally cleavable linker and deprotecting the 5′-hydroxyl protecting group, but only at the cold temperature. This process can be repeated until every required first nucleoside or nucleotide functionalises the reaction sites. For example, following functionalisation of reaction sites with a nucleoside or nucleotide, the process may be repeated until all the desired reaction sites are populated with the required nucleosides or nucleotides. On completion of this phase the oligonucleotide fragment synthesis can commence. Preferably, the reaction sites are functionalized with nucleosides.

Thermally controlled synthesis of oligonucleotide fragments from the surface-attached and protected first nucleoside or nucleotide is achieved by first deprotecting the thermally labile protecting group on the 5′-hydroxy of the first nucleoside or nucleotide. Thus, in the oligonucleotide synthesis process of the present invention, step (ii) comprises thermally controlled removal of the safety catch 5′-OH protecting cleavable linker group P2-A2-L2.

Reaction sites that have been heated will then contain nucleosides or nucleotides with a deprotected 5′-hydroxy group, which can then react with an incoming nucleoside or nucleotide that is protected on the 5′-hydroxy group with a thermally sensitive protecting group. This process can be repeated until every required oligonucleotide is produced.

Preferably, the incoming nucleoside or nucleotide is a nucleoside or nucleotide comprising a 5′-OH protecting group in step (iii) and step (v) is a nucleoside 3′-phosphoramidite, a di-nucleotide 3′-phosphoramidite or a tri-nucleotide 3′-phosphoramidite comprising a thermally cleavable 5′-OH protecting group. Preferably, the thermally cleavable 5′-OH-protecting group comprises one or two activator moieties and one or two cleavable linker moieties, that on heating, causes the protecting group to cleave thereby resulting in deprotection of the 5′—OH group. More preferably, the thermally cleavable 5′-OH-protecting group comprises a safety catch linker, having one or two activator moieties and one or two cleavable linker moieties, wherein each activator moiety is protected with a protecting group, wherein the protecting group on each activator moiety is susceptible to deprotection under predetermined conditions, to expose the activator moiety, thereby rendering the activator moiety and cleavable linker moiety susceptible to cleavage on heating.

Preferably, in any aspect or embodiment of the present invention, the nucleoside comprising a 5′-OH-protecting group in step (iii) and step (v) is a nucleoside 3′-phosphoramidite comprising a thermally cleavable 5′-OH protecting group represented by:

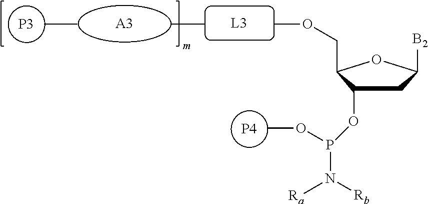

wherein:
   P3-A3-L3 together represents a safety catch 5-OH-protecting group, wherein:
      P3 represents a protecting group
      L3 represents a cleavable linker moiety,
      A3 represents an activator moiety that, upon removal of P3, is capable of causing removal of the 5′-OH protecting group;
   m=1 or 2;
   P4 represents a phosphoramidite protecting group;
   B₂ represents an optionally protected canonical or an optionally protected non-canonical nucleobase; and
   Rₐ and R_{b} can be the same or different and each represent alkyl.

In any aspect or embodiment of the present invention, the nucleotide comprising a 5′-OH-protecting group in step (iii) and step (v) may be a di-nucleotide 3′-phosphoramidite comprising a thermally cleavable 5′-OH protecting group represented by:

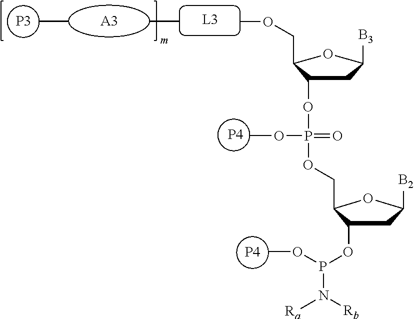

wherein:
   P3-A3-L3 together represents a safety catch 5′-OH-protecting group, wherein:
   P3 represents a protecting group
   L3 represents a cleavable linker moiety,
   A3 represents an activator moiety that, upon removal of P3, is capable of causing removal of the 5′-OH protecting group;
      m=1 or 2;
      each P4 may be the same or different and represents a phosphoramidite or phosphate protecting group;
      B₂ and B₃ may be the same or different and each independently represents an optionally protected canonical or an optionally protected non-canonical nucleobase; and
      Rₐ and R_{b} may be the same or different and each represents alkyl.

In any aspect or embodiment of the present invention, the nucleotide comprising a 5′-OH-protecting group in step (iii) and step (v) is a tri-nucleotide 3′-phosphoramidite comprising a thermally cleavable 5′-OH protecting group represented by:

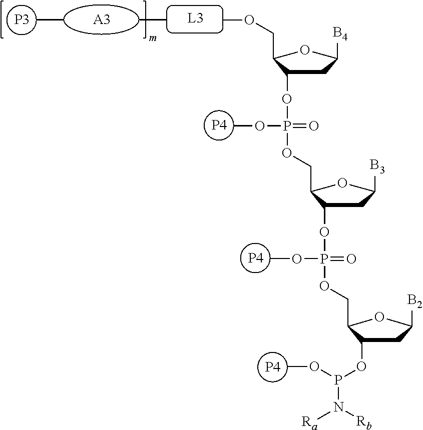

wherein:
   P3-A3-L3 together represents a safety catch 5′-OH-protecting group, wherein:
      P3 represents a protecting group
      L3 represents a cleavable linker moiety,
      A3 represents an activator moiety that, upon removal of P3, is capable of causing removal of the 5′-OH protecting group;
   m=1 or 2;
   each P4 may be the same or different and each represents a phosphoramidite or phosphate protecting group;
   B₂, B₃ and B₄ may be the same or different and each independently represents an optionally protected canonical or an optionally protected non-canonical nucleobase; and
   Rₐ and R_{b} may be the same or different and each represents alkyl.

Preferably, the coupling of the nucleoside 3′-phosphoramidite comprising a 5′-OH protecting group in step (iii) to a deprotected 5′—OH group of the immobilized nucleoside, followed by oxidation, forms a structure represented by:

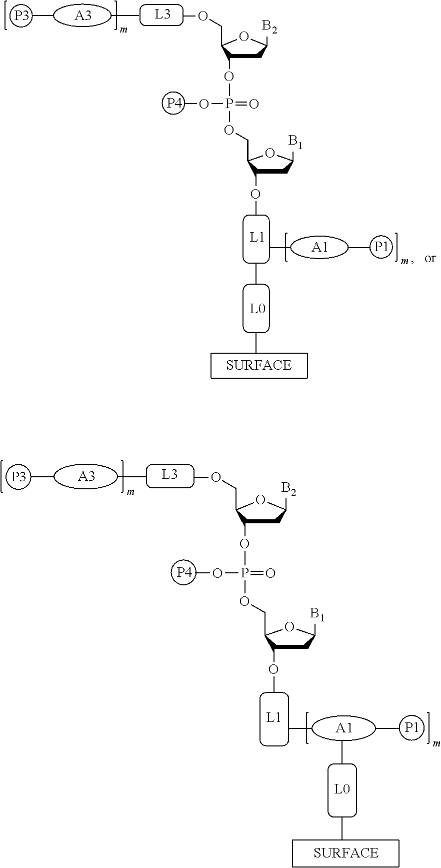

wherein:
   L1-A1-P1 together represents a safety catch linker for attachment at the 3′—OH group of the nucleoside to the surface, wherein:
      P1 represents a protecting group,
      L1 represents a thermally cleavable linker moiety,
      A1 represents an activator moiety that, upon removal of P1, is capable of causing cleavage of the cleavable linker from the solid surface;
   P3-A3-L3 together represents a safety catch 5-OH protecting group, wherein:
      P3 represents a protecting group,
      L3 represents a cleavable linker moiety,
      A3 represents an activator moiety that, upon removal of P3, is capable of causing removal of the 5′-OH protecting group;
   m at each occurrence is the same or different, and represents 1 or 2;
   L0 represents a moiety for attachment of the first nucleoside via the cleavable linker group to the surface;
   Each B₁ and B₂ independently represents an optionally protected canonical or an optionally protected non-canonical nucleobase,
wherein A1, A3, L1 and L3 can be the same or different, and wherein P1 and P3 are different and are removable under different conditions or reagents; and
   P4 represents a phosphoramidite protecting group.

Alternatively, the coupling of a di-nucleotide 3′-phosphoramidite comprising a 5′-OH protecting group in step (iii) to a deprotected 5′—OH group of the immobilized nucleoside, followed by oxidation, forms a structure represented by:

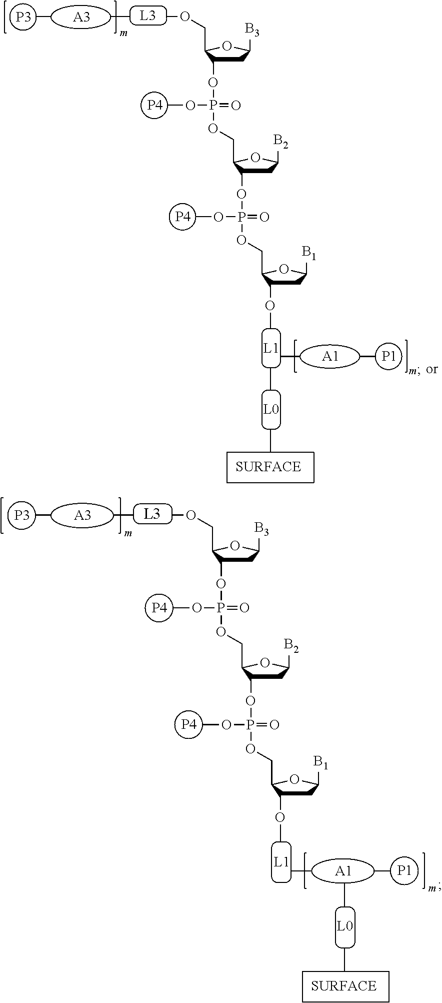

or wherein the coupling of a tri-nucleotide 3′-phosphoramidite comprising a 5′-OH protecting group in step (iii) to a deprotected 5′—OH group of the immobilized nucleoside, followed by oxidation, forms a structure represented by:

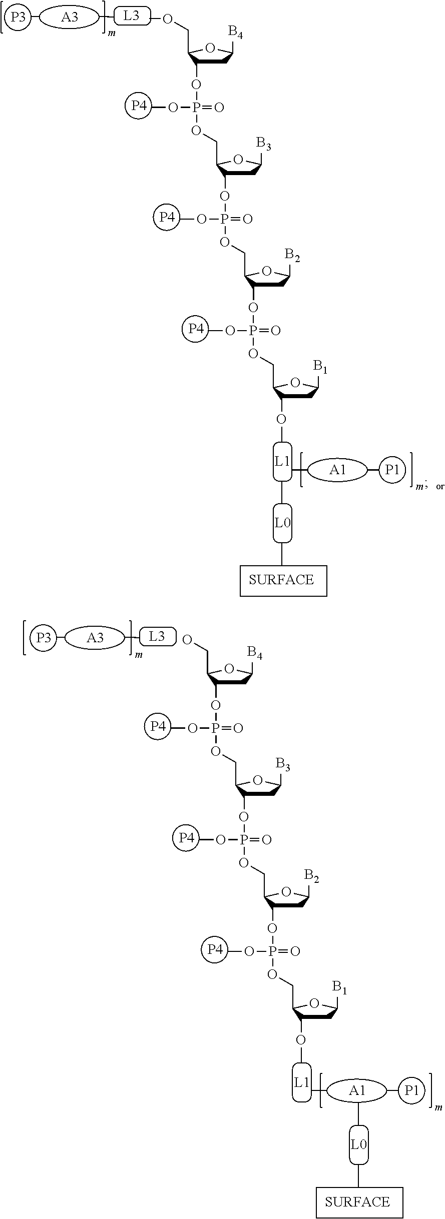

wherein:
   L1-A1-P1 together represents a safety catch linker for attachment at the 3′—OH group of the nucleoside to the surface, wherein:
      P1 represents a protecting group,
      L1 represents a thermally cleavable linker moiety,
      A1 represents an activator moiety that, upon removal of P1, is capable of causing cleavage of the cleavable linker from the solid surface;
   P3-A3-L3 together represents a safety catch 5′-OH protecting group, wherein:
      P3 represents a protecting group,
      L3 represents a cleavable linker moiety,
      A3 represents an activator moiety that, upon removal of P3, is capable of causing removal of the 5′-OH protecting group;
   m at each occurrence is the same or different, and represents 1 or 2;
   L0 represents a moiety for attachment of the first nucleoside via the cleavable linker group to the surface;
   each B₁, or B₂ or B₃ or B₄ independently represents an optionally protected canonical or an optionally protected non-canonical nucleobase,
wherein A1, A3, L1 and L3 can be the same or different, and wherein P1 and P3 are different and are removable under different conditions or reagents; and
   each P4 may be the same or different and each represents a phosphate protecting group.

Steps (ii) and (iii) are repeated to sequentially grow the oligonucleotides at each site by successive thermally controlled deprotection at the 5′-OH of the nucleosides and coupling of an incoming nucleoside represented by:

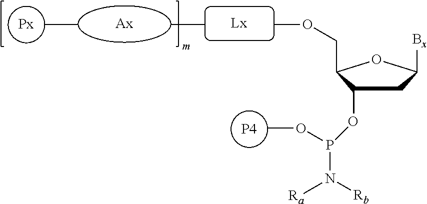

wherein:
   Px-Ax-Lx together represents a cleavable 5′-OH protecting group which protects the 5′—OH group of the incoming nucleoside, wherein:
      Lx represents a cleavable linker moiety,
      Px represents a protecting group, and
      Ax represents an activator moiety that, upon removal of Px, is capable of causing removal of the 5′-OH protecting group;
   m=1 or 2;
   P4 represents a phosphoramidite protecting group;
   Bₓ represents an optionally protected canonical or an optionally protected non-canonical nucleobase; and
   Rₐ and R_{b} can be the same or different and each represent alkyl.

Preferably, the nucleoside comprising a 5′-OH-protecting group in step (iii) and step (v) is a nucleoside 3′-phosphoramidite comprising a thermally cleavable 5′-OH protecting group, wherein after each coupling step, the resulting phosphite triester is converted to phosphate triester by oxidation. Oxidation of the phosphite can be achieved by iodine oxidation in the presence of water and a weak base, such as pyridine, lutidine, or collidine [see Matteucci, M. D.; Carruthers, M. H. (1981). “Synthesis of deoxyoligonucleotides on a polymer support”. J. Am. Chem. Soc. 103 (11): 3185] or using of tert-butyl hydroperoxide and (1S)-(+)-(10-camphorsulfonyl)oxaziridine.

Alternatively, steps (ii) and (iii) are repeated, to sequentially grow the oligonucleotides at each site by successive thermally controlled deprotection at the 5′-OH of the nucleosides/nucleotide and coupling of an incoming nucleotide represented by:

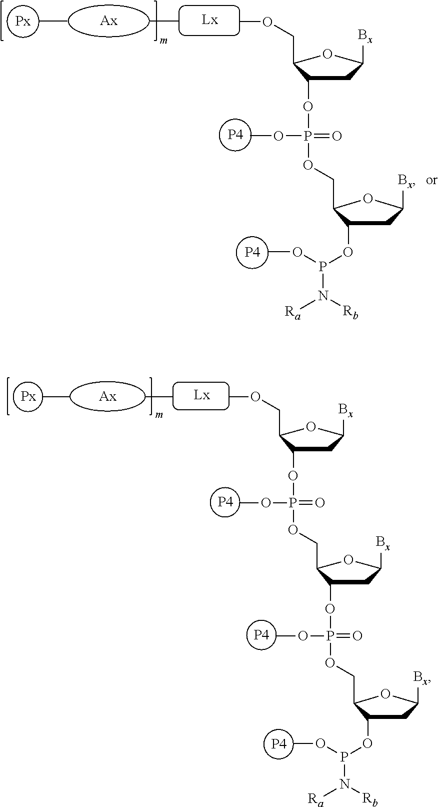

wherein:
   Px-Ax-Lx together represents a cleavable 5′-OH protecting group which protects the 5′—OH group of the incoming nucleoside or nucleotide, wherein:
      Lx represents a cleavable linker moiety,
      Px represents a protecting group, and
      Ax represents an activator moiety that, upon removal of Px, is capable of causing removal of the 5′-OH protecting group;
   m=1 or 2;
   each P4 can be the same or different and each represents a phosphoramidite or phosphate protecting group;
   each Bₓ may be the same or different and each independently represents an optionally protected canonical or an optionally protected non-canonical nucleobase; and
   Rₐ and R_{b} can be the same or different and each represents alkyl.

### 5′-Hydroxy Protecting Groups, Cleavable Linkers and Other Protecting Groups

The thermally controlled synthesis of oligonucleotide fragments from the surface-attached and protected first nucleoside or protected first nucleotide can be seen as a modified version of the phosphoramidite chemical cycle.

In the phosphoramidite method, a 5′-protected nucleoside is first covalently attached to a solid support, for example a polymer support. The 5′-protecting group (typically trityl in standard phosphoramidite synthesis) is removed and a nucleoside-3′-phosphoramidite is coupled to the 5′-hydroxyl group to form a support-bound phosphite triester. Optionally, the resulting product is treated with a capping agent, in order to remove failed sequences/unreacted nucleoside, typically by acetylation. The phosphite triester is then oxidised to the corresponding phosphotriester. The deprotection, coupling and oxidation steps are repeated until the desired oligonucleotide has been prepared. The resulting product is a support-bound oligonucleotide, which is then treated in order to release the oligonucleotide from the support, and subsequently separation of the oligonucleotide from the support, e.g. by filtration. The present invention employs a thermally cleavable 5′-OH protecting group for the starting first nucleoside or the starting nucleotide (e.g. di- or tri-nucleotide), as well as for the subsequent incoming nucleoside building blocks (e.g. nucleoside 3′-phosphoramidites, or nucleotide 3′-phosphoramidites), and advantageously, a thermally cleavable linker group for the attachment of the first nucleoside or first nucleotide at the 3′-OH to the substrate.

The cleavable linker between the support and the oligonucleotide must be stable to the oligonucleotide synthesis procedure, whilst being capable of facile release under thermal control when required, at the end of the synthesis.

The 5′-OH protecting group is preferably a safety-catch-type thermosensitive protecting group. The safety catch type system advantageously provides storage, transport and synthetic stability, and is susceptible to heat-induced cleavage only after a first, separate activation step.

Preferably, the 5′-OH protecting group is the same for each of the nucleoside or nucleotide building blocks (5′-OH protected nucleoside 3′-phosphoramidite, or 5′-OH protected nucleotide 3′-phosphoramidite).

Thus, the first nucleoside or nucleotide and the subsequent incoming nucleoside building blocks (e.g. nucleoside 3′-phosphoramidite, or nucleotide 3′-phosphoramidite reagents such as a di-nucleotide 3′-phosphoramidite or a tri-nucleotide 3′-phosphoramidite) are advantageously protected at the 5′-position with a thermosensitive safety-catch protecting group. The conditions for unlocking and removal of this protecting group are desirably orthogonal with the following chemical processes: removal of the phosphate protecting groups on the internucleosidic phosphate moieties; removal of the exocyclic nitrogen protecting groups required for bases such as adenine, cytosine and guanine; and unlocking and cleavage of the linker attaching the oligonucleotide fragments to the surface. The protecting group must also be stable to the conditions used in the phosphoramidite cycle which are: the mild acid-catalysed reaction of the phosphoramidite reagent with the 5′-hydroxy group on the growing oligonucleotide fragment; and subsequent oxidation of the newly made phosphite linkage to the desired phosphate. The heated or unheated conditions used for unlocking and deprotection should preferably not cause any more damage to the growing oligonucleotide fragments than can be considered negligible to the oligonucleotide fragment synthesis. The thermal control of the deprotection must be of a sufficient level so as to minimise misincorporation of nucleosides or nucleotides in the growing oligonucleotide fragments, either by unwanted deprotection at the cold site, or insufficient deprotection at the hot sites

The safety catch linker groups and protecting groups require a two step removal process. In a first step, i.e. the activation step, a protecting group PG is removed under a specific reaction condition to expose a deprotected activating group and a linker moiety. The second step, i.e. a cleavage step, involves a second reaction condition (temperature increase optionally in the presence of an acid or base) whereby the deprotected activating group causes intramolecular cyclisation with concomitant release of carbon dioxide. Hence the selected reaction sites that have been heated will then contain surface attachments with a deprotected moiety, which can then react with an incoming molecule comprising a complimentary reactive moiety attached to a 5′-hydroxy protected first nucleoside (or a 5′-hydroxy protected nucleotide, such as a 5′-hydroxy protected di-nucleotide or a 5′-hydroxy protected tri-nucleotide) via a thermally cleavable linker attached at the 3′-position. The linker group that attaches the first nucleoside or nucleotide to the surface is preferably likewise of the safety catch type. The protecting group PG should be stable to removal during the oligonucleotide synthesis process, i.e. the safety catch linker should be in its locked (protected) form until the end of the oligonucleotide synthesis. Upon unlocking, the surface will contain a plurality of oligonucleotides that are bound to the surface via unlocked, thermally cleavable linker groups. Heating specific sites will then cause the oligonucleotides at those sites to cleave from the surface, thereby enabling controlled release of the oligonucleotides (e.g. for hybridization).

The unlocking of the protecting group on the surface-attached reactive moiety can be carried out in either a thermally or non-thermally controlled fashion. Thermally controlled unlocking is required if the unlocked protecting group on the surface-attached reactive moiety is able to react with the reactive moiety on the incoming linker-attached first nucleoside or nucleotide. This is because, with no thermal control, all of the surface-attached protecting groups will be simultaneously unlocked. If the unlocked protecting group on the surface-attached reactive moiety cannot react with the reactive moiety on the incoming linker-attached first nucleoside (or nucleotide), thermal control is not required.

The conditions used for unlocking the 5′-OH protecting group on the surface-attached reactive moiety are required to be orthogonal to the conditions used for unlocking the thermally cleavable linker and the 5′-OH protecting group, but only at the cold temperature.

Thermally controlled synthesis of oligonucleotides or oligonucleotide fragments from the surface-attached and protected first nucleoside is achieved by first unlocking the protecting group on the first nucleoside (or nucleotide), such that it is susceptible to removal when subjected to the high temperature conditions. Reaction sites that have been heated will then contain nucleosides or (nucleotides) with a deprotected 5′-hydroxy group, which can then react with the incoming 5′-hydroxy protected nucleoside or 5′-hydroxy protected nucleotide.

The deprotected 5′-hydroxy group may react with the incoming nucleoside or nucleotide in any known way. Preferably, the incoming nucleoside or nucleotide is a nucleoside 3′-phosphoramidite reagent, a nucleotide 3′-phosphoramidite reagent (particularly a di-nucleotide 3′-phosphoramidite reagent, or a tri-nucleotide 3′-phosphoramidete reagent), and the coupling reaction is followed by oxidation of the newly made phosphite linkage to a phosphate group.

Preferably, the phosphoramidite reagent is a 3′-O—(N,N-dialkylphosphoramidite) [preferably 3′-O—(N,N-diisopropylphosphoramidite] derivative of a nucleoside or a nucleotide, protected at any exocyclic nitrogens, such as in the case of adenine, cytosine and guanine, and protected at the nucleophilic oxygen contained in the phosphite group, and protected at the 5′—OH group with a thermally cleavable protecting group as described herein.

Activation of the phosphoramidite for the coupling reaction may be carried out by adding a 0.2-0.7 M acetonitrile solution of an acidic molecule which can act as a weak acid to protonate the phosphoramidite, while also being a nucleophile which displaces the dialkylamino group. Examples of such reagents are tetrazole and its derivatives, for example, 4,5-Dicyanoimidazole (DCI), Ethylthiotetrazole (ETT), 5(4-nitrophenyl)-1H-tetrazole and 5-ethylthio-1H-tetrazole.

In any aspect of the present invention, the thermally cleavable linker group is represented by the formula (L-1):

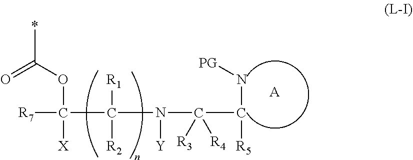

wherein:
   * represents a point of attachment to the 3′-OH of the nucleoside or nucleotide;
   X represents hydrogen or hydrocarbyl;
   Y represents hydrocarbyl or

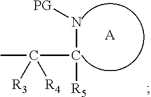

each of R₁, R₂, R₃, R₄, R₅ and R₇ are the same or different and each independently represents hydrogen or hydrocarbyl;
PG represents a cleavable protecting group for nitrogen;
n represents 0, 1, 2 or 3; and
ring A represents a nitrogen-containing heterocyclic group;
wherein at each occurrence R₁, R₂, R₃, R₄, R₅, PG and A may be the same or different, which is bound to the substrate at one of R₁, R₂, R₃, R₄, R₅, R₇, X, Y or A, preferably which is bound to the substrate at R₇ or Y, and preferably which is which is bound to the substrate at R₇ when Y is:

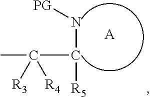

or wherein the cleavable linker is bound to the substrate at Y when Y is hydrocarbyl.

Preferably Y is hydrocarbyl.

Preferably, at least one of the protecting groups PG of the cleavable linker (L-1) is cleavable under a first reaction condition to produce a deprotected linker, wherein the deprotected linker can undergo intramolecular cyclisation and cleavage under thermal control with release of carbon dioxide under a second, different, reaction condition, to produce a compound of formula (II):

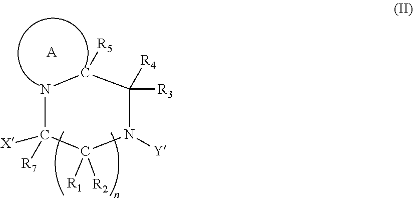

thereby releasing the oligonucleotide from the surface;
wherein PG′ is hydrogen or a cleavable protecting group for nitrogen, provided that at
least one PG′ is hydrogen;
   Y′ represents hydrocarbyl, or

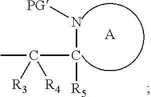

and
wherein X, R₁-R₅, R₇, A, and n are as defined above.

In any aspect of the present invention, the 5′-OH protecting group is represented by the formula (L-1′):

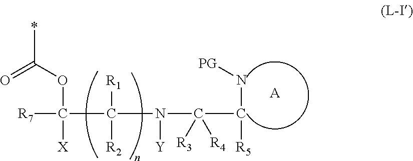

wherein:
   * represents a point of attachment to the 5′-OH of the nucleoside or nucleotide;
   X represents hydrogen or hydrocarbyl;
   Y represents hydrocarbyl or

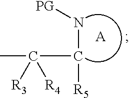

each of R₁, R₂, R₃, R₄, R₅ and R₇ are the same or different and each independently represents hydrogen or hydrocarbyl;
PG represents a cleavable protecting group for nitrogen which is different from the PG group in formula L-1;
n represents 0, 1, 2 or 3; and
ring A represents nitrogen-containing heterocyclic group;
wherein at each occurrence R₁, R₂, R₃, R₄, R₅, PG and A may be the same or different.

Preferably in the protecting group L-1′, at least one of the protecting groups PG is cleavable under a first reaction condition to produce a deprotected linker, wherein the deprotected linker can undergo intramolecular cyclisation and cleavage, under thermal control, with release of carbon dioxide under a second, different, reaction condition, to produce a compound of formula (II):

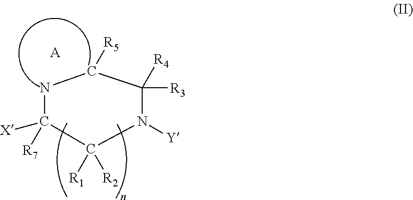

thereby deprotecting the 5′—OH group of the nucleoside or nucleotide;
wherein
   PG′ is hydrogen or a cleavable protecting group for nitrogen, provided that at least one PG′ is hydrogen;
   Y′ represents hydrocarbyl, or

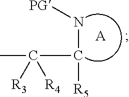

and
wherein X, R₁-R₅, R₇, A, and n are as defined above.
In formula L-1′, Y is preferably

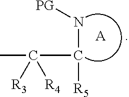

The protecting group of formula (L-I′) contains an activator group (ring A), which on heating in suitable conditions causes the molecule to fragment, leaving the 5′-hydroxy group on the growing oligonucleotide free to react with an incoming phosphoramidite reagent. The fragmentation reaction is highly thermally sensitive and occurs under mild conditions, since the activator and acceptor groups are tethered in close vicinity to each other. This means that no other reagents are required for the heated process, dramatically reducing the number of side-reactions that can occur while heat is applied.

The PG for the thermally cleavable protecting group on the first nucleoside or nucleotide must be orthogonal to any other protecting groups on the first nucleoside or nucleotide, and to the PG for the thermally cleavable linker. Preferably, the PG for the thermally cleavable linker is stable to the oligonucleotide synthesis reactions, since the PG for the cleavable linker is preferably removed only at the end of the oligonucleotide synthesis. Preferably, the PG for the thermally cleavable protecting group is an acid labile protecting group, such as Adpoc or Ddz.

The 5′-OH protected nucleoside 3′-phosphoramidite or 5′-OH protected nucleotide 3′-phosphoramidite preferably contains a 5′-OH protecting group of the safety catch type (formula L-1′). The PG on the 5′-OH protecting group may preferably be a base-labile protecting group such as Fmoc or Bsmoc.

For nucleosides or nucleotides having nucleobases that include exocyclic nitrogens, such as on adenine, cytosine and guanine bases, the exocyclic nitrogens may be protected by protecting groups that are orthogonal to the PG for the thermally cleavable linker. Preferably, the protecting groups for the exocyclic nitrogens are palladium-labile protecting groups, such as alloc, or fluoride-labile groups, such as those disclosed in WO2014/022839, Matteucci, M. D.; Carruthers, M. H. (1981)—“Synthesis of deoxyoligonucleotides on a polymer support, J. Am. Chem. Soc. 103 (11): 3185, bis-tert-butyliosbutylsilyl (BIBS) [Huan Liang, Lin Hu, and E. J. Corey—Org. Lett., 2011, 13 (15), pp 4120-4123]. Alternatively, the exocyclic nitrogens are not protected. In this case, the oligonucleotides may be synthesised using phosphoramidite chemistry and a “proton-blocking” strategy to prevent the exocyclic amines from reacting with the phosphoramidite reagent. The “proton-blocking” strategy involves using activator acids with suitably low pKa to protonate the exocyclic amines to such an extent that they are not able to act as nucleophiles towards the phosphoramidite. Activators used for this method include 5-nitrobenzimidazolium triflate and other analogues [Sekine, J. Org. Chem. 2003, 68, 5478].

The phosphate group on the incoming nucleoside (nucleoside 3′-phosphoramidite) or incoming nucleotide (nucleotide 3′-phosphoramidite) (e.g. P4) may be the same or different from the protecting group used for protecting the exocyclic nitrogens on the nucleobases. Preferably, a palladium-labile protecting group, such as Alloc, Noc, Coc or Prenyl carbamate may be used for protection of the phosphate/phosphoramidite. Other suitable P4 groups for protection of phosphate or phosphoramidite include fluoride-labile groups, such as trimethylsilylethyl.

The cleavable linker group of formula (L-1) attaching the first nucleoside or nucleotide and the oligonucleotide during the synthesis is preferably attached at the 3′-OH of the first nucleoside or nucleotide and is attached to the surface of the substrate at the other end. The cleavable linker group similarly contains an activator group (ring A), which on heating in suitable conditions causes the molecule to fragment, thereby detaching the oligonucleotide from the surface of the substrate. The fragmentation reaction is highly thermally sensitive and occurs under mild conditions, since the activator and acceptor groups are tethered in close vicinity to each other. This means that no other reagents are required for the heated process, dramatically reducing the number of side-reactions that can occur while heat is applied. Once the activator group on the thermally cleavable linker or the thermally cleavable protecting group (e.g. N) has been sufficiently neutralised the thermal cleavage can be effected by heating in the presence of a solvent.

The thermally cleavable linker can be bound to the surface of the substrate at any suitable position, for example at one of the R₁, R₂, R₃, R₄, R₅, R₇, X, Y or A groups. Preferably, the thermally cleavable linker is covalently bound to the surface at R₇ or Y, and more preferably at Y. In particular, the thermally cleavable linker is covalently bound to the surface at Y wherein Y is:

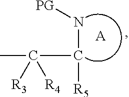

preferably via a linker group L0. Preferably, each R₁, R₁, R₂, R₃, R₄, R₅, PG and A is the same.

In any aspect of the invention, the thermally cleavable protecting group L-1′ or thermally cleavable linker of formula (L-1) preferably has the formula (L-IA) or (L-IB):

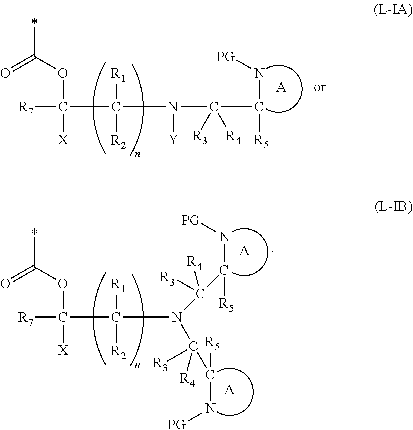

The phosphoramidite reagents and subsequent phosphates on the growing oligonucleotide are protected by protecting groups that are orthogonal to the PG for the thermally cleavable protecting group on the phosphoramidite reagent. Preferably, the protecting groups for the phosphoramidites are base-labile protecting groups, such as 2-cyanoethyl, palladium-labile protecting groups, such as allyl, or fluoride-labile groups, such as trimethylsilylethyl [see Wada, T.; Sekine, M. Tetrahedron Lett. 1994, 35, 757-760], 2-diphenylmethylsilylethyl [see (a) Hayakawa, Y.; Uchiyama, M.; Kato, H.; Noyori, R. Tetrahedron Lett. 1985, 26, 6505-6508. (b) Hayakawa, Y.; Kato, H.; Uchiyama, M.; Kajino, H.; Noyori, R. J. Org. Chem. 1986, 51, 2400-2402. (c) Hayakawa, Y.; Kato, H.; Nobori, T.; Noyori, R.; Imai, J. Nucleic Acids Res. Ser. 1986, 17, 97-100. (d) Hayakawa, Y.; Wakabayashi, S.; Kato, H.; Noyori, R. J. Am. Chem. Soc. 1990, 112, 1691-1696. (e) Hayakawa, Y.; Hirose, M.; Noyori, R. J. Org. Chem. 1993, 58, 5551-5555. (f) Hayakawa, Y.; Hirose, M.; Noyori, R. Nucleosides Nucleotides 1994, 13, 1337-1345. (g) Bergmann, F.; Kueng, E.; Laiza, P.; Bannwarth, W. Tetrahedron Lett. 1995, 51, 6971-6976]. Preferably, the protecting groups are palladium-labile protecting groups.

Preferably the protecting groups on the phosphate are stable during the deprotection/coupling cycles.

This compound of formula (I) contains an activator group (ring A), which on heating in suitable conditions causes the molecule to fragment, leaving the 5′-hydroxy group on the growing oligonucleotide fragment free to react with an incoming phosphoramidite reagent. The fragmentation reaction is highly thermally sensitive and occurs under mild conditions, since the activator and acceptor groups are tethered in close vicinity to each other. This means that no other reagents are required for the heated process, dramatically reducing the number of side-reactions that can occur while heat is applied. Furthermore, this activator group can itself be protected, thus locking the protecting group in an unreactive state. As discussed above for Phase I, the unlocking of the protecting group can be carried out in either a thermally or non-thermally controlled fashion. Thermally controlled unlocking is required if the unlocked protecting group is able to react with the incoming phosphoramidite reagent. This is because, with no thermal control, all of the surface-attached 5′-hydroxy protecting groups will be simultaneously unlocked. If the unlocked protecting group cannot react with the incoming phosphoramidite then thermal control is not required. In terms of the overall length of the process, it is advantageous not to have a thermally controlled unlocking step since a high level of thermal control of reactions is intrinsically linked with a longer reaction time. Therefore, the preferred activator group is sufficiently basic that it is predominantly in the protonated form, and is therefore non-nucleophilic, under the acidic conditions used to catalyse the phosphoramidite reaction.

The protecting groups on the internucleosidic phosphate moieties are preferably palladium-labile protecting groups, such as Alloc (allyloxycarbonyl), Noc (p-Nitrocinnamyloxycarbonyl), Coc (cinnamyloxycarbonyl) or Prenyl carbamate. In addition, since the exocyclic nitrogen protecting groups require removal at the same time, i.e. after oligonucleotide fragment synthesis is complete, but before cleavage from the surface, the preferred unlocking conditions are orthogonal to the combined removal of both. The exocyclic nitrogen protecting groups used in conventional phosphoramidite oligonucleotide fragment synthesis are removed under strongly basic conditions, which also cleave the proposed linker. Therefore, the preferred exocyclic nitrogen protecting groups are protecting groups that are removed under the same conditions as the phosphate protecting groups (e.g. palladium-labile protecting groups, such as Alloc, Noc, Coc or Prenyl carbamate). Hence, the preferred unlocking step is carried out under non-basic conditions that do not cause any premature removal of either the phosphate or exocyclic protecting groups. Therefore, suggested protecting groups for the lock could be cleaved under, for example, moderately acid conditions that do not cause significant depurination (such as Adpoc (1-(1-Adamantyl)-1-methylethoxycarbonyl,) Ddz (α,α-dimethyl-3.5-dimethoxybenzyloxycarbonyl) or similar protecting groups). The most preferred unlocking step is carried out under acidic conditions.

In any aspect or embodiment of the present invention, ring A can be the same or different at each occurrence, and each represents a heterocyclic group as defined above. More preferably, ring A represents a 4-12 membered mono-, bi- or tri-cyclic, preferably mono- or bicyclic nitrogen-containing heterocyclic group, and which may contain, in addition to the nitrogen, one or more other heteroatoms selected from N, O or S, preferably O or N. Preferably, ring A represents a 4 to 8-membered monocyclic heterocyclic group. More preferably, ring A represents a 5, 6, or 7-membered monocyclic heterocyclic group. In other preferred embodiments, ring A represents a heterocycle selected from: piperidyl, morpholinyl, pyrrolidinyl, thiomorpholinyl, and imidazolyl. Even more preferably, ring A represents piperidyl, pyrrolidinyl or imidazolyl. In especially preferred embodiments of the present invention, ring A represents piperidyl, or pyrrolidinyl.

In any aspect or embodiment of the present invention, at each occurrence of —C(R₃)(R₄), both of R₃ or R₄ is hydrocarbyl, or one of R₃ or R₄ is hydrocarbyl, and the other is H, or both R₃ and R₄, represent H. Preferably, one of R₃ or R₄ is hydrocarbyl, and the other is H, or both R₃ and R₄, represent H.

In any aspect or embodiment of the present invention, n represents 0, 1 or 2; and preferably 0 or 1. Most preferably, n represents 1.

In any aspect or embodiment of the present invention, the group X is H or hydrocarbyl, wherein the hydrocarbyl is selected from the group consisting of alkyl, aryl or arylalkyl as defined above. Preferably X is H or aryl, and more preferably X is H or phenyl.

In any aspect or embodiment of the present invention, preferably the group R₇ is H.

In any aspect or embodiment of the present invention, preferable the groups R₁ and R₂ are preferably H.

In any aspect or embodiment of the present invention, preferably the groups R₃ and R₄ are H.

In any aspect or embodiment of the present invention, preferably the group R₅ is H.

In any aspect or embodiment of the present invention, the activation step, whereby at least one of the protecting groups PG is cleaved, is preferably effected by a change in pH, temperature, radiation, or by a chemical activating agent, or by a combination thereof. Preferably, the cleavage of at least one protecting group PG can be activated by pH, temperature, a chemical activation agent, or by a combination thereof.

In a preferred embodiment, at least one protecting group PG is thermally cleavable in the presence of an activating agent. Typically, at least one protecting group PG is not thermally cleavable in the absence of an activating agent. Preferably, the activating agent is an acid or a base. In accordance with any aspect or embodiment of the present invention, the conditions whereby the PG group can be cleaved are different from the conditions that effect intramolecular cyclisation to cleave the thermally cleavable linker or protecting group. The protecting groups can be selected in order to enable the two different conditions for activation and release.

In one embodiment, at least one protecting group PG is thermally cleavable in the presence of an acid, and the intramolecular cyclisation and cleavage of the linker is effected by heating in the presence of a base. In this embodiment, an acid-cleavable protecting group PG leads to deprotection of the PG group(s) resulting in a N-protonated intermediate. The N-protonated intermediate is then unable to effect linker cleavage until the deprotonation occurs, i.e. by reaction with a base. In a solution phase process deprotonation can be carried out with, for example, a cold aqueous basic work-up, before carrying out the second step of the process in a mild buffer. Alternatively the base used for the second step of the process can be basic enough to both effect the deprotonation and to facilitate the intramolecular cyclisation and cleavage of the linker. In a solid phase process an excess of organic base strong enough to deprotonate the N-protonated intermediate can be added to the buffer solution used for the second step, or a more basic buffer can be used. Thus the use of an acid-cleavable protecting group offers a different level of orthogonality in each of the deprotection (i.e. PG removal) and cleavage (intramolecular cyclisation) steps. Preferably, pH at which the acid-cleavable protecting group PG is removed (pH₁, wherein pH₁ is <7) and the pH at which the base-mediated intramolecular cyclisation is effected (pH₂, wherein pH₂ is >7) differs by: at least about 1, at least about 2, at least about 3, at least about 4, at least about 5, at least about 6, at least about 7, at least about 8, at least about 9, or at least about 10 pH units. Preferably, the difference in pH at which the protecting group PG and the intramolecular cyclisation/cleavage of the linker is: about 2 to about 10, about 3 to about 7, about 3 to about 7 or about 4 to about 7 or about 5 to about 6 pH units.

Preferred PG groups that are cleavable in the presence of acid are selected from: tert-butyloxycarbonyl (Boc), trityl (Trt), benzyloxycarbonyl, α,α-dimethyl-3,5-dimethoxybenzyloxycarbonyl (Ddz), 2-(4-biphenyl)isopropoxycarbonyl (Bpoc), 2-nitrophenylsulfenyl (Nps), tosyl (Ts). More preferably acid cleavable protecting groups are selected from Boc and Trt.

Alternatively, in another embodiment, at least one protecting group PG is thermally cleavable in the presence of a base (e.g. at a temperature T₁), and the intramolecular cyclisation and cleavage of the linker is effected by further heating (e.g. at a temperature T₂). The difference in temperatures (i.e. T₂−T₁), may be: at least about 20° C., at least about 25° C., at least about 30° C., at least about 35° C., at least about 40° C., at least about 45° C., at least about 50° C., at least about 55° C., at least about 60° C., at least about 65° C., at least about 70° C. or at least about 75° C. Preferably, the difference in temperatures at which the protecting group PG and the intramolecular cyclisation/cleavage of the linker occurs is from: about 30° C. to about 100° C., about 40° C. to about 90° C., about 50° C. to about 80° C. or about 55° C. to about 75° C.

Preferred PG groups that are cleavable in the presence of a base are selected from: (1,1-dioxobenzo[b]thiophene-2-yl)methyloxycarbonyl (Bsmoc), 9-fluorenylmethoxycarbonyl (Fmoc), (1,1-dioxonaphtho[1,2-b]thiophene-2-yl)methyloxycarbonyl (α-Nsmoc), 2-(4-nitrophenylsulfonyl)ethoxycarbonyl (Nsc), 2,7-di-tert-butyl-Fmoc, 2-fluoro-Fmoc, 2-monoisooctyl-Fmoc (mio-Fmoc) and 2,7-diisooctyl-Fmoc (dio-Fmoc), 2-[phenyl(methyl)sulfonio]ethyloxycarbonyl tetrafluoroborate (Pms), ethanesulfonylethoxycarbonyl (Esc), 2-(4-sulfophenylsulfonyl)ethoxycarbonyl (Sps), acetyl (Ac), benzoyl (Bz), CF₃C(═O)-trifluoroacetamido, and preferably wherein the base cleavable protecting group is selected from Bsmoc, Fmoc, α-Nsmoc, mio-Fmoc, dio-Fmoc, and more preferably Bsmoc.

In any aspect or embodiment of the present invention, PG is preferably selected from the group consisting of Boc, Fmoc or Bsmoc.

In another embodiment of the present invention, PG can be a cleavable protecting group, which is preferably cleavable in the presence of a palladium catalyst and an allyl scavenger, preferably wherein PG is Alloc (allyloxycarbonyl).

In accordance with any aspect or embodiment of the present invention, the group Y is preferably hydrocarbyl as described above. Preferably, the invention encompasses compounds wherein at least one Y group is hydrocarbyl, wherein at least one Y is alkyl, alkenyl, aryl, aralkyl, alkaryl, wherein said alkyl, alkenyl, aryl, aralkyl or alkaryl group is substituted with a terminal alkynyl group. The terms alkyl, alkenyl, aryl, aralkyl, alkaryl, and alkynyl are as defined. Particularly, in this embodiment, at least one Y group is alkyl, alkenyl, aryl, aralkyl, alkaryl, which is substituted with a terminal alkynyl group, wherein the terminal alkyne group is a C₂ to C₆ alkynyl group, more preferably a C₂ to C₄ alkynyl group, and most preferably ethynyl. In another embodiment, at least one Y group is aralkyl which is substituted with an alkynyl group and more preferably wherein one Y group is CH₂—(C₆H₄)CH≡CH.

The cleavable protecting groups and cleavable linker used in the invention comprise at least one of the protecting groups PG which is cleavable under a first reaction condition to produce a deprotected linker, wherein the deprotected linker can undergo intramolecular cyclisation and cleavage with release of carbon dioxide under a second, different, reaction condition, to produce a compound of formula (II):

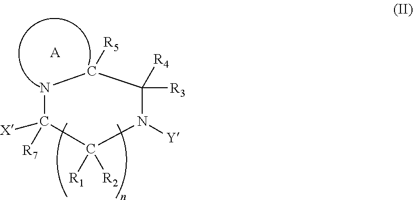

thereby releasing the organic moiety from the cleavable linker.

Preferably, in the cleavable protecting group or cleavable linker used in the invention, X is H or hydrocarbyl selected from the group consisting of alkyl, aryl or arylalkyl, preferably wherein X is aryl, and more preferably wherein X is phenyl, wherein alkyl, aryl or arylakyl are as defined above. Preferably, Y is benzyl. Alternatively, Y may be:

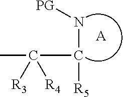

wherein each R₃, R₄ and R₅ represents hydrogen, both protecting groups PG are the same and both ring A are the same. Y is preferably

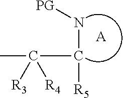

in the 5′-OH protecting groups. Y is preferably hydrocarbyl for the cleavable linkers.

In a preferred embodiment the ring A of the cleavable protecting group or cleavable linker, represents piperidinyl or pyrrolidinyl.

Preferred cleavable linkers are selected from the group consisting of: (L-IA) or (L-IB):

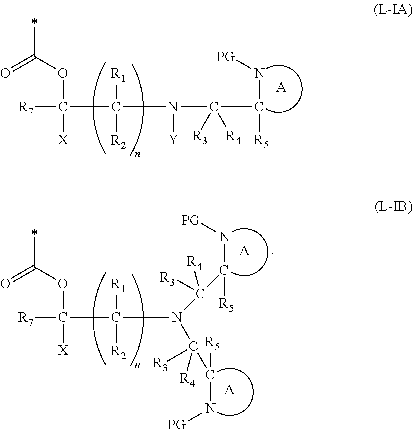

For the thermally cleavable linker group, L-IA is preferred, especially where Y is hydrocarbyl.

For the thermally cleavable 5′-OH protecting group (L-IB) is preferred.

Preferably, in the above cleavable protecting group or cleavable linker, at least one of the protecting groups PG is cleavable under a first reaction condition to produce a deprotected linker, wherein the deprotected linker can undergo intramolecular cyclisation and cleavage with release of carbon dioxide under a second, different, reaction condition, to produce a corresponding compound of formula (IIA) and (IIB) respectively:

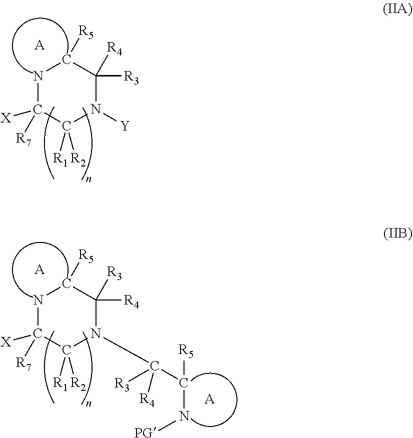

wherein PG′ in (IIB) is hydrogen or a cleavable protecting group for nitrogen, thereby deprotecting the oligonucleotide or releasing the oligonucleotide from the surface.

Preferably PG′ in compound (IIB) is hydrogen.

Preferably, ring A represents a 4-12 membered mono-, bi- or tri-cyclic, preferably mono- or bicyclic nitrogen-containing heterocyclic group, and which may contain, in addition to the nitrogen, one or more other heteroatoms selected from N, O or S, preferably O or N. More preferably, ring A represents a 4 to 8-membered monocyclic heterocyclic group. Particularly, ring A represents a 5, 6, or 7-membered monocyclic heterocyclic group.

Specifically preferred groups for ring A are heterocyclic groups selected from the group consisting of piperidyl, morpholinyl, pyrrolidinyl, thiomorpholinyl, and imidazolyl. In a further preferred embodiment, ring A represents piperidyl, pyrrolidinyl or imidazolyl. Most preferably, ring A represents piperidyl, or pyrrolidinyl.

In a preferred embodiment, the above cleavable protecting group or cleavable linker comprises R₃ and R₄ groups which are all H.

Preferably, n in the cleavable protecting group or cleavable linker according to any aspect of the present invention, n is 0, 1 or 2, preferably n is 0 or 1, and most preferably n is 1.

In a preferred embodiment of the cleavable protecting group or cleavable linker X is H or hydrocarbyl, wherein the hydrocarbyl is selected from the group consisting of alkyl, aryl or arylalkyl, wherein alkyl, aryl and arylkyl are described above. Preferably X is aryl, and more preferably X is phenyl.

In a preferred embodiment of the cleavable protecting group or cleavable linker R₅ is preferably hydrogen.

In a preferred embodiment of the cleavable protecting group or cleavable linker, X is: H or hydrocarbyl, wherein the hydrocarbyl is selected from the group consisting of alkyl, aryl or arylalkyl, as defined above, preferably wherein X is aryl, and more preferably wherein X is phenyl.

In a preferred embodiment of the cleavable protecting group or cleavable linker R₇ is H.

In a preferred embodiment of the cleavable protecting group or cleavable linker of this aspect of the present invention, R₁ and R₂ are H.

In a preferred embodiment of the cleavable protecting group or cleavable linker of this aspect of the present invention, R₃ and R₄ are both hydrogen.

The compounds used in the present invention can be prepared by the processes described below. The processes enable an efficient and facile preparation of compounds having a wide variety of different protecting groups PG. As discussed above, the use of different protecting groups enables fine control of the activation and cleavage step, ensuring that the linker group is only activated and subsequently released under specific reaction conditions.

As set out below, processes for the preparation of the compounds used in the present invention have been developed to enable the convenient modification of the protecting group PG from a common intermediate. The processes start from a heterocyclic compound containing a ketone or protected alcohol (see following Schemes 11-13). Use of a ketone substituted heterocyclic compound enables the preparation of compounds used in the invention wherein one of the R₃ or R₄ substituents is hydrocarbyl and the other is hydrogen, or wherein both R₃ and R₄ are hydrogen. Compounds wherein both R₃ and R₄ are hydrocarbyl can be prepared from the heterocyclic starting material containing a protected tertiary alcohol.

The starting heterocyclic compounds containing a ketone can be prepared in two steps by Grignard reaction of the corresponding Weinreb amide. The Weinreb amide can be prepared by reaction of the corresponding carboxylic acid with N,O-dimethylhydroxylamine hydrochloride in the presence of a peptide coupling reagent such as BOP [benzotriazol-1-yloxy)tris(dimethylamino)phosphonium hexafluorophosphate] or EDCI [1-Ethyl-3-(3-dimethylaminopropyl)carbodiimide] [see Nahm, S.; Weinreb, S. M. (1981), “N-methoxy-n-methylamides as effective acylating agents”, Tetrahedron Letters, 22: 3815, doi:10.1016/s0040-4039(01)91316-4], followed by reaction with a suitable Grignard reagent, such as an alkyl magnesium bromide (e.g. methyl magnesium bromide), as depicted in the below:

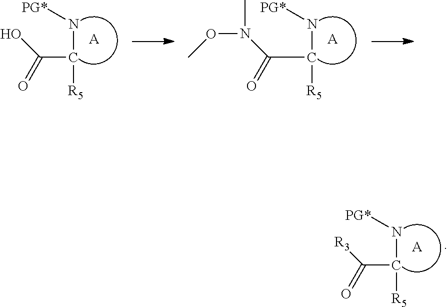

The starting material is protected at the ring nitrogen with a suitable protecting group PG*. The protecting group PG* can correspond to the PG protecting group in the final compound if appropriate. However, typically the protecting group PG* is selected such that it can be removed and replaced with the desired protecting group PG in the final compound or composition as shown in the schemes below.

In particular, the PG* protecting group should be stable to the subsequent coupling reaction wherein the starting nucleoside or nucleotide is coupled to the cleavable linker. The PG* protecting group should not be labile to conditions employed in the subsequent coupling reaction. Typically the coupling reaction to form the compound comprising the starting nucleoside or nucleotide bound to the cleavable linker is conducted in basic conditions. Hence, the PG* protecting group is preferably not labile to basic conditions. For example, the PG* protecting group may preferably be Boc or Alloc.

For compounds wherein R₃ and R₄ are both hydrocarbyl, heterocyclic starting material containing a tertiary alcohol is employed. The ring nitrogen is protected with the protecting group PG*, and subsequently, the hydroxyl group is derivatised to a suitable leaving group, for example tosylate or mesylate:

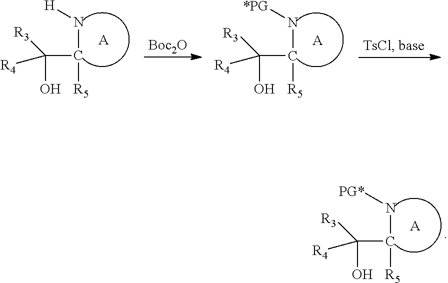

The following schemes describe preferred processes for preparing thermally cleavable linkers and protecting groups for use in the present invention.

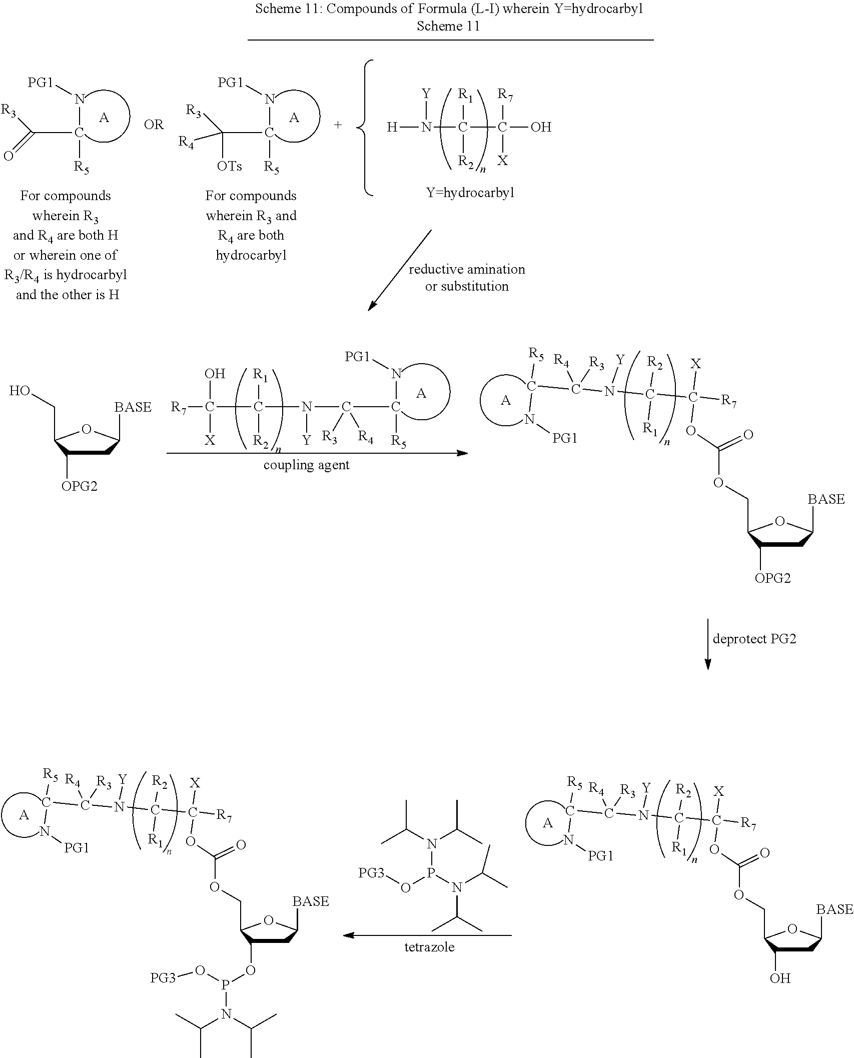

Scheme 11 above illustrates the synthesis of compounds containing a single activating group (PG) wherein Y is hydrocarbyl. The synthesis comprises a reductive amination or substitution reaction of the heterocyclic starting material containing a ketone or protected alcohol (depending on the R₃/R₄ substituent in the final compound), with the amine alcohol.

The resulting compound from the reductive amination or substitution reaction is then coupled to the nucleoside or nucleotide, suitably protected at the 3′-position, using a coupling agent [e.g. preferably using a 1,1′-carbonyldiimidazole (CDI)/1,8-diazabicyclo[5.4.0]undec-7-ene (DBU) coupling system]. The 3′-protecting group is then removed and the compound is converted into the phosphoramidite reagent.

Scheme 12: Compounds of Formula (L-I) wherein Y=

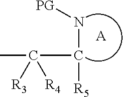

and R₃₋₅ PG and A are the same

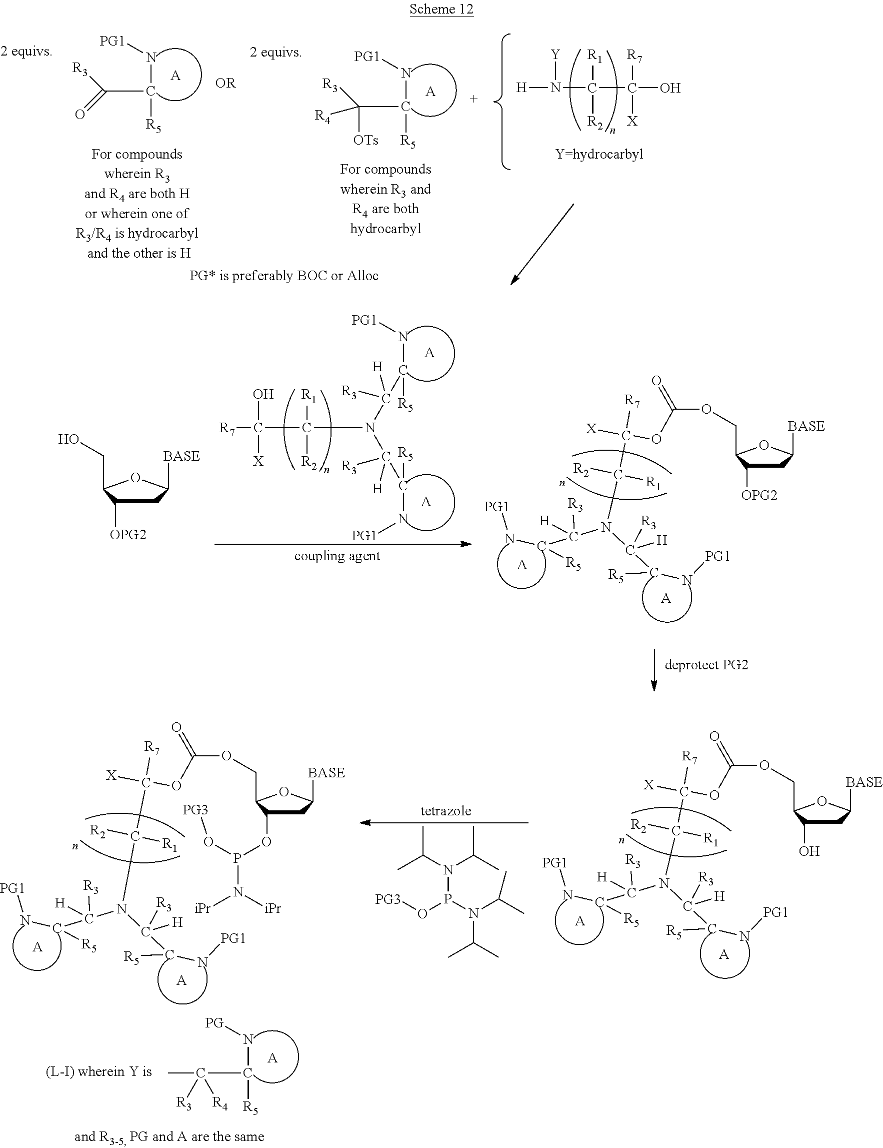

Scheme 12 above illustrates the synthesis of compounds of Formula L-I, containing two activating groups (PG) wherein Y is

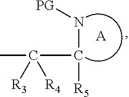

and R₃, R₅, PG and A are the same.

The synthesis comprises a reductive amination or substitution reaction of the heterocyclic starting material containing a ketone or protected alcohol (depending on the R₃/R₄ substituent in the final compound), with the amine alcohol. The reductive amination or substitution is carried out with two equivalents of the heterocyclic starting material.

The resulting compound from the reductive amination or substitution reaction is then coupled to the nucleoside or nucleotide, suitably protected at the 3′-position, using a coupling agent [e.g. preferably using a 1,1′-carbonyldiimidazole (CDI)/1,8-diazabicyclo[5.4.0]undec-7-ene (DBU) coupling system]. The 3′-protecting group is then removed and the compound is converted into the phosphoramidite reagent.

Scheme 13: Compounds of Formula (L-I) wherein Y=

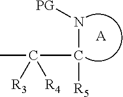

and R₃₋₅ PG and A are different

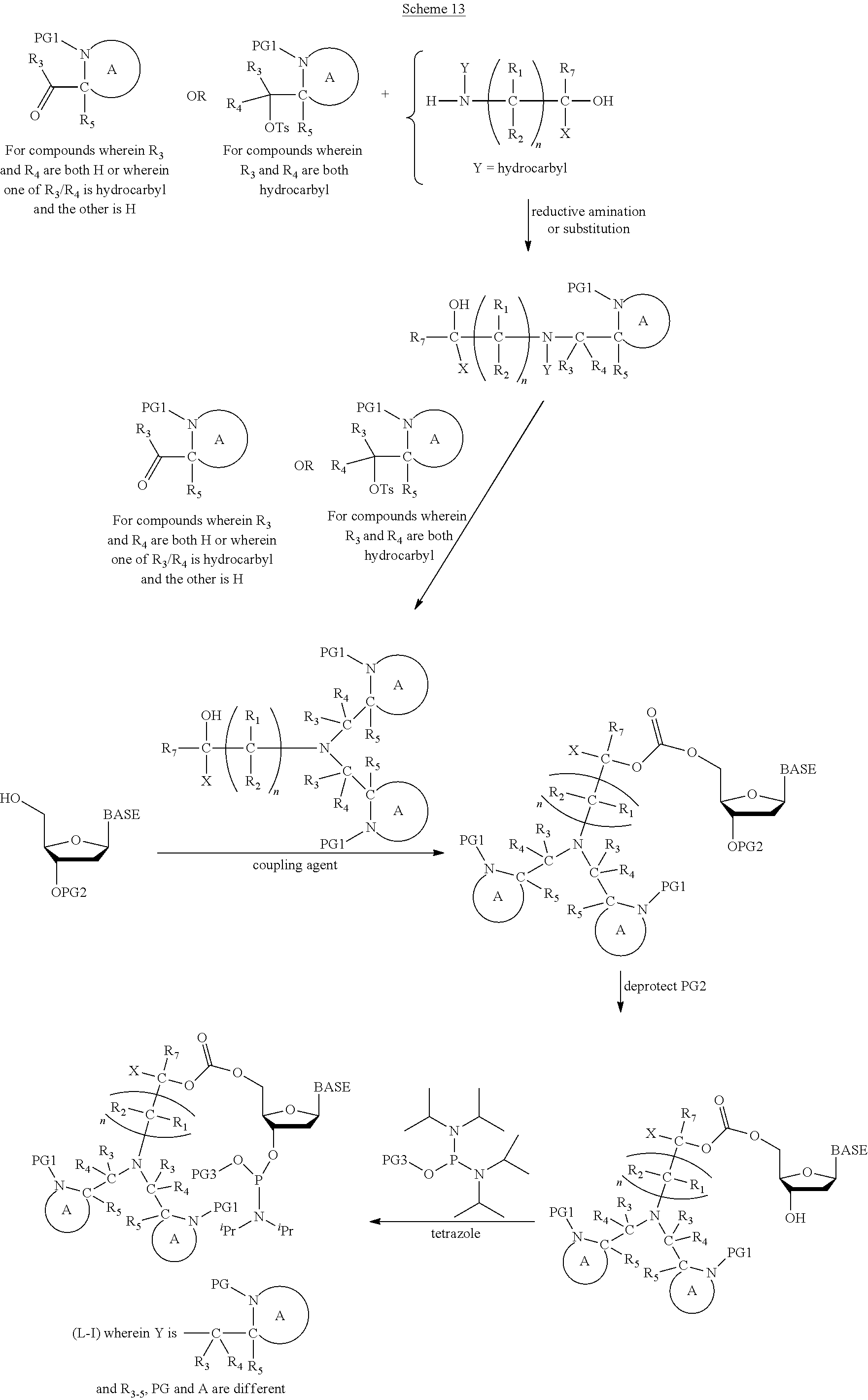

Scheme 13 above illustrates the synthesis of compounds of Formula L-I, containing two activating groups (PG) wherein Y is

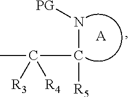

and R₃, R₅, PG and A are different.

The synthesis comprises a reductive amination or substitution reaction of the heterocyclic starting material containing a ketone or protected alcohol (depending on the R₃/R₄ substituent in the final compound), with the amine alcohol.

The heterocyclic starting material is protected at the ring nitrogen with a suitable protecting group PG1 (preferably BOC or Alloc, which are stable to the subsequent coupling reaction).

The resulting compound is subjected to a second reductive amination or substitution step using a heterocyclic starting material containing a ketone or protected alcohol as in the first step, but wherein the protecting group PG1 is different (e.g. the other of BOC or Alloc).

Coupling of the resulting compound with the nucleoside or nucleotide results in a compound containing two different PG protecting groups. The 3′-protecting group is then removed and the compound is converted into the phosphoramidite reagent.

The above processes can be modified by the appropriate derivatisation and incorporation of suitable functional groups in order to, for example, enable attachment of the thermally cleavable linker to the surface. In addition, the surface may be derivatized with suitable functional groups in order to enable its attachment to the thermally cleavable linker or to a reactive moiety.

Scheme 13A below illustrates the synthesis of the dimer phosphoramidite reagents (i.e. di-nucleotide 3′-phosphoramidites) comprising a 5′-OH protecting group, which can be used in steps (iii) and/or (v) of the oligonucleotide synthesis process of the present invention:

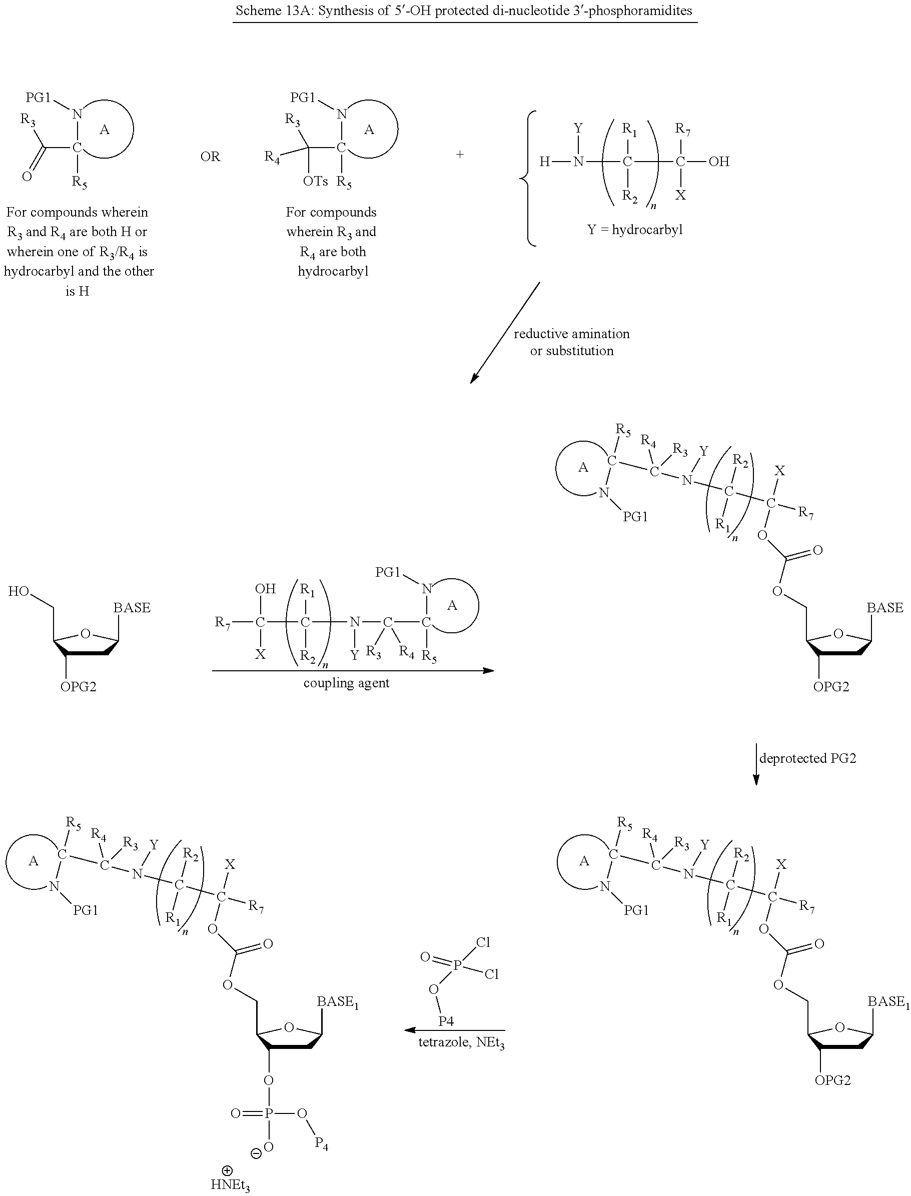

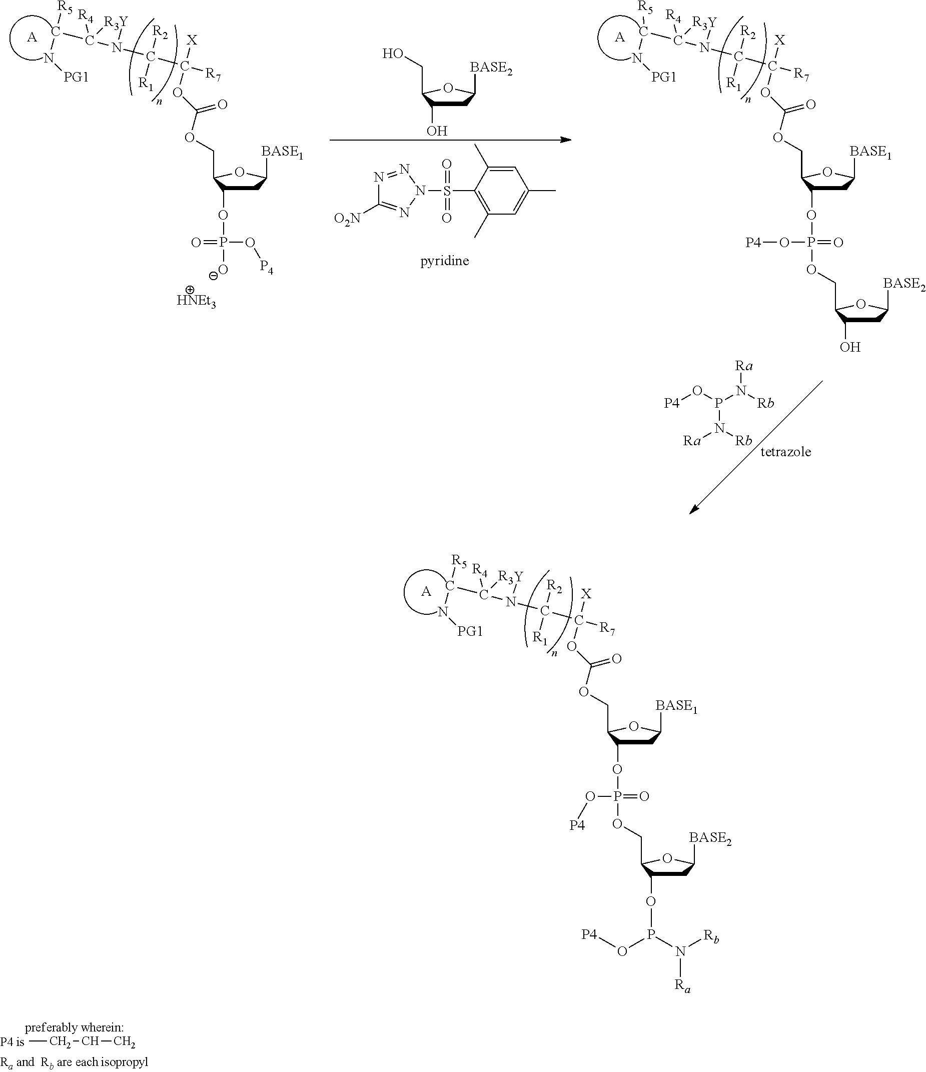

In the above Scheme 13A, the protecting group P4 on the phoshoramidite/phosphate is preferably a palladium-labile protecting group, such as allyl. Thus, following deprotection of the PG2 group, the compound may be reacted with e.g. dichlorophosphoric acid allyl ester to form the allylphosphate triethylammonium salt by reaction with the therosensitively protected base. Reaction of the resulting phosphoramidite with the base in the presence of 1-mesitylenesulfonyl-3-nitro-1,2,4-triazole (MSN) provides the di-nucleotide, which is converted to the phosphoramidite by reaction with e.g. an allyloxy[bis(dialkylamino)]phosphine (preferably allyloxy[bis(diisopropylamino)]phosphine). The trimers (5′-OH protected tri-nucleotide 3′-phosphoramidites may be similarly carried out by conducting two internucleotide linkage reactions followed by conversion to the phosphoramidite.

Attachment of the thermally cleavable linker to the surface can be achieved by the surface attachment means described above.

For the linker attachment, the appropriate thermally cleavable linker is prepared, wherein the linker fragment is provided with an alkynyl substituent at an appropriate position by use of an appropriately substituted starting material in accordance with the above reaction schemes. As stated above, the substrate can be attached to the linker via any of the substituent groups. For example, the substrate may be attached at any one of the R₁, R₂, R₃, R₄, R₅, R₇, X, Y or A positions.

The following reaction scheme (Scheme 14) illustrates process which can be used for the addition of the 5′-hydroxy protected first nucleoside or nucleotide, with a cleavable linker attached at the 3′-position, to the surface. The process can be readily modified to suit different 5′-hydroxy protecting groups, and different nucleosides or nucleotides, or nucleobases.

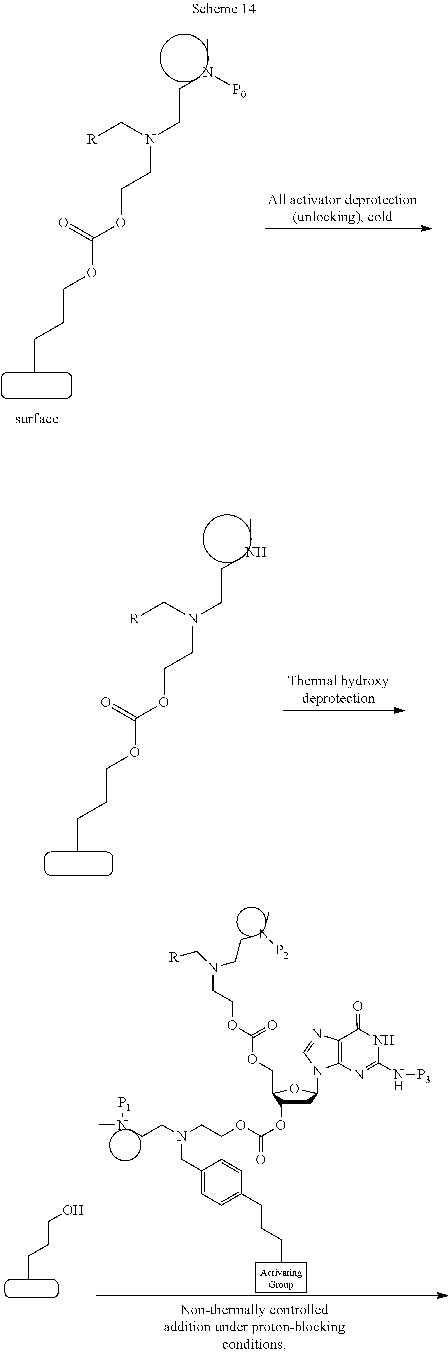

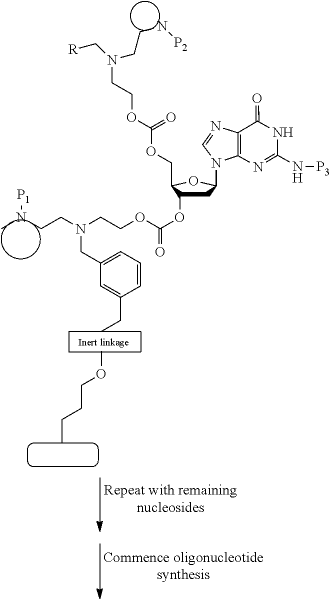

The following reaction scheme (Scheme 15) illustrates a process for the preparation of a 5′-hydroxy protected first nucleoside or nucleotide with a reactive moiety attached via a thermally cleavable linker attached at the 3′-position, from starting materials that are commercially available or the synthesis of which is known in the literature. The process can be readily modified to suit different 5′-hydroxy protecting groups, and different nucleosides, or nucleotides, or nucleobases:

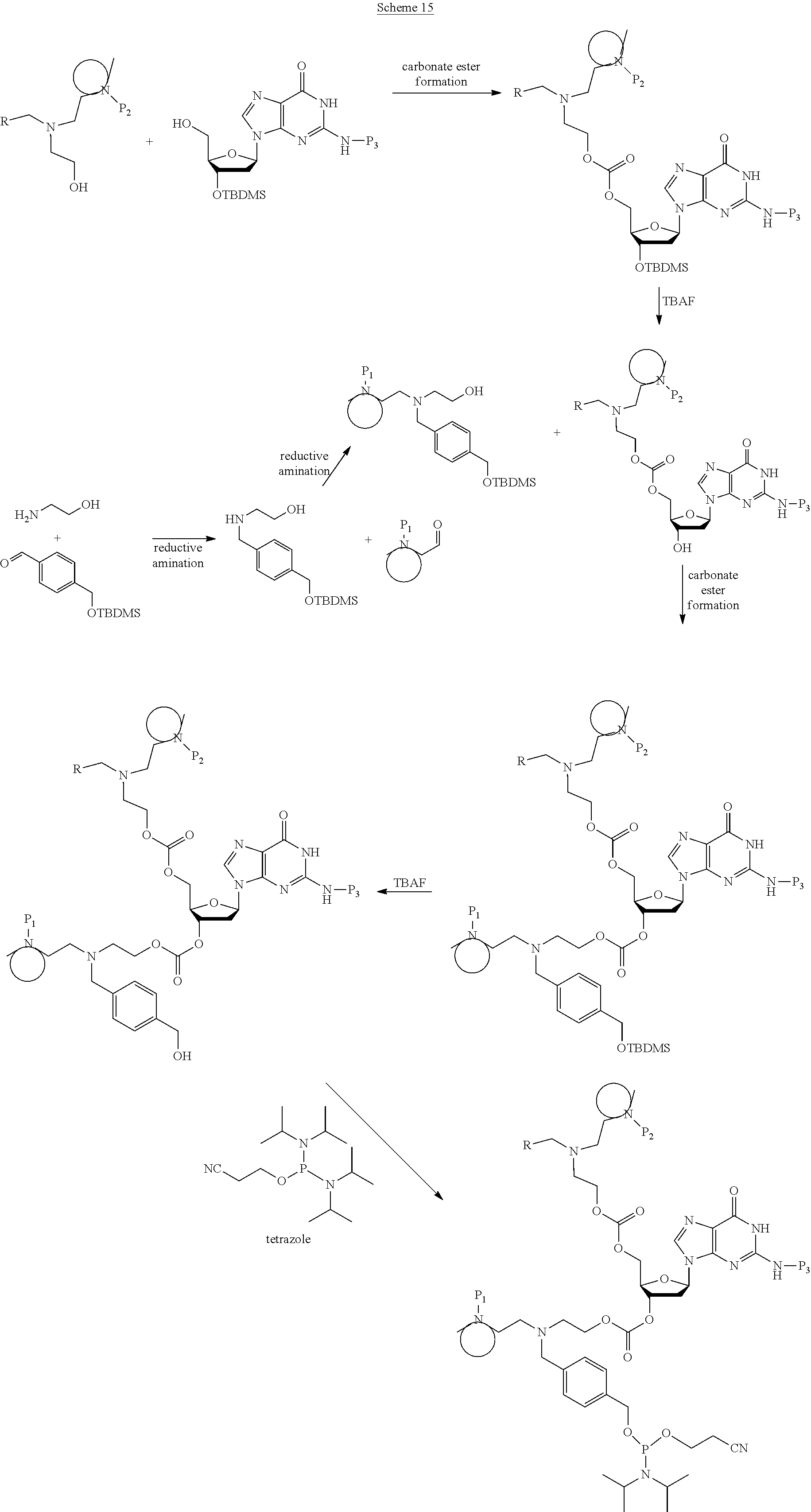

The following reaction scheme (Scheme 16) illustrates a process for the addition of a nucleoside building block (preferably a 5′-OH protected nucleoside 3′-phosphoramidite) to the growing oligonucleotide, followed by a penultimate deprotection of the nucleobase protection and phosphate protection, and finally thermal unlocking of the cleavable linker group, prior to linker cleavage. Cleavage of the linker under thermal control results in a selective and clean separation of the oligonucleotide at predetermined sites of the substrate.

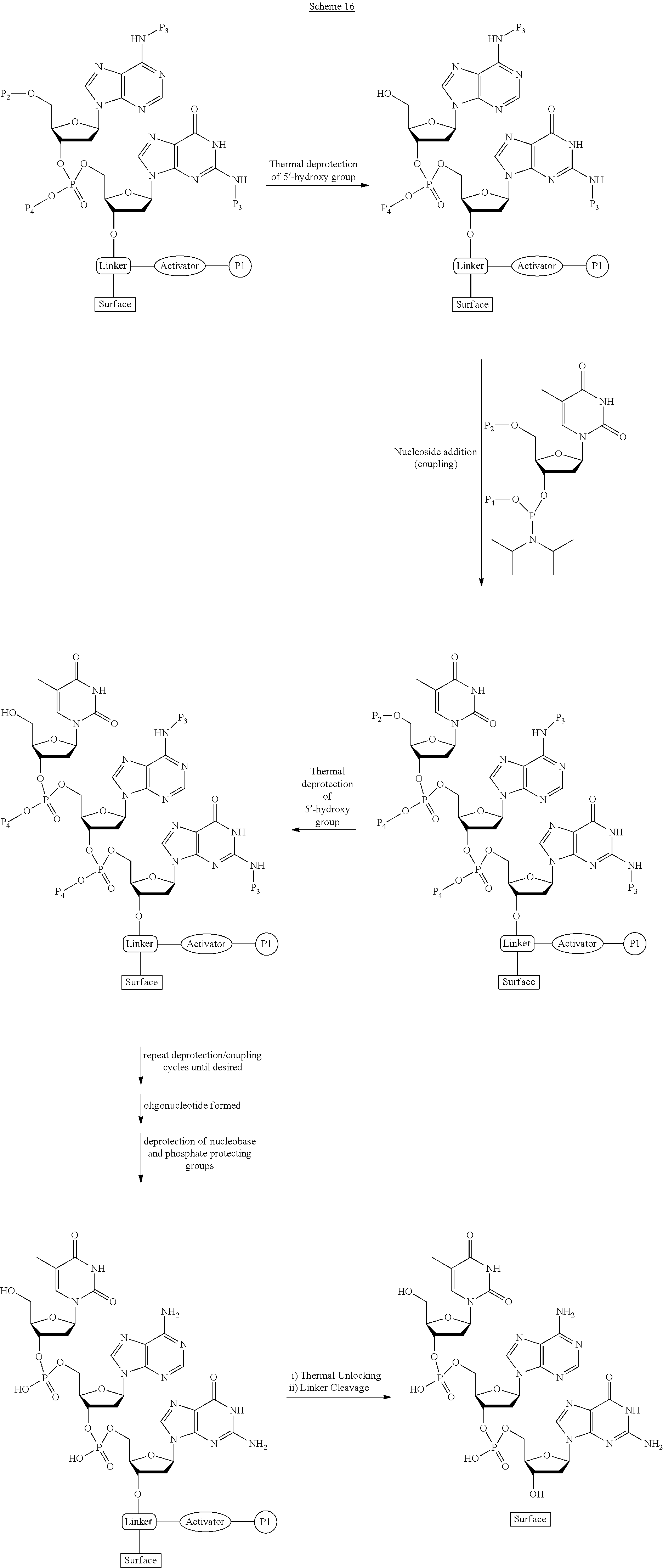

The following reaction scheme (Scheme 17) illustrates a process for the preparation of a nucleoside phosphoramidite reagent from nucleosides protected at the 5′ position with a thermally cleavable protecting group. The resulting nucleoside phosphoramidite reagent may be used, for example, in Scheme 16.

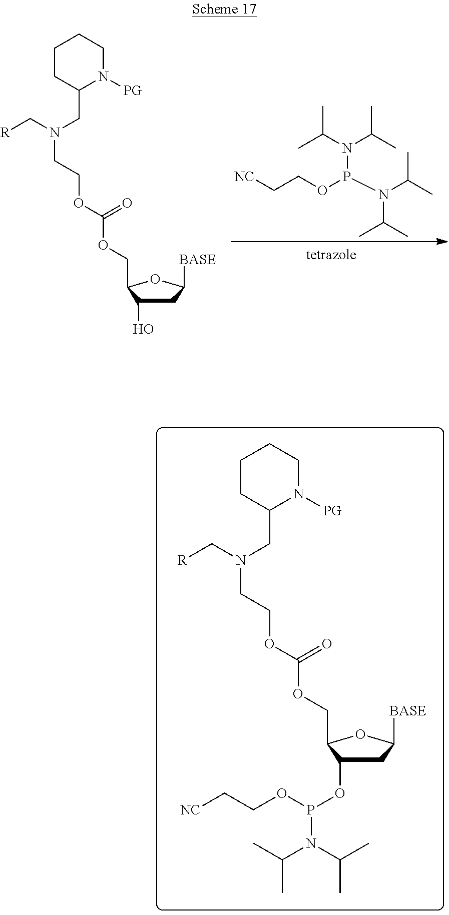

### Thermal Control

The present invention therefore enables the synthesis of nucleic acids from a plurality of individual oligonucleotide fragments. In order to achieve this, the process can comprise two separate thermally controlled chemical processes. Firstly, each starting nucleoside or nucleotide for the required oligonucleotide fragments is attached to the surface of the reaction sites via a thermally cleavable linker [step (i)]. Secondly, the individual oligonucleotide fragments that make up the desired polynucleotide, are synthesised with thermal control (steps (ii) and (iii)—thermally controlled 5′-OH deprotection and coupling at the 5′-OH deprotected sites). In any aspect of the invention, this phase may be carried out for the parallel synthesis of multiple polynucleotides. A third thermally controlled process comprises, on completion of the oligonucleotide fragment syntheses, thermally controlled release of the completed oligonucleotide fragments, which enables sequential transport, purification and ligation, and hence synthesis of the desired nucleic acids.

To enable chemical differentiation between reaction sites on the surface of the substrate, which can all be exposed to the same chemical reagents throughout the process, a hot or cold temperature can be applied at each site to control whether a reaction occurs or not. At the end of the synthesis, the cleavable linker together with the substrate can be quickly and selectively removed in order to isolate the synthesised organic compound.

Due to the highly degree of thermal control which enables highly selective 5′-OH protecting group removal, and highly controlled coupling at only the desired sites, the process of the present invention enables highly accurate synthesis of oligonucleotide sequences. Moreover, deletion and coupling errors are minimized. The process of the present invention can therefore be conducted without having the usual “capping” steps as employed in a standard phosphoramidite synthesis to remove e.g. deletion errors. Removal of the capping step also reduces the exposure of the oligonucleotides to further reagents, as well as improving the overall time and efficiency of the process.

### Temperature Control Device

In order to achieve the above-mentioned thermal control of the oligonucleotide synthesis and release, the substrate may comprise individually thermally addressable sites on a chip, thereby providing a temperature control device for controlling temperatures at a plurality of sites of the substrate comprising:
a plurality of active thermal sites disposed at respective locations on a substrate, each active thermal site comprising a heating element configured to apply a variable amount of heat to a corresponding site of said medium and a thermal insulation layer disposed between the heating element and the substrate; and
one or more passive thermal regions disposed between the plurality of active thermal sites on the substrate, each passive thermal region comprising a thermal conduction layer configured to conduct heat from a corresponding portion of the medium to the substrate;
wherein the thermal conduction layer of said one or more passive thermal regions has a lower thermal resistance in a direction perpendicular to a plane of the substrate than the thermal insulation layer of said plurality of active thermal sites.

The method for controlling temperatures at a plurality of sites of the substrate, can comprise:
providing the medium on a temperature control device comprising a plurality of active thermal sites disposed at respective locations on a substrate and one or more passive thermal regions disposed between the plurality of active thermal sites on the substrate;
   each active thermal site comprising a heating element configured to apply a variable amount of heat to a corresponding site of said medium and a thermal insulation layer disposed between the heating element and the substrate;
   each passive thermal region comprising a thermal conduction layer configured to conduct heat from a corresponding portion of the medium to the substrate; and
   the thermal conduction layer of said one or more passive thermal regions having a lower thermal resistance in a direction perpendicular to a plane of the substrate than the thermal insulation layer of said plurality of active thermal sites; and
controlling the amount of heat applied by the heating elements of the plurality of active thermal sites to control the temperatures at said plurality of sites of the medium.

In any aspect or embodiment of the present invention, the temperature control device can be manufactured by a process comprising:
forming a plurality of active thermal sites at respective locations on the substrate and one or more passive thermal regions disposed between the plurality of active thermal sites on the substrate; wherein:
each active thermal site comprises a heating element configured to apply a variable amount of heat to a corresponding site of said medium and a thermal insulation layer disposed between the heating element and the substrate;
each passive thermal region comprises a thermal conduction layer configured to conduct heat from a corresponding portion of the medium to the substrate; and
the thermal conduction layer of said one or more passive thermal regions has a lower thermal resistance in a direction perpendicular to a plane of the substrate than the thermal insulation layer of said plurality of active thermal sites.

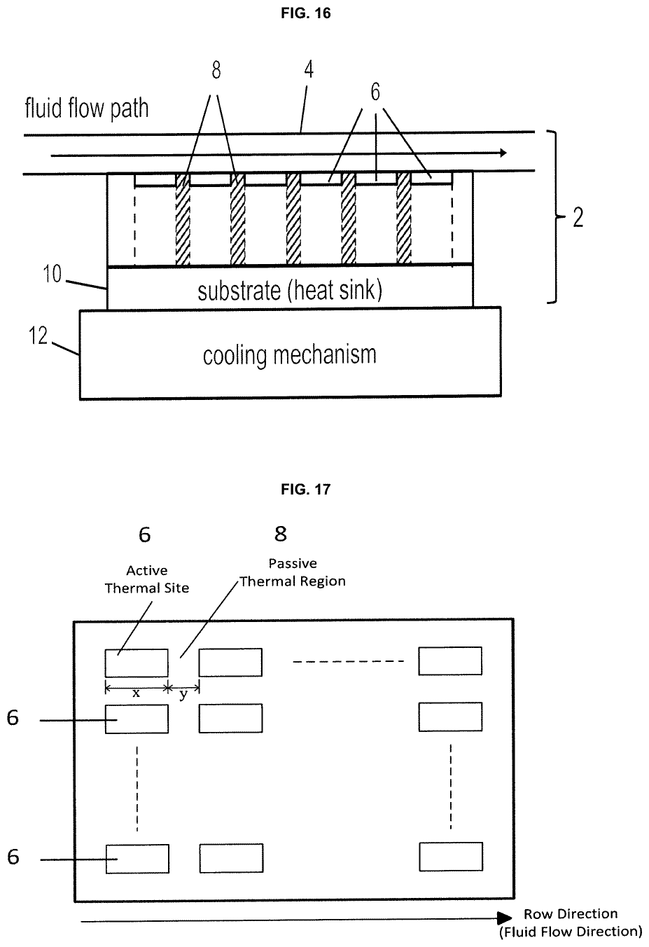

FIGS. 16-30 describe the temperature control device in more detail.

A temperature control device for controlling temperatures at multiple sites of a medium comprises a number of active thermal sites disposed at respective locations on a substrate, with each active thermal site comprising a heating element for applying a variable amount of heat to a corresponding site of the medium, and a thermal insulation layer disposed between the heating element and the substrate. One or more passive thermal regions are disposed between the active thermal sites on the substrate, with each passive thermal region comprising a thermal conduction layer for conducting heat from a corresponding portion of the medium to the substrate. The thermal conduction layer of the passive cooling region(s) has a lower thermal resistance in a direction perpendicular to a plane of the substrate than the insulating layer of the active thermal sites. When in use, the substrate can act as a heat sink (either by having the substrate exposed to room temperature, or by providing cooling of the substrate if lower temperatures are required). Hence, the thermal conduction layer in the passive regions enables the passive regions to provide cooling of the medium in the regions between the active thermal sites, so that less cooling needs to be provided by the active thermal sites themselves. This enables the active thermal sites to be designed to be more efficient for heating, since a thermal insulation layer which has a higher thermal resistance can be used between the heating element and the substrate as it is no longer required to allow so much heat to pass to the substrate to support cooling. This means that during heating less heat is lost to the substrate and therefore the overall temperature range supported by the device can be higher.

This can be contrasted with an alternative approach, which would be to provide a number of active sites which are the sole source of heating/cooling, each site having a heater with variable heat output, with cooling being provided when the heat from the heater is less than the heat lost to the substrate acting as a heat sink (with the boundaries between the active sites having the same or higher thermal resistance than the active sites). However, a problem with this approach is that when the medium above a given active site is at a relatively low temperature but further cooling is still required, the heat flow from the active site to the substrate would be relatively low (since the heat flow depends on the temperature difference across the heat flow path), and so to achieve further cooling the material of the active site would need sufficiently low thermal resistance that there is enough heat flow to the substrate at low temperatures. On the other hand, when the temperature at the corresponding site on the medium is relatively high, then the temperature difference across the heating site would be much larger and so the amount of heat lost to the substrate would be large. Therefore, to heat the corresponding site of the medium to even higher temperature, this would require a great amount of power to be applied to the heating element to counteract the heat lost to the substrate below. In practice, the maximum power supported by the heating element may be limited due to design constraints. Hence, an approach which uses the same site to provide the full heating/cooling functionality will be limited in the range of temperatures which can be controlled at a given site of the medium.

In contrast, with the present technique, the passive thermal regions between the active thermal sites include a layer which is more thermally conductive than the thermal insulation layer between the heating element and the substrate in the active sites. As cooling can be provided by the passive thermal regions, this means that the active sites do not need to provide as much cooling, and so can be made from a more thermally insulating material, so that less heat is lost to the substrate at the active sites and so more of the power of the heating element can be used for heating the medium itself. Hence, for a given amount of cooling to be provided and a given power available from the heating element, the maximum temperature achievable can be increased compared to the alternative approach discussed above. Hence, a wider range of temperatures can be controlled at each site using the temperature control device.

The passive sites are passive in the sense that, while the amount of cooling provided at the passive sites will depend on the temperature difference across them (which may indirectly depend on the temperature settings at neighbouring active sites), the temperature control device does not directly control the amount of heat flow at the passive sites, and instead the thermal conduction layer simply provides a given amount of thermal resistance to heat flow, which is a lower resistance than the thermal insulation layer at the active sites. As well as helping improve the range of temperatures achievable using the active sites, when the device is used for controlling temperatures within a flowing fluid, the passive regions can also help to reduce the “history” effect of heating at previous sites passed by the fluid, as the passive regions can cool the fluid closer to the substrate temperature to reduce the variability of the temperature of the fluid entering a given active site. This reduces the necessary loop gain of the control loop for controlling the heater at each site (see further discussion below).

On the other hand, the active sites are active in the sense that the amount of heating or cooling provided can be controlled by varying the power provided by the heating element. Nevertheless, the amount of heat flow to or from the medium at the active sites depends not only on the amount of heat provided by the heating element, but also on the temperatures around the active site, which may affect how much of the heat from the heating element is lost to the substrate or to surrounding passive thermal sites.

Hence, control circuitry may be provided to control whether a selected active thermal site provides heating of a corresponding site of the medium using the heating element or cooling of the corresponding site by heat flow through the thermal insulation layer to the substrate, in dependence on whether an amount of heat generated by the heating element in that active thermal site is greater or smaller than a threshold amount. The threshold amount may effectively represent the amount of heat that has to be generated by the heating element in order to counteract the heat lost to the substrate or the surrounding passive thermal sites.

This threshold amount may depend on a number of factors, including the thermal resistance of the thermal insulation layer of the active thermal site in a direction perpendicular to the plane of the substrate. For a given maximum heater power, the range of temperatures supported will tend to be shifted towards lower temperatures if the thermal insulation layer has a lower thermal resistance than if it has a higher thermal resistance. Hence, the bias point, at which the heating element counteracts the heat lost to surrounding areas other than the medium heat sink, can be carefully controlled by selecting an insulation layer with a given thermal resistance. Hence, different embodiments may be designed for different applications (depending on the required temperature ranges) by choosing insulation materials with different thermal resistance (e.g. by choosing different materials themselves, or by varying the physical structure of a given insulation material, such as by including voids).

The threshold amount may also be temperature dependent, e.g. a hotter active site will tend to lose more heat to the substrate than a cooler active site as there is a larger temperature difference across it. Hence, depending on the temperature of an active site, different amounts of the power may need to be delivered by the heating element in order to achieve a given amount of heat flow to the medium. This makes control of the heating element in order to provide a given temperature setting more complex.

Hence, each active thermal site may comprise a temperature sensor for sensing a temperature at the corresponding active thermal site. A number of feedback loops may be provided, each corresponding to one of the active thermal sites for controlling the heating element of that active thermal site. Each feedback loop may implement a transfer function for determining a target amount of heat to be applied to the corresponding site of the medium in dependence on the temperature sensed by the temperature sensor of the corresponding active thermal site and a target temperature specified for the corresponding site of the medium. A further function (referred to as a lineariser function below) may then map the target amount of heat determined by the transfer function to an input signal for controlling the heating element of the corresponding active thermal site. The lineariser function may be a function of the temperature sensed by the temperature sensor of the corresponding active thermal site, and may determine the input signal in dependence on a sum of the target amount of heat and an amount of heat lost from the heating element of the active thermal site to the substrate and surrounding passive thermal regions.

One might expect that a feedback loop for controlling the heater in dependence on the measured temperature at an active site should simply implement a single transfer function mapping the error between the target and measured temperatures directly to the heater input signal. However, such a control loop would be extremely challenging to implement in practice. Not all the heat supplied by the heater is supplied to the medium itself, as some heat is lost to the substrate via the thermal insulation layer in the active thermal sites or to passive thermal regions surrounding the active thermal site. The amount of heat lost to surrounding areas is temperature-dependent, and as each site can be at a different temperature, the heat lost varies from site to site. Hence, in a transfer function for which the plant is the heat provided by the heater, rather than the heat flow into the medium, the loop gain would become a function of the active site temperature, and so no unique controller (transfer function) would exist to ensure stability and accuracy over all possible active site temperatures.

In contrast, by separating the control of the heater into two parts, a stable control loop can be designed. The first part of control is the transfer function mapping the error between the measured and target temperatures to the target amount of heat to be supplied to the fluid (without considering how to control the heater to provide that target amount of heat). By providing a closed-loop control transfer function where the plant is the target amount of heat to be supplied to the medium rather than the amount of heat to be supplied by the heater, the loop gain can be made independent of the temperature of the site, which allows the loop to be modelled as a linear time invariant system according to classical control theory. On the other hand, the subsequent lineariser function maps the target amount of heat determined by the transfer function to the heater control input. The lineariser function can be designed according to a model of the heat flow at a given active site (dependent on the measured temperature of the active site). By bringing the temperature-dependent heat loss out of the closed-loop transfer function, the loop gain can effectively be “linearised” (approximated to a linear time invariant system), hence the term “lineariser function”. This allows for design of a stable control loop.

One may question why the closed-loop controller is provided, if one can already model the heat flow at an active site using the lineariser function—could a heat flow model representing the relationship between the target temperature and the power to be supplied by the heater be used without a closed loop transfer function? However, the amount of heat required to be supplied to the medium to set a given target temperature depends not only on the target temperature, but also on the previous temperature of the medium to be heated (there is some “history” to be accounted for). For heating of a solid medium, the history depends on previous temperature settings at a given active site (which could change over time). For heating of a fluid medium flowing over the active and passive sites, the history depends on the heating applied at other sites which the fluid passed before reaching the current active site. For example, if a given part of the fluid flows from a hotter site to a cooler site, we would expect to need to provide cooling to reduce the temperature rather than heating to increase the temperature, whereas the same target temperature setting for the second site could require heating if it follows an even cooler site. While the passive sites can help “reset” the temperature history by cooling the medium closer to the substrate temperature, there is still a history-dependent effect which would be difficult to account for with a simple heat flow model alone. By using a closed-loop approach where the target amount of heat to the fluid is continuously adjusted according to a certain transfer function dependent on the error between the target/measured temperatures, this enables us to achieve better temperature control (even if there is no actual knowledge of the previous temperature of the medium, e.g. the closed loop transfer function does not need to account for the actual temperature of the fluid arriving at an active site, which may still be unknown).

The relation used for the lineariser function can be derived as a function representing an analytic inversion of a thermal model of the temperature control device as will be described in more detail in the examples below. The thermal model may be a model in which thermal properties of heat flow, thermal resistance and thermal mass may be represented by electrical current, electrical resistance and electrical capacitance respectively, to allow the required non-linear control function to be derived by analogy to an electrical circuit.

In particular, the lineariser function may map the target amount of heat q_{fi} to an actual amount of heat q to be supplied by the heating element of a given active thermal site according to the following relation:

$$q = {q}_{fi} + \frac{{T}_{i} - {T}_{HS}}{\frac{{R}_{iz}}{2}} + \{ \frac{{T}_{i} - {R}_{1} ‖ {R}_{3} [ \frac{{T}_{i}}{{R}_{1}} + \frac{{T}_{HS}}{{R}_{3}} ]}{{R}_{1}} \}$$

where:
q_{fi} represents the target amount of heat to be supplied to the medium at the given active thermal site (determined as a function of the difference between the target temperature for the given active thermal site and the temperature sensed by the temperature sensor of the given active thermal site);
Tᵢ represents the temperature sensed by the temperature sensor of the given active thermal site;
T_{HS} represents the temperature of the substrate (acting as a heat sink);
R_{iz} represents the thermal resistance of the thermal insulation layer of the active thermal site in the direction perpendicular to the plane of the substrate;

$$\frac{1}{{R}_{1} ‖ {R}_{3}} = \frac{1}{{R}_{1}} + \frac{1}{{R}_{3}}$$

$${R}_{1} = \frac{1}{\frac{4}{{R}_{ix}} + \frac{4}{{R}_{iy}}} + \frac{1}{\frac{4}{{R}_{cx}} + \frac{4}{{R}_{cy}}}$$

$${R}_{3} = \frac{{R}_{cz}}{8}$$

Rᵢₓ and R_{iy} represent the thermal resistance of the thermal insulation layer of an active thermal site in two orthogonal directions parallel to the plane of the substrate;
R_{cx} and R_{cy} represent the thermal resistance of the thermal conduction layer of a passive thermal region in two orthogonal directions parallel to the plane of the substrate; and
R_{cz} represents the thermal resistance of the thermal conduction layer of a passive thermal region in the direction perpendicular to the plane of the substrate.

In some examples the heating element may comprise a resistive heating element. Although thermo-electric devices or other types of heating could also be used, a resistive heating element can be simpler to manufacture and control. For a resistive heating element, the current I to be applied to the heating element may be determined according to

$$I = \sqrt{\frac{q}{r}} ,$$

where q is determined according to the lineariser function as defined above and r is the resistance of the heating element.

In some examples, the thermal insulation layer in the active thermal sites may have a greater thermal resistance in a direction parallel to the plane of the substrate than in a direction perpendicular to the plane of the substrate. Making the thermal insulation layer less “leaky” in the lateral direction than across the thickness of the substrate allows the thermal insulation layer to support a given amount of cooling at the active thermal sites by heat flow to the substrate, while reducing the amount of heat lost through parasitic paths via surrounding passive thermal regions. Reducing the amount of heat lost to the passive regions not only makes heating at the active elements more efficient (a heater supporting a given maximum power can therefore support higher temperatures of the medium), but also simplifies the thermal model for deriving the non-linear control function discussed above, so that a simpler equation can be used which is less complex to implement in mapping circuitry. There are a number of ways in which the thermal insulation layer can be constructed to have a greater thermal resistance in the direction running in the plane of the substrate than in the transverse direction.

For example, the insulating layer could have a thin film structure, where the thickness z of the thermal insulation layer in the direction perpendicular to the plane of the substrate is substantially smaller than a smallest dimension L of the thermal insulation layer of the active thermal site in a direction parallel to the plane of the substrate. For example, z/L could be less than 0.1. In practice, z/L could be made smaller than 0.1, e.g. <0.05, or <0.01. In general, if the thickness is small in comparison to the lateral dimensions, then the thermal insulation layer will present a relatively large area for heat flow to the substrate, but a much smaller area for heat flow to the surrounding passive thermal regions, to provide more efficient heating and a simpler non-linear control function. The thin film approach can be suitable for some types of insulating material.

However, other types of insulating material may not have enough thermal resistance to provide sufficient insulation in the direction perpendicular to the plane if the thickness is reduced. For example, if silicon dioxide is used as the insulator, its inherent thermal conductivity may limit how thin the layer can be made if the thermal insulation layer is to provide enough insulation. While other materials could be chosen, silicon dioxide can be simpler to manufacture as it can allow the insulator to be formed by oxidisation of silicon used as a substrate for other parts of the device. Similarly, there may also be other materials for which the thin film approach (made of a single solid material) may not be practical given the required thermal insulation properties.

This can be addressed by providing a thermal insulation layer which comprises at least one void. The voids can be holes or pockets of air, another gas, or vacuum within the body of the temperature control device. As the thermal conductivity of air or vacuum can be relatively high compared to solid insulator materials, providing some voids can allow the thermal resistance in the in-plane and cross-plane directions to be controlled more carefully than is possible in a layer of a solid material.

In one example, the voids can extend substantially perpendicular to the substrate, with other portions of the thermal insulation layer made from a solid insulator material. For example, the thermal insulation layer may comprise one or more pillars of a first thermal insulation material extending substantially perpendicular to the plane of the substrate in the area of the active thermal site between the heating element and the substrate, and the voids may be disposed between or around the pillars. The voids and pillars may have a wide variety of shapes, and could pass through the entire thickness of the insulating layer, or only partially through part of the thickness. By providing voids and pillars which extend substantially perpendicular to the plane of the substrate, this can allow relatively efficient heat transfer in the direction perpendicular to the plane of the substrate (since heat can pass more easily through the more conductive pillars), but it can be more difficult for heat to flow laterally towards the passive cooling regions, because lateral heat flow would require crossing of one or more voids of air, gas or vacuum. The fill ratio (fraction of the total area taken up by the pillars or voids) can be varied to provide different ratios between the in-plane and cross-plane thermal resistance, to give precise control over the bias point for heating/cooling.

On the other hand, other examples may provide a thermal insulation layer which comprises a void extending substantially throughout the entire area of the active thermal site between the heating element and the substrate. Hence there may not be any need for any pillars. The thermal insulation layer could essentially comprise a layer made entirely of gas or vacuum (other than some solid bounds at the edge of the active thermal site).

Manufacturing of the device including a layer with voids can be achieved by forming the one or more voids within a device layer provided at a first surface of a primary wafer, and bonding the first surface of the primary wafer to a secondary wafer for supporting other elements of the thermal control device such as the heating element of each active thermal site and at least part of the thermal conduction layer of each passive thermal region. The voids could be formed either before or after the bonding of the primary and secondary wafers. Hence, by bonding primary and secondary wafers, it is possible to form voids within the body of the temperature control device.

However, where the thermal insulation layer comprises pillars and voids, the pillars can be formed in the device layer of the primary wafer prior to bonding it with the secondary wafer, and after bonding the primary wafer and the secondary wafer, the voids can be formed by etching away portions of the device layer between the pillars from an opposite side of the device layer to the first surface. For example, the first thermal insulation material may comprise an oxide (e.g. silicon dioxide), and the pillars may be formed in the device layer by etching holes in the device layer and oxidising material of the device layer at the edges of the holes to define the walls of the pillars. The primary wafer may comprise a buried oxide layer at an opposite side of the device layer from the first surface, and after bonding the primary wafer and the secondary wafer, the primary wafer can be etched back to the buried oxide layer, holes can be etched in the buried oxide layer at locations of the voids, and then parts of the device layer can be etched away via the holes in the buried oxide layer to form the voids. The holes in the buried oxide layer can then be filled by depositing more oxide to cover the holes. This approach allows the pillared structure to be manufactured using available silicon CMOS and silicon MEMS industrial processes. With this approach, the thickness of the device layer between the first surface and the buried oxide layer of the primary wafer will determine the height of the pillars in the thermal insulation layer, and the size of the holes etched into the primary wafer determines the size of the pillars and hence the fill ratio of pillars to voids. The size of the etch holes can be varied using a mask, allowing careful control over the ratio between the thermal resistances in the directions perpendicular and parallel to the plane of the substrate.

The temperature control device may comprise a cooling mechanism to cool the substrate to act as a heat sink. Alternatively the temperature control device may be provided without a cooling mechanism, and an external cooling mechanism can be used (e.g. the temperature control device can be placed with the substrate in contact with a cooling device to maintain the substrate at a given temperature), or the substrate could simply be held at room temperature. In general, the temperature of the substrate limits the lowest temperature that can be controlled at the active thermal sites, so depending on the particular application different amounts of cooling may be required.

While the temperature control device can be used to heat sites in a solid surface (e.g. for semiconductor temperature control) or in a static fluid, it is particularly useful for controlling the temperature at various sites within a flowing fluid. Hence, the temperature control device may comprise a fluid flow control element for controlling flow of the fluid over the plurality of active thermal sites and the one or more passive thermal regions. For example, for supporting chemical reactions, the flow of fluid may supply reagents for the reactions, and as the reagent flows over the various active thermal sites and passive thermal regions, it can be heated or cooled to desired temperatures suited to the reactions at each site. For example, the temperature can be used to control whether a reaction at a given site is triggered.

In one example, the active thermal sites may be disposed in one or more rows oriented substantially parallel to the direction of fluid flow controlled by the fluid flow control element. Each row may comprise two or more active thermal sites with a passive cooling region disposed between each pair of adjacent active thermal sites of the row. Disposing the sites in rows can make manufacture of the device more straightforward. In particular, if there are two or more rows, the active thermal sites can be arranged in a matrix structure, which can simplify addressing of individual sites for routing control signals to each site and reading out the temperatures measured at each site (e.g. a row/column addressing scheme can be used).

Hence, when fluid flows across the temperature control device, a given part of the fluid will flow along one of the rows which are oriented parallel to the fluid flow direction. That part of the fluid will encounter a given active thermal site, where it is heated or cooled to a given temperature, then flow over a passive site where it is subject to cooling, then encounter another active thermal site where it can be heated or cooled to a different temperature to the first active thermal site, and so on as it passes along the row. Each active thermal site may have a length along the row direction that is greater than a length along the row direction of each passive cooling region disposed between adjacent active thermal sites of the row. Making the active thermal sites longer than the intervening passive regions allows for more efficient use of the total area of the substrate (and hence greater number of control sites per unit area), as for the active thermal sites once the fluid has been brought to the desired temperature the fluid should remain at that temperature for some time to enable the reactions to take place, but when the fluid passes over the passive sites the only function is cooling (not supporting reactions), and so provided there is enough gap between active sites to provide sufficient cooling before the fluid reaches the next active site, there is no need for the temperature to remain constant within part of the passive regions. Hence, by making the passive regions smaller than the active regions, more reaction sites can be fitted within a given amount of space.

In some embodiments each active thermal site may include a reaction surface at the surface in contact with the medium. For example, the reaction surface could be made of gold, which can provide a neutral platform for many chemical or biological reactions.

A method for precisely controlling the temperature within spatially localised regions, ‘virtual wells’, of an extended body of flowing or static fluid is described. We achieve temperature control by a combination of passive cooling and resistive heating, allowing fast bidirectional control of the temperature within the virtual wells. In order to efficiently control the temperature and allow a wide range of liquid temperature, we both engineer the heat flow within the heater substrate chip and also develop a heat flow model that enables feedback control of the temperature.

For many chemical or biological processes, it can be useful to control chemical reactions at specific locations within a fluid. The rate at which chemical reactions occur is exponentially sensitive to temperature, enabling the ability to thermally control reaction rates. In order to achieve spatial control of thermally controlled chemical reactions, we describe a two-dimensional matrix of thermal sites (see FIGS. 16 and 17). To achieve bidirectional control over the temperature within the fluid, it is required both to pump heat into and out of the fluid. Here, we implement this bidirectional control of heat by using two species of thermal site, one whose primary purpose is to transfer heat into the fluid and the other whose primary purpose is to transfer heat out of the fluid.

FIG. 16 shows an example of a temperature control device 2 for controlling temperatures at respective sites in the medium. A fluid flow element (e.g. a pump) is provided to control the flow of fluid through a fluid flow path 4 across the top of the temperature control device 2. A number of active thermal sites 6 are provided at various locations across the plane of the temperature control device 2. The top of each active thermal site 6 may include a reaction surface (e.g. a gold cap) on which reactions can take place. Each active thermal site 6 includes a heating element to apply heat to the corresponding part of the fluid flowing over that site, to control the temperature of the fluid. As shown in FIG. 17, the active thermal sites 6 are arranged in a two-dimensional matrix (grid), arranged in two or more rows where the row direction is parallel to the direction that fluid flows through the fluid flow path 4. The regions lying between the active thermal sites 6 form one or more passive thermal regions 8 which do not comprise any heating element, but provide passive cooling by conducting heat away from the fluid towards the substrate 10 of the device 2. The length x of each thermal site 6 in the row direction is longer than the length y of each passive thermal region 8 lying between a pair of adjacent active thermal sites 6 in the same row. As shown in FIG. 16, a cooling mechanism 12 may be provided to cool the substrate 10 to act as a heat sink.

In principle, the same thermal site could both transfer heat into the fluid and transfer heat out of the fluid. For example, this could be achieved by a thermoelectric element, capable of bidirectional heat pumping. However, the approach described here defines two separate species of thermal site, which we refer to as active and passive sites 6, 8. A desirable attribute of the separated active and passive sites is that they can be fabricated by standard semiconductor processing techniques and by using materials available within that industry.

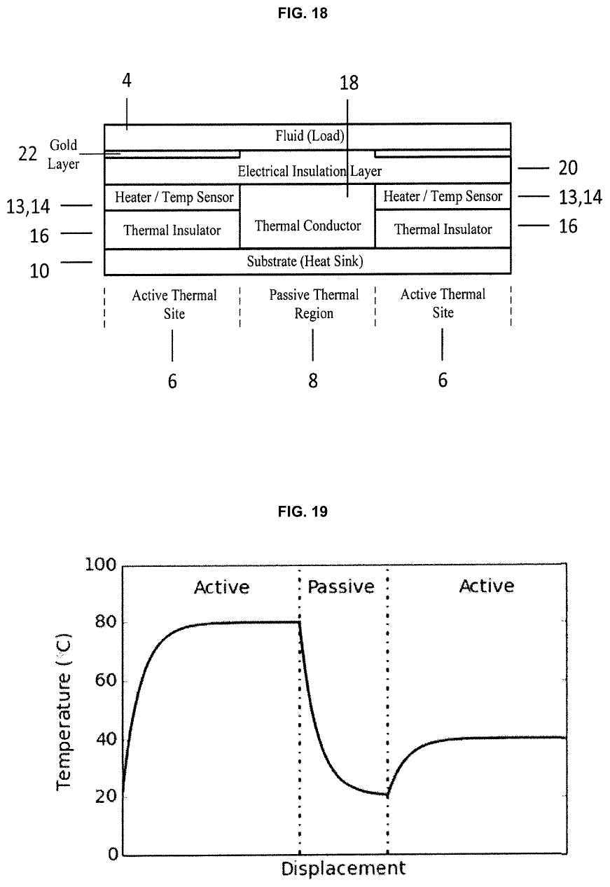

FIG. 18 shows a cross-section through the temperature control device 2 in more detail (FIG. 18 is schematic and is not intended to be to scale). The active thermal site 6 includes a heater 13 and thermometer (temperature sensor) 14. The heater 13 is operated under closed-loop control, with its output power set to maintain a certain temperature in the fluid above the site. The thermometer 14 in the active site provides the measurement for closed-loop control. Though the active site is primarily used to heat the fluid, at small heater powers, it is also capable of a small (relative to its ability to heat) amount of cooling, due to heat flow to the substrate 10. A thermal insulation layer 16 is provided between the heater 13 and the substrate 10 to control the amount of heat lost to the substrate 10. On top of the active site, the fluid is in contact with either an electrical insulator 20 or a gold pad 22, placed on the electrical insulator.

By contrast, the passive site 8 does not operate under closed-loop control and is responsible for transferring heat out of the fluid to a heat-sink at or underneath the substrate 10: the main role of the passive site is to act as a good thermal conductor. Hence, the passive region 8 includes a thermal conduction layer 18 for conducting heat from the fluid to the substrate 10. The temperature of the substrate 10 is maintained by a separate cooling mechanism 12 and can be assumed to be at a constant value. The passive sites are also covered by an electrically insulating region 20. The thermal conduction layer 18 of the passive site 8 has a lower thermal resistance in the direction perpendicular to the plane of the substrate than the thermal insulation layer 16 in the active site 6.

It will be appreciated that additional layers could also be included in the device 2 that are not shown in FIG. 18. For example, a heat spreading layer could be provided to spread heat from the heater 13 across the active thermal sites, to provide more uniform application of heat to the corresponding site.

As a fluid element moves over the surface of the chip 2, it passes, in an alternating fashion, over active and passive sites 6, 8. Over the active site, heat flows into the fluid and the temperature of the fluid element gets set to a desired ‘hot’ value. A short time later it passes over a passive site and the heat now flows out to the heat sink, leaving the fluid element at a ‘cold’ temperature. The fluid element then passes onto the next active site, and so on.

Hence, we include the passive thermal sites to pre-cool the fluid entering each active site, assuming that it is impractical for a resistive heater based active site to have commensurate cooling and heating ability. The passive sites 8 have the role of conducting heat away from the fluid, so that the fluid entering the space above an active site is close to the heatsink temperature. To illustrate the behaviour of the combined active and passive sites FIG. 19 shows a sketch of the temperature above an active-passive-active sequence. The leftmost active site pumps heat into the fluid, increasing its temperature to a maximum value of 80° C. Then as the fluid passes over the passive site it cools down towards 20° C. And finally as the fluid passes the rightmost active site heat flows in, and its temperature increases to 40° C. While these temperatures are arbitrary they are representative of operating conditions. As shown in FIG. 17, the active sites may have greater spatial extent than the passive sites (length x>length y). While the active sites provide a region of constant temperature for chemical reactions to take place, the only requirement of the passive sites is that they cool the fluid entering the active sites. This pre-cooling reduces the cooling requirement of the active sites, enabling them to more efficiently transfer heat into the fluid.

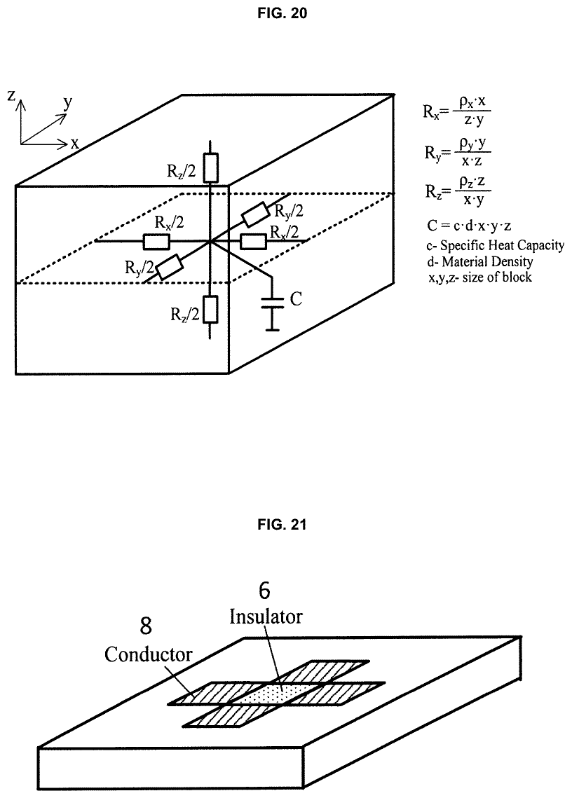

In order to design the thermal properties of the active and passive sites we describe the system by a thermal model. Here, we develop an electrical analogy in which the thermal resistances are replaced by electrical resistances; the heat capacity by capacitors; and temperature by voltage. To discretise the structure, and enable the construction of an electrical circuit, we divide it into blocks as shown in the thermal model of FIG. 20. A block may consist of an active or passive thermal site or a block of fluid above one of these sites.

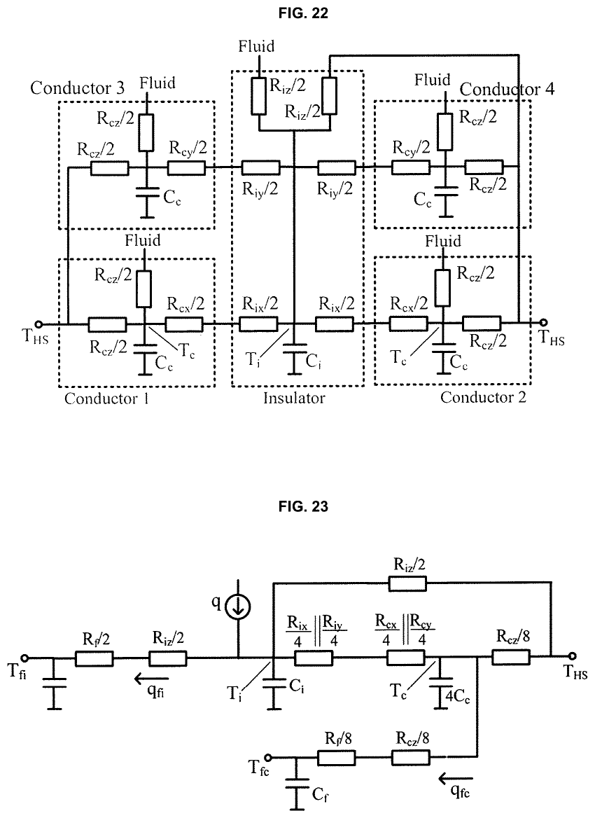

As a first order approximation of our system, we consider each active site to be surrounded by 4 passive sites (FIG. 21 showing insulator (6) and conductor (8)). By describing each active and passive site as a single thermal block, it is possible to draw a circuit diagram that describes an electrical model of the thermal behaviour of an active site (FIG. 22), where “conductor” or “conductive site” refers to the passive thermal region 8 and “insulator” or “insulating site” refers to the active thermal site 6, and:
C_{c} and Cᵢ—Heat capacity of the conductor and insulator respectively
R_{cx}, R_{cy}, R_{cz}—Thermal resistances of the conductor in x, y, z directions (where z is the direction perpendicular to the plane of the substrate 10 and x and y are orthogonal directions parallel to the plane of the substrate)
Rᵢₓ, R_{iy}, R_{iz}—Thermal resistances of the insulator in x, y, z directions
T_{HS}—Temperature of the heat-sink
T_{c} and Tᵢ—Temperature of conducting and insulating sites

Due to the symmetry of the physical structure and because of the isothermal substrate, we consider equal heat flow from the insulating region to the four conducting regions, enabling them to be considered together. In FIG. 23 we show a compacted thermal model, including this simplification, where we also include a heat flow or heat current (q) generated by the heater.
q—Heat current generated by the heater.
q_{fc}, q_{fi}—Heat current absorbed by the fluid through the conducting site and insulating site, respectively.
C_{f}— Thermal capacity of a block of fluid. It has a volume given by the area of the conducting (or insulating) site and height of the fluid, h_{f}.
R_{f}— Thermal resistance of a block of fluid. It has a volume given by the area of the conducting (or insulating) site and height of the fluid, h_{f}.
T_{fc}, T_{fi}—Temperature of the fluid above the conducting and insulating sites, respectively.

Using the electrical model of the thermal circuit, we can determine the heat flow from the insulating site into the fluid, q_{fi}. Taking the circuit in FIG. 23, we simplify the resistances as:

$${R}_{1} = \frac{{R}_{ix}}{4} ‖ \frac{{R}_{iy}}{4} + \frac{{R}_{cx}}{4} ‖ \frac{{R}_{cy}}{4}$$

$${R}_{2} = \frac{{R}_{cz}}{8} + \frac{{R}_{f}}{8}$$

$${R}_{3} = \frac{{R}_{cz}}{8}$$

where ∥ is shorthand for the equivalent combined resistance to the parallel resistances, e.g.

$$\frac{{R}_{ix}}{4} ‖ \frac{{R}_{iy}}{4} = \frac{1}{\frac{4}{{R}_{ix}} + \frac{4}{{R}_{iy}}}$$

Since the thermal current through R₁ is the sum of thermal currents into R₂ and R₃:

$$\frac{{T}_{i} - {T}_{c}}{{R}_{1}} = \frac{{T}_{c} - {T}_{fc}}{{R}_{2}} + \frac{{T}_{c} - {T}_{HS}}{{R}_{3}}$$

Therefore, we are able to write the thermal current (q_{fi}) passing through R₁ as:

$${q}_{1} = \frac{{T}_{i} - {T}_{c}}{{R}_{1}} = \frac{{T}_{i} - {R}_{1} ‖ {R}_{2} ‖ {R}_{3} [ \frac{{T}_{i}}{{R}_{1}} + \frac{{T}_{fc}}{{R}_{2}} + \frac{{T}_{HS}}{{R}_{3}} ]}{{R}_{1}} \\ {R}_{1} ‖ {R}_{2} ‖ {R}_{3} = \frac{1}{\frac{1}{{R}_{1}} + \frac{1}{{R}_{2}} + \frac{1}{{R}_{3}}}$$

We know the temperature Tᵢ, because we measure it with a temperature sensor, and we can calculate the heat flow from the insulator into the fluid, q_{fi}.

$${q}_{fi} = q - \frac{{T}_{i} - {T}_{HS}}{\frac{{R}_{iz}}{2}} - \{ \frac{{T}_{i} - {R}_{1} ‖ {R}_{2} ‖ {R}_{3} [ \frac{{T}_{i}}{{R}_{1}} + \frac{{T}_{fc}}{{R}_{2}} + \frac{{T}_{HS}}{{R}_{3}} ]}{{R}_{1}} \}$$

Due to the relatively low thermal conductivity of fluid (k_{f}=0.6 W/m/K) compared to silicon (k_{Si}=130 W/m/K) the thermal resistance of the conductor to the heat sink is much lower than the thermal resistance of the conductor to the fluid. Hence,

R₂>>R₃

With this assumption, the heat flow from the insulator into the fluid becomes:

$${q}_{fi} = q - \frac{{T}_{i} - {T}_{HS}}{\frac{{R}_{iz}}{2}} - \{ \frac{{T}_{i} - {R}_{1} ‖ {R}_{3} [ \frac{{T}_{i}}{{R}_{1}} + \frac{{T}_{HS}}{{R}_{3}} ]}{{R}_{1}} \}$$

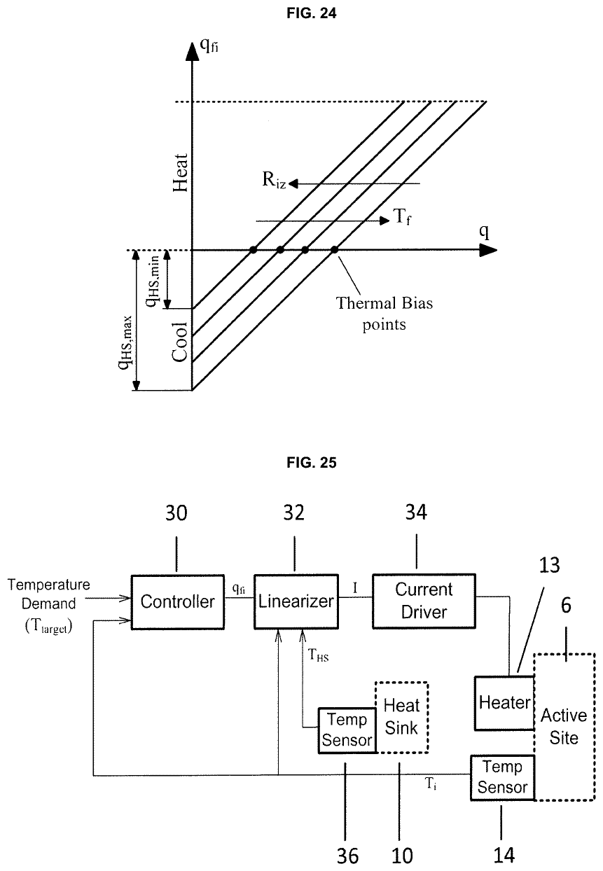

FIG. 24 plots heat flow into the fluid (q_{fi}), for several constant values of fluid temperature. In the case of zero heat output by the heater (assuming that T_{f}>T_{HS}), the heat flow from the insulator into the fluid (q_{fi}) is negative: i.e. the active site cools the fluid. The maximum amount of cooling provided by the active site is tuned by the thermal resistance R_{iz} of the thermal insulation layer 16 between the active site and the heat sink in the direction perpendicular to the plane of the substrate, and therefore that resistance R_{iz} is a key design parameter for the active site. As shown in FIG. 24 the bias point where the heat q from the heater exactly counteracts the loss of heat to the substrate 10 and surrounding passive regions 8 decreases with increasing insulator thermal resistance R_{iz}. Hence, the insulator resistance R_{iz} can be tuned to change the balance between heating and cooling at the active thermal sites 6.

The minimum available cooling power, which occurs when the heater is off and the temperature of the fluid is at a minimum, is set by the heatsink temperature and the thermal resistance of the site. However, unless the heatsink temperature is held at unrealistically low values, the amount of heat flowing through the site increases with the temperature of the fluid, i.e. q_{HS,max}>>q_{HS,min}. This inefficiency ultimately limits the cooling power that can be applied by the active site, because of the finite capacity of the heatsink to remove the waste heat. This is why providing the passive sites for pre-cooling the fluid between active sites enables more efficient heating and a larger temperature range for a given amount of heater power.

As discussed in the previous section, the thermofluidic chip described here has intrinsic non-linearity caused by the variable temperature of the fluid above the active sites. Hence we describe a thermal control system (see FIG. 25), which includes a non-linear control function (“lineariser”) in order to achieve the necessary temperature control. In this way, the electrical current passing through the heater 13 can be controlled in order to maintain a constant temperature in the fluid.

FIG. 25 shows the feedback loop for a single active site 6. Each active site 6 may have a separate instance of such a feedback loop. The target temperature T_{target} is input to a controller 30 which also receives the temperature Tᵢ measured by the temperature sensor 14 of the corresponding active site. The controller 30 determines the target amount of heat q_{fi} to be supplied by the active site 6 to the fluid based on a transfer function of the form C(s). (T_{target}−Tᵢ), where C(s) is a transfer function whose poles and zeros have been placed according to classical control theory.

A lineariser 32 comprises mapping circuitry which maps the target amount of heat q_{wi} supplied by the controller 30 to an input signal I defining the amount of current to be supplied by a current driver 34 to the heater 13, in dependence on Tᵢ and T_{HS}, the temperature of the substrate 10. The substrate temperature T_{HS} can be measured by a single sensor 36 shared between all active sites 6 or by individual sensors local to each active site 6. The lineariser 32 provides a non-linear mapping function which enables the controller 30 to use a linear transfer function (hence the term “lineariser”). The non-linear function provided by the lineariser 32 may be a function representing an analytic inversion of the thermal model. From the model described above, the total power generated into the heater to achieve the demanded temperature into the fluid is:

$$q ( {q}_{fi} , {T}_{i} , {T}_{HS} ) = {q}_{fi} + \frac{{T}_{i} - {T}_{HS}}{\frac{{R}_{iz}}{2}} + \{ \frac{{T}_{i} - {R}_{1} ‖ {R}_{3} [ \frac{{T}_{i}}{{R}_{1}} + \frac{{T}_{HS}}{{R}_{3}} ]}{{R}_{1}} \}$$

The electrical current necessary for the heater to reach a certain temperature is:

$$I = \sqrt{\frac{q}{r}} ,$$

where r is the electrical resistance of the heater.
Combining the two previous equations, we get the form of the lineariser, which converts heat demand into required current:

$$I = \sqrt{\frac{{q}_{wi} + \frac{{T}_{i} - {T}_{HS}}{\frac{{R}_{iz}}{2}} + \{ \frac{{T}_{i} - {R}_{1} ‖ {R}_{3} [ \frac{{T}_{i}}{{R}_{1}} + \frac{{T}_{HS}}{{R}_{3}} ]}{{R}_{1}} \}}{r}}$$

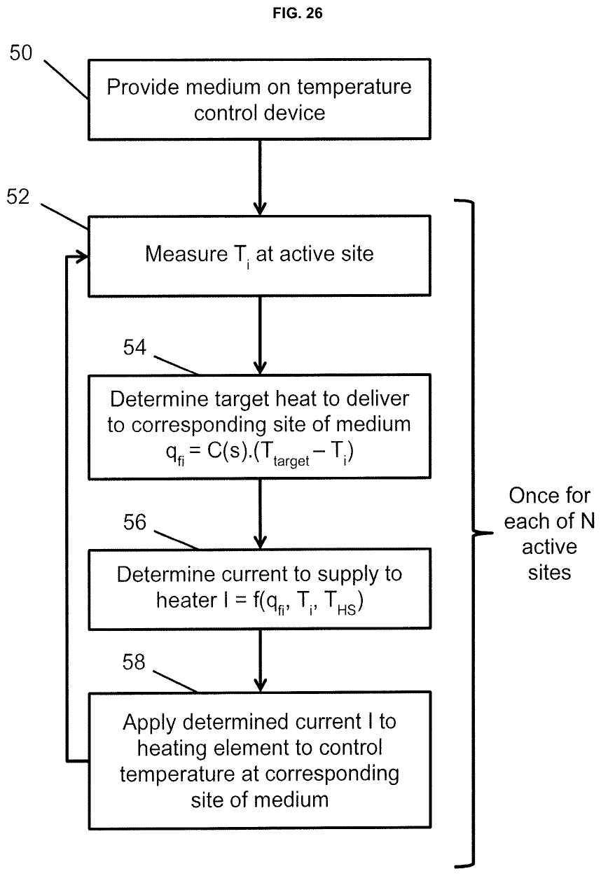

FIG. 26 is a flow diagram illustrating the temperature control method. At step 50 the medium in which the temperature is to be controlled is provided on the temperature control device. For example, the medium can be a fluid flowing over the temperature control device. At step 52, the temperature T, is measured at an active thermal site 6. At step 54 the target amount of heat to be delivered to the corresponding site of the medium is determined according to q_{fi}=C(s). (T_{target}−Tᵢ). At step 56 the current to be supplied to the resistive heater 13 is determined according to I=f(q_{fi}, Tᵢ, T_{HS}) where f is the function representing the lineariser equation shown above. At step 58 the determined amount of current I is supplied to the heating element 13 by the current driver 34 to control the temperature at the corresponding site of the medium. The method then returns to step 52 to continue to control the temperature at the site based on the measured temperature Tᵢ and target temperature T_{target}, taking into account the heat flow from the active site 6 to regions other than the medium itself according to the thermal model discussed above. Steps 52 to 58 are performed N times in parallel, once for each active site in the temperature control device 2.

To achieve temperature control of the active site, the required thermal resistances of the active and passive regions 6, 8 are determined, so that suitable materials and geometries can be chosen. There are two conditions which a 3D block of an active site should meet:

1—The power generated by the heater should mostly heat the fluid, and only a small fraction should leak vertically into the heat-sink i.e. the active site should have a high thermodynamic efficiency, η.

$$η = \frac{{q}_{fi}}{q}$$

2—The power generated by the heater should not flow horizontally towards other thermal sites i.e.

$${R}_{z} ≪ \frac{{R}_{x}}{4} ‖ \frac{{R}_{y}}{4}$$

This inequality can be satisfied either by using a thin film material for the thermal insulation layer 16 of an active site (such that z<<x,y, where z is the thickness in the direction perpendicular to the plane of the substrate and x, y are the in-plane length/width of the thermal insulation layer) or by use of an anisotropic thermal material, which is more thermally conductive in the direction through the thickness of the substrate than along the plane of the substrate (k_{z}>>kₓ,k_{y}).

We invoke this second requirement mainly to simplify a model for the heat flow, enabling a lineariser function to be simply determined. It would also be possible to design the active site for the other limit, in which there is no vertical transport of heat from the active site into the heat-sink. The reason that we consider the vertical transport limit is that it gives a better knowledge of the heat flow into the fluid. In the horizontal transport limit, there is an additional region of the chip's surface, with a temperature gradient, from which heat can flow into the fluid.

There are a number of materials out of which one could fabricate an active site but, as an example, let's consider SiO₂, a common material with a low thermal conductivity (k_{SiO2}=1.3 W/m/K). The thermal resistance for the active site material in the z-direction can be expressed as a function of the maximum heat leaking to the heat sink:

$${R}_{iz} = \frac{{T}_{i , max} - {T}_{HS}}{{q}_{HS , max}} = \frac{1}{{k}_{z}} \cdot \frac{z}{xy}$$

From this we can deduce the required height of material:

$$z = \frac{{xyk}_{z} ( {T}_{i , max} - {T}_{HS} )}{{q}_{HS , max}}$$

It remains to determine the maximum acceptable heat leakage to the heat sink, q_{HS,max}. For a rectangular active site of dimension 100 μm×200 μm, we assume a maximum heater power of 6 mW. At maximum heater power, we allow half of the power to go to the heatsink. Furthermore we assume a maximum fluid temperature of T_{f,max}=90 C, a heat sink temperature of T_{HS}=10 C and that the temperature of the thermal site is approximately the same as the temperature of the fluid (T_{f,max}≈T_{i,max}). If all the material of the active site is made from SiO₂, a material with isotropic thermal conductivity, then its height would need to be ≈700 μm. For such a block, the thermal resistance in the vertical direction is R_{iz}≈27,000 K/W. Such a block, for which z>x,y, does not satisfy the second condition of small heat leakage between thermal sites.

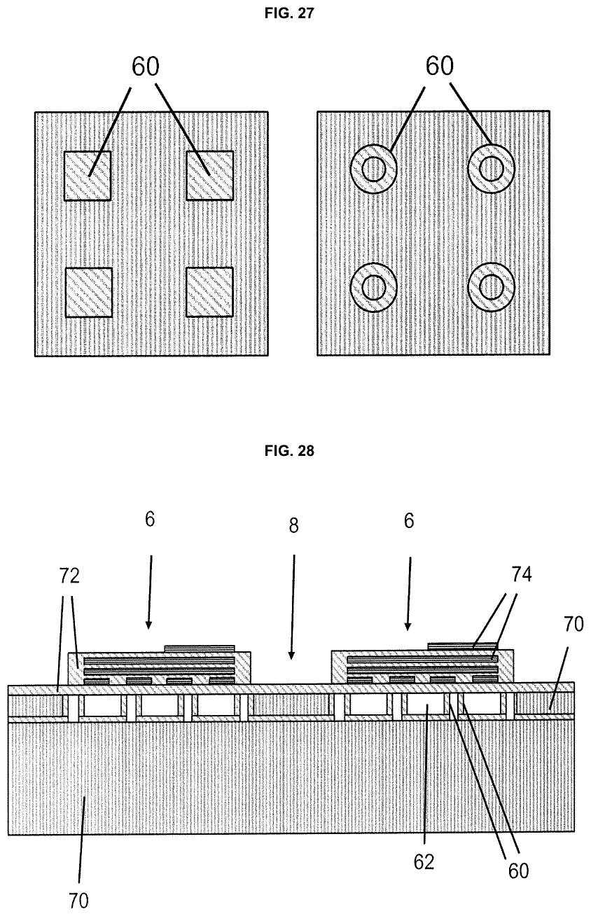

One way to satisfy the condition for small heat leakage between sites is to, by patterning, make the active site material thermally anisotropic. For example, one can produce a structure where vertical pillars of SiO₂ are separated by spaces of air (kₐᵢᵣ=0.024 W/m/K). The required vertical height of the material, in this case the height of the pillars, is multiplied by the pillar fill factor. For example, with a fill-factor of 10% the pillar height becomes 70 μm. The insulating pillars may take a number of different geometries, several examples of which are shown in FIG. 27. The pillars 60 are surrounded by voids comprising air, gas or vacuum. In other examples the pillars could enclose the voids.

By providing a pillared structure comprising pillars extending in the direction perpendicular to the substrate and voids around or between the pillars, we maintain the same thermal resistance in the vertical direction (R_{iz}≈27,000 K/W) but it is clear that the lateral resistance is reduced, mainly because kₐᵢᵣ<k_{SiO2} but also because of the lower height of the active material.

Calculating the lateral thermal resistances for a 10% fill-factor we find that:

$$\frac{{R}_{x}}{2} \approx \frac{y}{2 {k}_{air} xz} , \frac{{R}_{y}}{2} \approx \frac{x}{2 {k}_{air} yz}$$

This gives a total lateral thermal resistance of:

$$\frac{{R}_{x}}{4} ‖ \frac{{R}_{y}}{4} = 60 , 000 K / W$$

Note that the lateral thermal resistance can be further increased by reducing the pillar height, and simultaneously reducing the fill-factor. Alternatively, the silicon pillars can be separated by vacuum, providing a significant further increase in lateral resistance.

However, as the lateral thermal resistance in the bulk of the active material becomes large, it becomes important to consider the lateral thermal resistance of the capping layer. For example, a 2 μm thick silicon dioxide capping layer gives a contribution to the total lateral thermal resistance of:

$$\frac{{R}_{x}}{4} ‖ \frac{{R}_{y}}{4} = 38 , 000 K / W$$

In summary, patterning a thermal insulator to consist of insulating pillars separated by air (or vacuum) provides a method of satisfying the thermal conditions of an active site. The limit of this case (where fill-factor goes to zero and the void covers the entire area of the active site) results in a free standing membrane which can be considered as an alternative approach to satisfying the thermal requirements.

FIG. 28 shows how the pillared approach can be integrated into a complete device. The figure shows a cross-section through the device substrate, passing through two active and several passive thermal sites. Silicon 70 is shown using vertical hatching, silicon dioxide 72 using diagonal hatching, and the metal layers 74 are shown using horizontal hatching. The voids are shown in white. Note that the figure is not to scale, the upper layers are shown magnified in the vertical direction. Silicon provides a highly thermally conducting material for the substrate and is capable of being thermally oxidised in order to produce thermally insulating pillars 60 with voids 62 between the pillars. On top of the substrate containing the pillar structure there are a number of layers which contain the heater; a heat spreader (to evenly distribute the heat produced); a thermometer (to enable thermal control); and a surface capping layer.

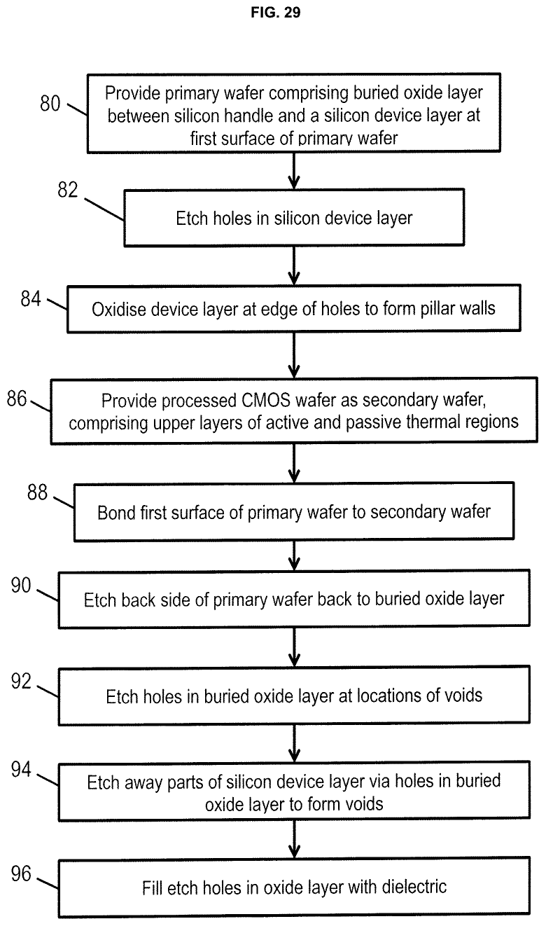

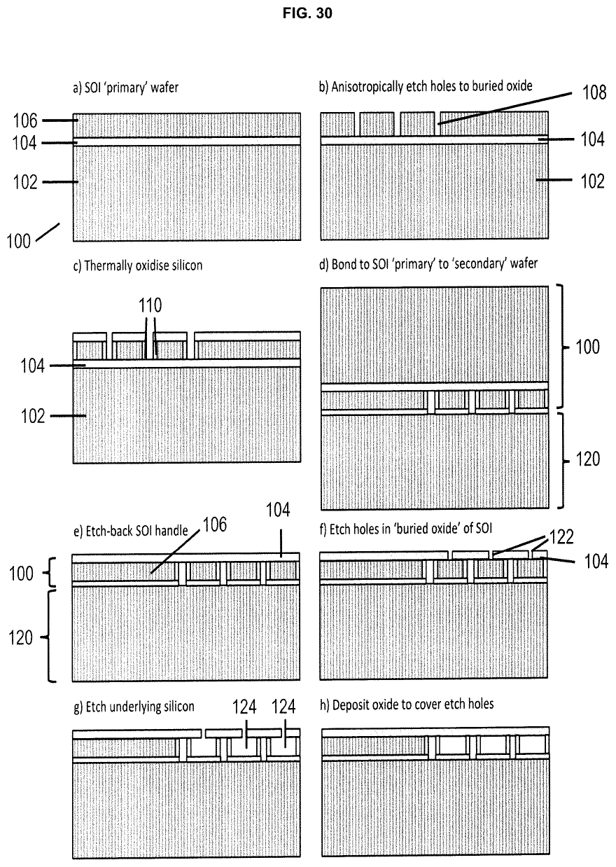

The device 2 of FIG. 28 can be built using processes available to the silicon CMOS and silicon MEMS industries. FIG. 29 shows a process flow which achieves the required thermal resistances in the passive and active regions and FIG. 30 shows the respective stages of the manufacturing method of FIG. 29. At step 80 of FIG. 29 (part a of FIG. 30), the process starts with a silicon-on-insulator (SOI) wafer 100 comprising a relatively thick silicon handle 102, a buried oxide layer 104, and a silicon device layer 106. The thickness of the silicon device layer 106 gives the height of the silicon dioxide pillars and the thickness of the buried oxide is approximately 1 μm. Since a second wafer is later used in the processing, we refer to the SOI wafer as the ‘primary’. The surface of the primary wafer 100 at which the device layer 106 is formed is referred to below as the ‘first surface’.

At step 82 (part b of FIG. 30), the primary wafer 100 is photolithographically patterned and, using photoresist as an etch mask, the silicon device layer 106 is anisotropically etched down to the buried oxide 104 to form holes 108. In order to achieve the etching anisotropy, a deep reactive ion etch is used.

At step 84 (part c of FIG. 30), the wafer is oxidised, giving a thermal oxide with thickness of approximately 1 μm for example. The edges of the holes 108 are oxidised to form the walls of the silicon dioxide pillars 110.

At step 86 a secondary wafer 120 is provided. The secondary wafer 120 comprises a processed CMOS wafer, containing the electrically active and electrically passive devices needed for the heating and control functionality (e.g. the heater 13, temperature sensor 14, and upper parts of the thermal conductor layer of the passive sites 8). These metal layers and the devices within the secondary CMOS wafer 120 are not shown in FIG. 30 but can be provided as shown in FIG. 28.

At step 88 (part d of FIG. 30), the primary wafer 100 is turned upside down and the first surface of the primary wafer 100 is bonded to the secondary wafer 120. The wafer bonding could be achieved by thermo-compression bonding, in which case metal (e.g. gold) layers are needed on the surface of both the primary and secondary wafers.

At step 90 (part e of FIG. 30), the backside of the bonded primary wafer (the original handle layer 102 of the SOI wafer) is etched back to leave the buried oxide 104 of that SOI wafer 100 as at the top of the stack. After this step, metal tracks for the heater/thermometer/heat-spreader stack can be built on the secondary wafer 120 (not shown in FIG. 30).

Since the voids in the silicon device layer from the original SOI wafer still need to be removed, at step 92 (part f of FIG. 30), etch holes 122 are photolithographically patterned and etched in the top silicon dioxide layer 104. Then, in a subsequent process step 94 (part g of FIG. 30), an anisotropic dry etch (e.g. with XeF₂) of these silicon regions is performed, to form the voids 124 by etching away parts of the silicon device layer 106 via the etch holes 122 in the oxide 104. At step 96, the etch holes 122 in the oxide layer 104 are filled with dielectric (part h of FIG. 30), completing the processing of the active and passive thermal sites.

In the present application, the words “configured to . . . ” are used to mean that an element of an apparatus has a configuration able to carry out the defined operation. In this context, a “configuration” means an arrangement or manner of interconnection of hardware or software. For example, the apparatus may have dedicated hardware which provides the defined operation, or a processor or other processing device may be programmed to perform the function. “Configured to” does not imply that the apparatus element needs to be changed in any way in order to provide the defined operation.

In a preferred aspect of the process of the present invention, the substrate is provided with a temperature control device for controlling temperatures at a plurality of sites of a medium, comprising:
a plurality of active thermal sites disposed at respective locations on a substrate, each active thermal site comprising a heating element configured to apply a variable amount of heat to a corresponding site of said medium and a thermal insulation layer disposed between the heating element and the substrate; and
one or more passive thermal regions disposed between the plurality of active thermal sites on the substrate, each passive thermal region comprising a thermal conduction layer configured to conduct heat from a corresponding portion of the medium to the substrate;
wherein the thermal conduction layer of said one or more passive thermal regions has a lower thermal resistance in a direction perpendicular to a plane of the substrate than the thermal insulation layer of said plurality of active thermal sites.

Preferably, the temperature control device comprises control circuitry configured to control whether a selected active thermal site provides heating of the corresponding site of the medium using the heating element or cooling of the corresponding site by heat flow through said thermal insulation layer to said substrate, in dependence on whether an amount of heat generated by the heating element of said selected active thermal site is greater or smaller than a threshold amount. Preferably, the threshold amount is dependent on the thermal resistance of the thermal insulation layer in the direction perpendicular to the plane of the substrate.

Preferably, in the temperature control device, each active thermal site comprises a temperature sensor configured to sense a temperature at the corresponding active thermal site. More preferably, the temperature control device comprises a plurality of feedback loops each corresponding to a respective active thermal site;
each feedback loop configured to implement a transfer function for determining a target amount of heat to be applied to the corresponding site of the medium in dependence on the temperature sensed by the temperature sensor of the corresponding active thermal site and a target temperature specified for the corresponding site of the medium.

Even more preferably, each feedback loop is configured to implement a lineariser function to map said target amount of heat determined by the transfer function to an input signal for controlling the heating element of the corresponding active thermal site. Preferably, the lineariser function is a function of the temperature sensed by the temperature sensor of the corresponding active thermal site. In preferred embodiments, the lineariser function determines the input signal in dependence on a sum of the target amount of heat and an amount of heat lost from the heating element of the active thermal site to the substrate and surrounding passive thermal regions.

The temperature control device preferably comprises a resistive heating element.

Preferably, the thermal insulation layer of said plurality of active thermal sites in the temperature control device has a greater thermal resistance in a direction parallel to the plane of the substrate than in a direction perpendicular to the plane of the substrate.

More preferably, the thermal insulation layer of a given active thermal site of the temperature control device comprises a thin film material having a thickness z in the direction perpendicular to the plane of the substrate which is substantially smaller than a smallest dimension L of the thermal insulation layer of the active thermal site in a direction parallel to the plane of the substrate.

The thermal insulation layer may comprise one or more voids. Preferably, the voids extend in a direction substantially perpendicular to the plane of the substrate.

The thermal insulation layer of the temperature control device may particularly comprise one or more pillars of a first thermal insulation material extending substantially perpendicular to the plane of the substrate in the area of the active thermal site between the heating element and the substrate, wherein said one or more voids are disposed between or around the pillars.

The thermal insulation layer can comprise a void extending throughout the entire area of the active thermal site between the heating element and the substrate.

The temperature control device may comprise cooling mechanism to cool the substrate to act as a heat sink.

Preferably, the medium comprises a fluid, and the temperature control device comprises a fluid flow control element configured to control flow of the fluid over the plurality of active thermal sites and the one or more passive thermal regions. Preferably, the active thermal sites are disposed in one or more rows oriented substantially parallel to the direction of fluid flow controlled by the fluid flow control element;
each row comprising two or more active thermal sites with a passive cooling region disposed between each pair of adjacent active thermal sites of the row. More particularly, each active thermal site has a length along the row direction that is greater than a length along the row direction of each passive cooling region disposed between adjacent active thermal sites of the row.

In use, the temperature control device can be used to control temperatures at a plurality of sites of the substrate, comprising:
providing the medium on a temperature control device comprising a plurality of active thermal sites disposed at respective locations on a substrate and one or more passive thermal regions disposed between the plurality of active thermal sites on the substrate;
   each active thermal site comprising a heating element configured to apply a variable amount of heat to a corresponding site of said medium and a thermal insulation layer disposed between the heating element and the substrate;
   each passive thermal region comprising a thermal conduction layer configured to conduct heat from a corresponding portion of the medium to the substrate; and
   the thermal conduction layer of said one or more passive thermal regions having a lower thermal resistance in a direction perpendicular to a plane of the substrate than the thermal insulation layer of said plurality of active thermal sites; and
controlling the amount of heat applied by the heating elements of the plurality of active thermal sites to control the temperatures at said plurality of sites of the medium.

The temperature control device can be manufactured by any suitable method. Preferably, the method comprises:
forming a plurality of active thermal sites at respective locations on the substrate and one or more passive thermal regions disposed between the plurality of active thermal sites on the substrate; wherein:
each active thermal site comprises a heating element configured to apply a variable amount of heat to a corresponding site of said medium and a thermal insulation layer disposed between the heating element and the substrate;
each passive thermal region comprises a thermal conduction layer configured to conduct heat from a corresponding portion of the medium to the substrate; and
the thermal conduction layer of said one or more passive thermal regions has a lower thermal resistance in a direction perpendicular to a plane of the substrate than the thermal insulation layer of said plurality of active thermal sites.

Preferably, in any embodiment of the process of the invention the solid substrate comprises a temperature control device for controlling temperatures at a plurality of sites of the solid substrate, comprising:
(A)
(i) a plurality of active thermal sites disposed at respective locations on the substrate, each active thermal site comprising a heating element configured to apply a variable amount of heat to a corresponding site of said medium and a thermal insulation layer disposed between the heating element and the substrate; and
(ii) one or more passive thermal regions disposed between the plurality of active thermal sites on the substrate, each passive thermal region comprising a thermal conduction layer configured to conduct heat from a corresponding portion of the medium to the substrate;
   wherein the thermal conduction layer of said one or more passive thermal regions has a lower thermal resistance in a direction perpendicular to a plane of the substrate than the thermal insulation layer of said plurality of active thermal sites.

Further embodiments of the temperature control device are set out in (B)-(R) below:
(B) The temperature control device may further comprise control circuitry configured to control whether a selected active thermal site provides heating of the corresponding site of the medium using the heating element or cooling of the corresponding site by heat flow through said thermal insulation layer to said substrate, in dependence on whether an amount of heat generated by the heating element of said selected active thermal site is greater or smaller than a threshold amount, preferably wherein the threshold amount is dependent on the thermal resistance of the thermal insulation layer in the direction perpendicular to the plane of the substrate
(C) The temperature control device wherein each active thermal site can comprise a temperature sensor configured to sense a temperature at the corresponding active thermal site
(D) The temperature control device of (C) may comprise a plurality of feedback loops each corresponding to a respective active thermal site;
each feedback loop configured to implement a transfer function for determining a target amount of heat to be applied to the corresponding site of the medium in dependence on the temperature sensed by the temperature sensor of the corresponding active thermal site and a target temperature specified for the corresponding site of the medium.
(E) The temperature control device of (D), wherein each feedback loop is configured to implement a lineariser function to map said target amount of heat determined by the transfer function to an input signal for controlling the heating element of the corresponding active thermal site.
(F) The temperature control device of (E) wherein the lineariser function is a function of the temperature sensed by the temperature sensor of the corresponding active thermal site.
(G) The temperature control device of (E) or (F) wherein the lineariser function determines the input signal in dependence on a sum of the target amount of heat and an amount of heat lost from the heating element of the active thermal site to the substrate and surrounding passive thermal regions.
(H) The temperature control device of any of (A)-(G), wherein the heating element comprises a resistive heating element.
(I) The temperature control device according to any of (A)-(H), wherein the thermal insulation layer of said plurality of active thermal sites has a greater thermal resistance in a direction parallel to the plane of the substrate than in a direction perpendicular to the plane of the substrate.
(J) The temperature control device of any of (A)-(I), wherein the thermal insulation layer of a given active thermal site comprises a thin film material having a thickness z in the direction perpendicular to the plane of the substrate which is substantially smaller than a smallest dimension L of the thermal insulation layer of the active thermal site in a direction parallel to the plane of the substrate.
(K) The temperature control device according to any of (A)-(I), wherein the thermal insulation layer comprises one or more voids.
(L) The temperature control device according (K), wherein the voids extend in a direction substantially perpendicular to the plane of the substrate.
(M) The temperature control device according to (K) or (L), wherein the thermal insulation layer comprises one or more pillars of a first thermal insulation material extending substantially perpendicular to the plane of the substrate in the area of the active thermal site between the heating element and the substrate, wherein said one or more voids are disposed between or around the pillars.
(N) The temperature control device according to any of (K) or (L), wherein the thermal insulation layer comprises a void extending throughout the entire area of the active thermal site between the heating element and the substrate.
(O) The temperature control device according to any of (A)-(N), comprising a cooling mechanism to cool the substrate to act as a heat sink.
(P) The temperature control device according to any of (A)-(O), wherein the medium comprises a fluid, and the temperature control device comprises a fluid flow control element configured to control flow of the fluid over the plurality of active thermal sites and the one or more passive thermal regions.
(Q) The temperature control device according to (P), wherein the active thermal sites are disposed in one or more rows oriented substantially parallel to the direction of fluid flow controlled by the fluid flow control element;
each row comprising two or more active thermal sites with a passive cooling region disposed between each pair of adjacent active thermal sites of the row.
(R) The temperature control device according to (P), wherein each active thermal site has a length along the row direction that is greater than a length along the row direction of each passive cooling region disposed between adjacent active thermal sites of the row.

The following examples are provided to further illustrate the invention.

The results of the time course studies show that it is possible to devise cleavable linkers or protecting groups for use in the invention, having a wide range of properties under different cleavage conditions. Thus, the linkers and protecting groups for use in the invention can be fine tuned to enable controlled cleavage and deprotection.

### EXAMPLES

### Analytical Methods

### LC-MS Methods

The time-course studies discussed below and analysis of reactions were carried out using LC-MS, which is described generally below:

Acquity Arc system; 2498 UV/Vis detector, QDa Detector Column; XSelect CSH C18 XP Column, 130A, 2.5 μm, 2.1 mm×50 mm

### Method A (Acidic)

Component 1: H₂O+0.1% formic acid

Component 2: MeCN (acetonitrile)

| Time/seconds | Component 1 | Component 2 |
|---|---|---|
| 0 | 95 | 5 |
| 120 | 5 | 95 |
| 150 | 5 | 95 |
| 156 | 95 | 5 |
| 240 | 95 | 5 |

### Method B (Basic)

Component 1: H₂O+0.1%-25% ammonium formate in H₂O

Component 2: MeCN

| Time/seconds | Component 1 | Component 2 |
|---|---|---|
| 0 | 95 | 5 |
| 120 | 5 | 95 |
| 150 | 5 | 95 |
| 156 | 95 | 5 |
| 240 | 95 | 5 |

### Method C (Long Acidic)

Component 1: H₂O+0.1% formic acid

Component 9: MeCN

| Time/seconds | Component 1 | Component 2 |
|---|---|---|
| 0 | 95 | 5 |
| 180 | 5 | 95 |
| 225 | 5 | 95 |
| 234 | 95 | 5 |
| 360 | 95 | 5 |

Route to compounds with protected activating group:

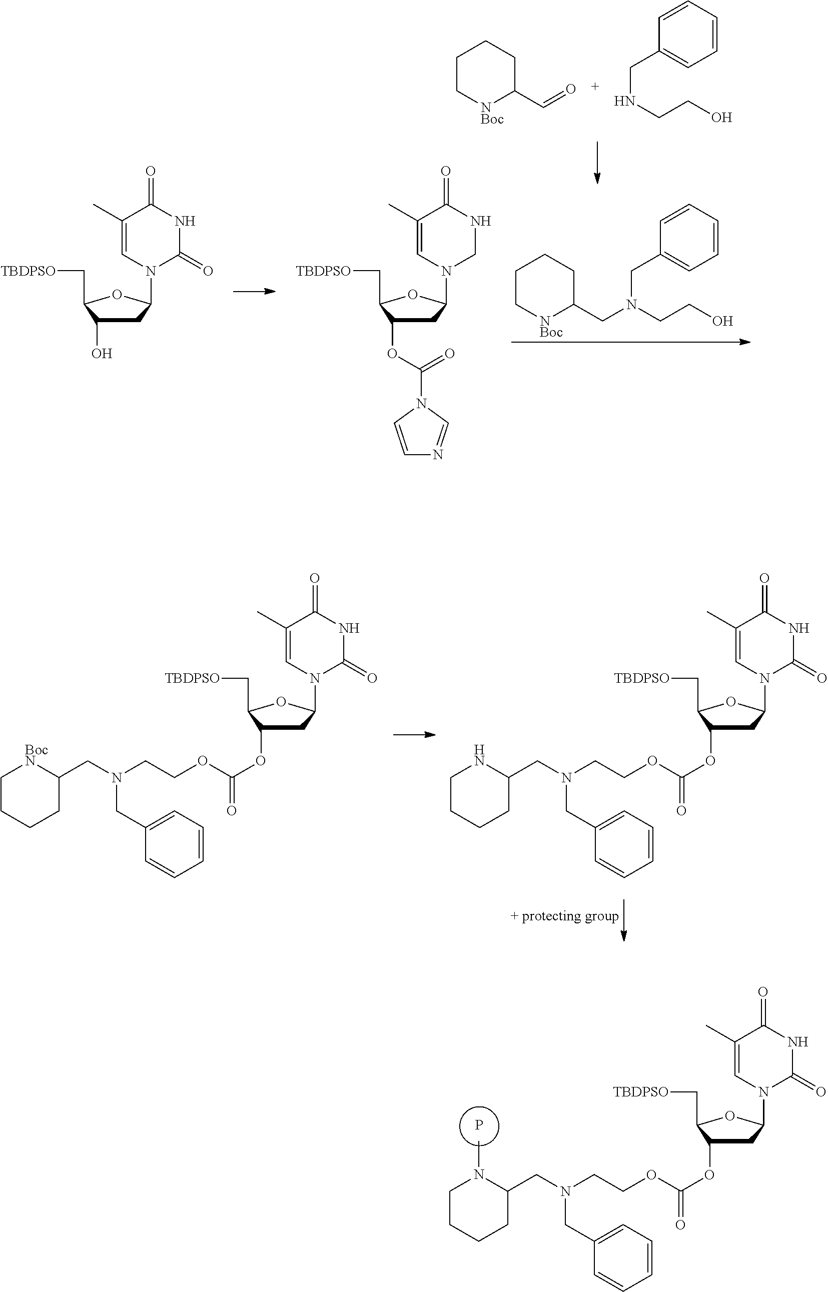

### Example 1

### Example 1A: tert-Butyl 2-((benzyl(2-hydroxyethyl)amino)methyl)piperidine-1-carboxylate (1)

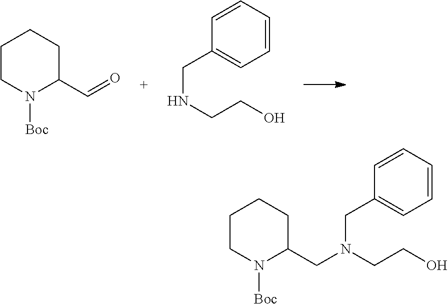 [SMILES: CC(C)(C)OC(=O)N1CCCCC1C=O ; OCCNCc1ccccc1 ; CC(C)(C)OC(=O)N1CCCCC1CN(CCO)Cc1ccccc1]

1-N-Boc-2-Piperidinecarbaldehyde (2 g, 9.3 mmol) was dissolved in THF (200 mL), and acetic acid (2.4 mL), and 2-benzylaminoethanol (1.6 g, 10 mmol, 1.2 eq.) were added. After 10 minutes at room temperature, sodium triacetoxyborohydride was added and the solution was stirred overnight. Saturated aqueous NaHCO₃ (300 mL) and ethyl acetate (500 mL) were added and the layers were separated. The organic layer was dried (MgSO₄) and the solvent was removed under reduced pressure. The residue was purified by silica chromatography (0-10% MeOH-DCM) to give the product as a colourless oil (2.33 g, 71%. LC-MS Method B (Basic); Rt=1.60, m/z 349.2 (MH⁺).

### Example 1B: tert-Butyl 2-((benzyl(2-(((((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl)oxy)carbonyl)oxy)ethyl)amino)methyl)piperidine-1-carboxylate (2)

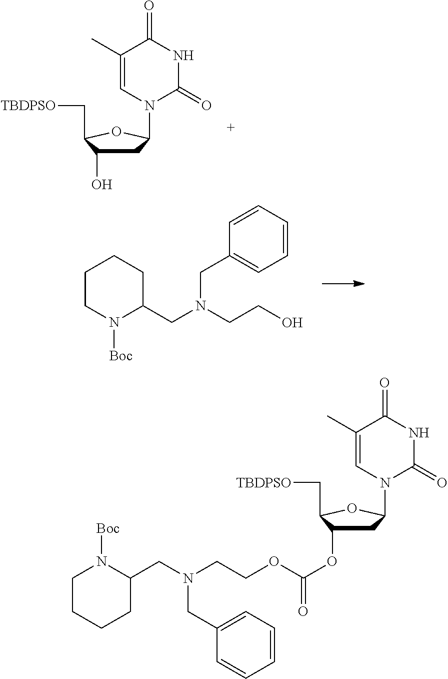

5′-O-TBDPS-Thymidine (1.3 g, 2.9 mmol, 1.0 eq) and CDI (534 mg, 3.5 mmol, 1.2 eq) were dissolved in anhydrous acetonitrile (40 mL) and the solution was stirred at room temperature under N₂ overnight. After this time, the reaction was complete by tlc (10% MeOH-DCM, uv). tert-Butyl 2-((benzyl(2-hydroxyethyl)amino)methyl)piperidine-1-carboxylate (1) (1 g, 2.29 mmol) and 1,1,3,3-tetramethylguanidine (0.72 mL, 5.8 mmol, 2 eq.) were then added and the solution was stirred for a further two hours. Water (200 mL) and EtOAc (200 mL) were added and the layers were separated. The organic layer was dried (MgSO₄) and the solvent was removed. The resulting oil was purified by silica chromatography, eluting with 0-50% EtOAc-petrol to give the product as a colourless oil, 890 mg, 37%. LC-MS; Method B (Basic); Rt=3.47, m/z 855.46 (MH⁺).

### Example 1C: 2-(Benzyl(piperidin-2-ylmethyl)amino)ethyl ((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl) carbonate (3)

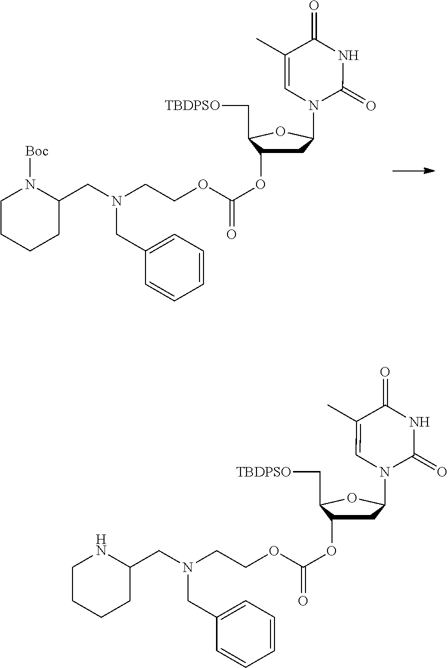

tert-Butyl 2-((benzyl(2-(((((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl)oxy)carbonyl)oxy)ethyl)amino)methyl)piperidine-1-carboxylate (2) (100 mg, 0.12 mmol) was dissolved in dichloromethane (2 mL) and trifluoroacetic acid (2 mL) and the solution was stirred at room temperature for 1 h. After this time, the reaction was complete by LC-MS. The solvent was removed and dichloromethane (100 mL) and saturated aqueous NaHCO₃ (100 mL) were added and the layers were separated. The organic layer was dried (MgSO₄) and the solvent was removed under reduced pressure at 20° C. The residue was purified by silica chromatography (0-10% MeOH-DCM) to give the product as a colourless oil (63 mg, 71%). LC-MS; Method B (Basic); Rt=2.20, m/z 755.46 (MH⁺).

### Example 2

### (1,1-Dioxidobenzo[b]thiophen-2-yl)methyl 2-((benzyl(2-(((((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl)oxy)carbonyl)oxy)ethyl)amino)methyl)piperidine-1-carboxylate (4)

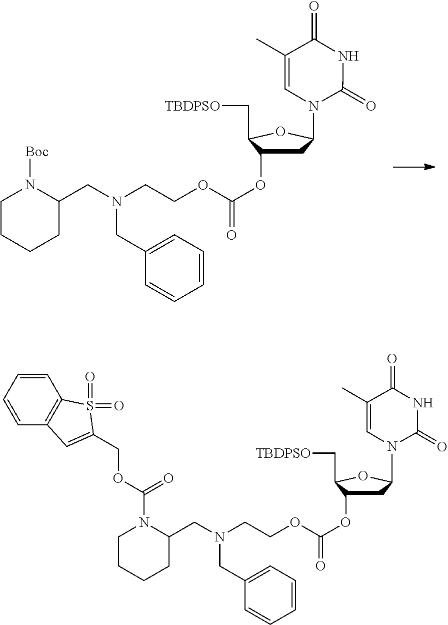

tert-Butyl 2-((benzyl(2-(((((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl)oxy)carbonyl)oxy)ethyl)amino)methyl)piperidine-1-carboxylate (2) (290 mg, 0.38 mmol) was dissolved in 1:1 TFA-DCM (10 mL) at rt. After 1 h the reaction was complete by tlc (10% MeOH-DCM, uv). The excess TFA was removed under reduced pressure and sat. aq. NaHCO₃ (50 mL) and DCM (50 mL) were added. The layers were separated and the organic layer was washed with brine (50 mL), dried (MgSO₄) and the solvent removed to give the free amine as a colourless oil. This oil was dissolved in DCM (20 mL) and Hunig's base (0.13 mL, 0.76 mmol, 2 eq.) was added, followed by 1,1-dioxobenzo[b]thiophen-2-ylmethyl chloride (100 mg, 0.46 mmol, 1.2 eq.). After 1 h the reaction was complete by tlc, (10% MeOH-DCM). Water (50 mL) and DCM (50 mL) were added, the layers were separated and the organic layer was dried (MgSO₄). The solvent was removed under reduced pressure and the crude product was purified by silica chromatography (0-60% EtOAC-petrol) to give the product as a colourless oil (250 mg, 67%). LC-MS; Method B (Basic); Rt=2.85, m/z 977.40 (MH⁺).

### Example 3

### (9H-fluoren-9-yl)methyl 2-((benzyl(2-(((((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3 yl)oxy)carbonyl)oxy)ethyl)amino)methyl)piperidine-1-carboxylate (5)

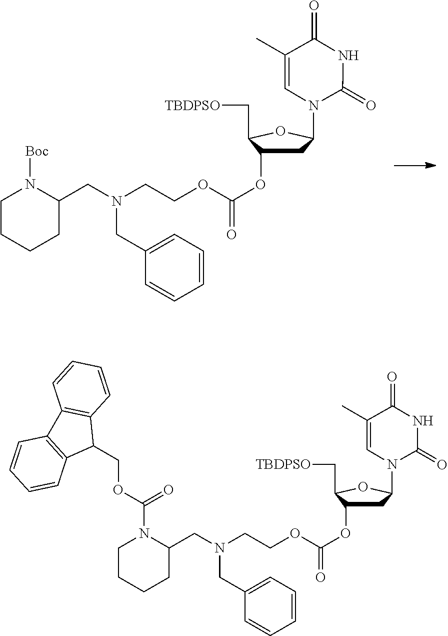

tert-Butyl 2-((benzyl(2-(((((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl)oxy)carbonyl)oxy)ethyl)amino)-methyl)piperidine-1-carboxylate (2) (800 mg, 0.93 mmol) was dissolved in 1:1 TFA-DCM (20 mL) at rt. After 1 h the reaction was complete by tlc (10% MeOH-DCM, uv). The excess TFA was removed under reduced pressure and sat. aq. NaHCO₃ (50 mL) and DCM (50 mL) were added. The layers were separated and the organic layer was washed with brine (50 mL), dried (MgSO₄) and the solvent removed to give the free amine as a colourless oil. This oil was dissolved in DCM (60 mL) and Hunig's base (0.26 mL, 1.86 mmol, 2 eq.) was added, followed by 9-fluorenylmethoxycarbonyl chloride (342 mg, 1.1 mmol, 1.2 eq.). After 1 h the reaction was complete by tlc, (10% MeOH-DCM). Water (50 mL) and DCM (50 mL) were added, the layers were separated and the organic layer was dried (MgSO₄). The solvent was removed under reduced pressure and the crude product was purified by silica chromatography (0-60% EtOAC-petrol) to give the product as a colourless oil (250 mg, 67%). LC-MS; Method B (Basic); Rt=3.31, m/z 978.61.40 (MH⁺).

### Example 4

### Example 4A: tert-Butyl 2-((benzyl(2-hydroxyethyl)amino)methyl)pyrrolidine-1-carboxylate (6)

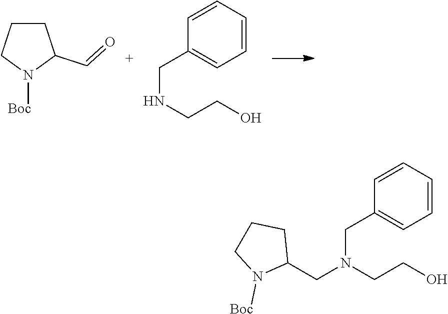 [SMILES: CC(C)(C)OC(=O)N1CCCC1C=O ; OCCNCc1ccccc1 ; CC(C)(C)OC(=O)N1CCCC1CN(CCO)Cc1ccccc1]

N-Boc-L-Prolinal (3 g, 15 mmol) was dissolved in THF (200 mL), and acetic acid (3 mL 75 mmol, 5 eq.) and 2-benzylaminoethanol (2.3 g, 16 mmol, 1.2 eq.) were added. After 10 minutes at rt, sodium triacetoxyborohydride was added and the solution was stirred for 3 h. Sat. aq. NaHCO₃ was added and the layers were separated. The organic layer was dried (MgSO₄) and the solvent was removed under reduced pressure. The residue was purified by silica chromatography (0-10% MeOH-DCM) to give the product as a colourless oil (3.1 g mg, 63%). LC-MS; Method B (Basic); Rt=1.53, m/z 335.3 (MH⁺).

### Example 4B: tert-Butyl (S)-2-((benzyl(2-(((((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl)oxy)carbonyl)oxy)ethyl)amino)methyl)pyrrolidine-1-carboxylate (7)

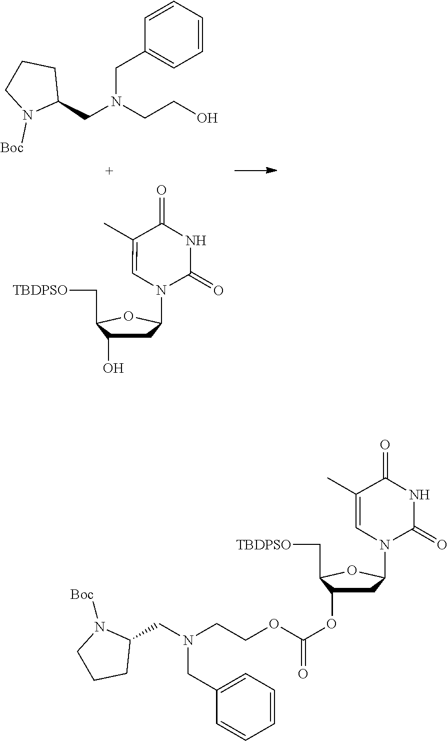

5′-O-TBDPS-Thymidine (1.4 g, 3 mmol, 1.0 eq.) and CDI (530 mg, 3.3 mmol, 1.2 eq.) were dissolved in anhydrous acetonitrile (40 mL) and the solution was heated at 40° C. under N₂ for 2 hours. tert-Butyl 2-((benzyl(2-hydroxyethyl)amino)methyl)pyrrolidine-1-carboxylate (6) (1 g, 3 mmol) and 1,1,3,3-tetramethylguanidine (1 mL, 8.4 mmol, 3 eq.) were added and the solution was stirred at rt for 2 h. Water (200 mL) and EtOAc (200 mL) were added and the layers were separated. The organic layer was dried (MgSO₄) and the solvent was removed. The resulting oil was purified by silica chromatography, eluting with 0-50% EtOAc-petrol to give the product as a colourless oil, 1 g, 40%. LC-MS; Method B (Basic); Rt=3.30, m/z 841.09 (MH⁺).

### Example 4C: 2-(Benzyl(((S)-pyrrolidin-2-yl)methyl)amino)ethyl ((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl) carbonate

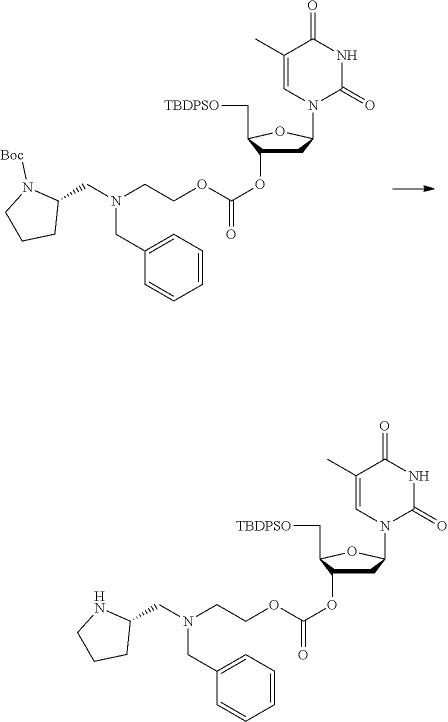

tert-Butyl (S)-2-((benzyl(2-(((((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl)oxy)carbonyl)oxy)ethyl)amino)methyl)pyrrolidine-1-carboxylate (7) (100 mg, 0.12 mmol) was dissolved in dichloromethane (2 mL) and trifluoroacetic acid (2 mL) and the solution was stirred at room temperature for 1 h. After this time, the reaction was complete by LC-MS. The solvent was removed and dichloromethane (100 mL) and saturated aqueous NaHCO₃ (100 mL) were added and the layers were separated. The organic layer was dried (MgSO₄) and the solvent was removed under reduced pressure. The residue was purified by silica chromatography (0-10% MeOH-DCM) to give the product as a colourless oil (60 mg, 69%).

### Example 5—Time-Course Experiments

### General Procedure—Reactions at High Temperature

The compound to be tested was dissolved in the required solution at a concentration of 0.5 mg/mL at room temperature. This solution was divided (0.5-0.75 mL per vial) between enough LC-MS vials to measure the reaction course at the required number of time-points as well as one for the room temperature measurements. The vials for the heated experiment were immediately placed in a hot water bath set at 90° C. (±0.1° C.), such that the level of liquid in the vial was below the surface of the hot water. At each time-point in the experiment an LC-MS vial was removed and then immediately cooled in a brine ice-bath at a temperature of 3° C. The LC-MS experiment was then carried out within ten minutes of the reaction being quenched by cooling. The ratio of starting material to cleaved TBDPS-Thymidine was measured by integrating the corresponding peaks in the UV trace of the LC-MS.

### General PROCEDURE—Reactions at Room Temperature

An LC-MS vial containing the same solution as used for the high-temperature experiment was kept at room temperature i.e. 20±3° C. and the solution was analysed by LC-MS at suitable time-points.

### Reactions at 10′C

An LC-MS vial containing the same solution as used for the high-temperature experiment was place in the pre-chilled auto sampler chamber of the LC-MS machine set at 10° C. and the solution was repeatedly analysed by LC-MS at suitable time-points.

### Example 5A: Time Course Studies on the Cleavage of the Unprotected Linker of Example 1C

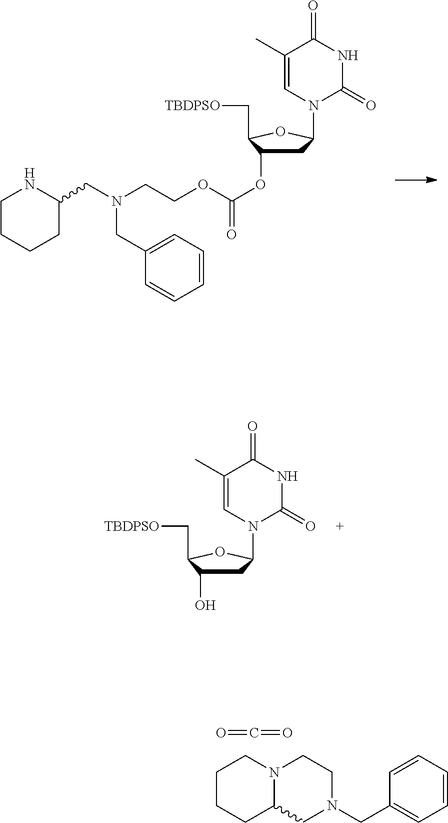

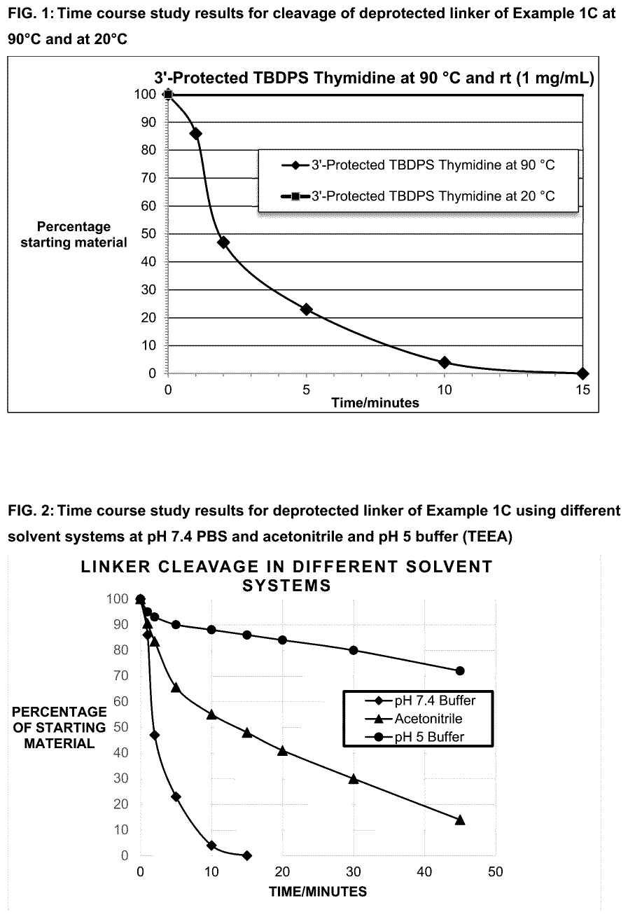

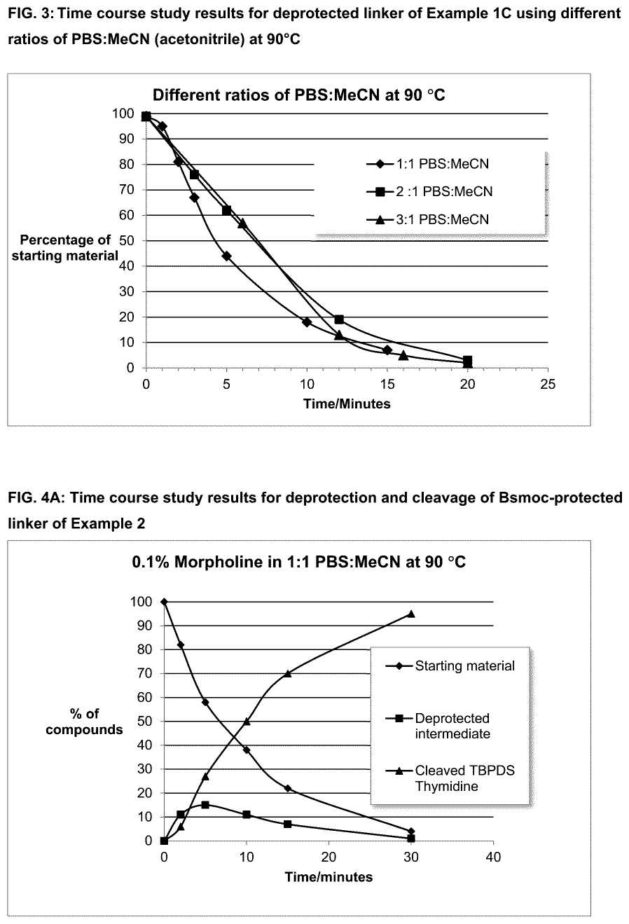

Time course studies on the cleavage of the above (unprotected) linker from TBDMS-protected thymidine, was carried out according to the general procedures above:
(i) at 90° C. and at 20° C. (room temperature) (FIG. 1) [1:1 pH 7.4 PBS (phosphate buffered saline) and acetonitrile];
(ii) using different solvent systems [pH 7.4 PBS (phosphate buffered saline)]; acetonitrile and pH 5 buffer (TEEA (triethylammonium acetate) buffer] (FIG. 2)
(iii) using different ratios of PBS:MeCN (acetonitrile) at 90° C. (FIG. 3)

The results of the time course studies on the above compound in PBS (phosphate buffered saline) and MeCN (acetonitrile) at 90° C. and at room temperature (20° C.) are shown in FIG. 1.

As shown in FIG. 1, the unprotected linker shows a clear differentiation of cleavage at 20° C. vs 90° C. Thus, at 20° C., only starting material was detected, whereas at 90° C., the linker was cleaved from the nucleotide. Further, at 90° C., the cleavage was rapid, with no starting material remaining after about 13 minutes.

### Example 5B: Studies on Deprotection of Bsmoc-Protected Activating Group Followed by Linker-Cleavage (Compound of Example 2)

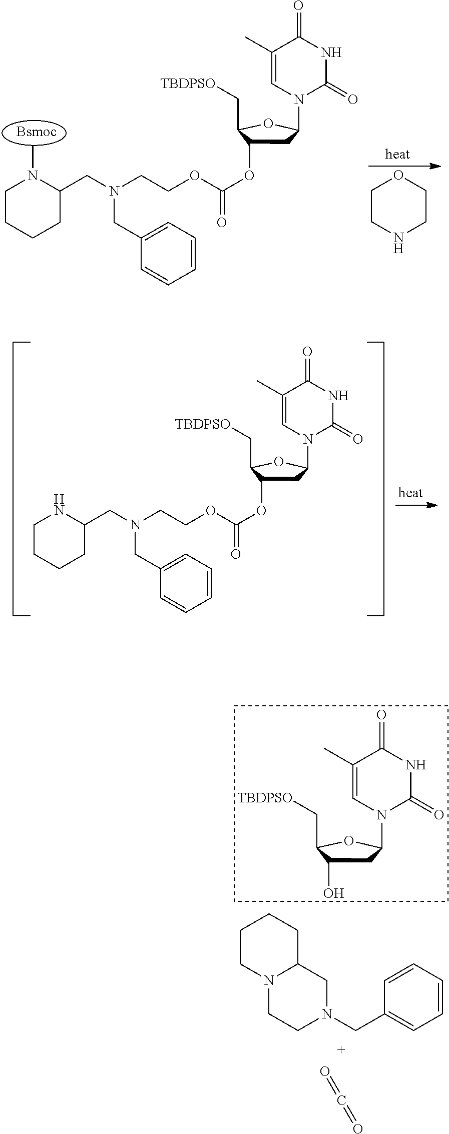

Time course studies on the cleavage of the above Bsmoc-protected linker from TBDMS-protected thymidine (i.e. the compound of Example 2), was carried out according to the general procedures above.

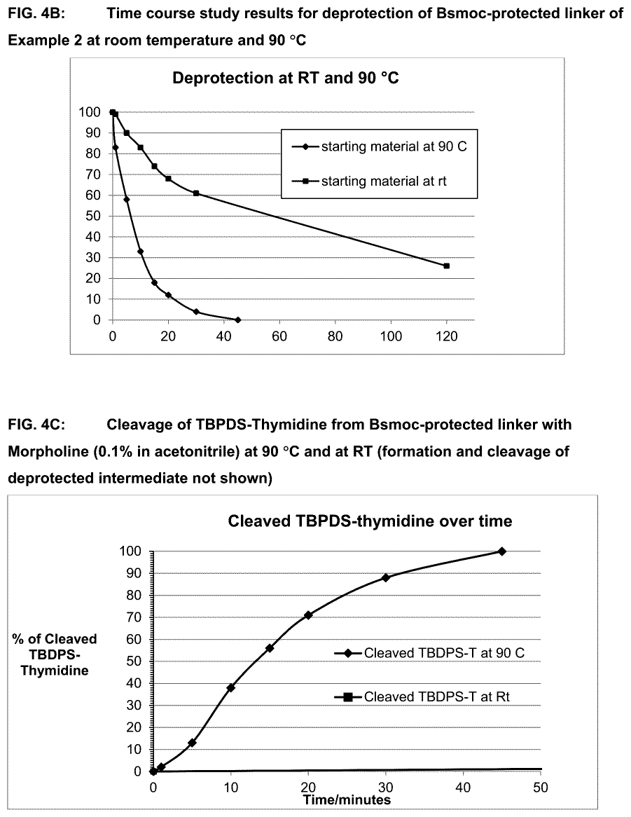

The Bsmoc protecting group was heated at 90° C. and the concentrations of the starting material, Bsmoc-deprotected intermediate and the cleaved thymidine were measured (FIG. 4A). FIG. 4B shows the extent of deprotection of the Bsmoc-group over time at 20° C. and at 90° C.

FIG. 4C shows that when the Bsmoc-protected linker is treated with base at room temperature this does not lead to immediate cleavage of the linker, because although the deprotection step occurs reasonably quickly at RT, the second step (i.e. cleavage) requires heating.

### Example 5C: Stability Studies on Bsmoc-Protected Linker of Example 2

Stability studies were conducted under different pH conditions at 80° C.:
(i) pH 7.4 phosphate buffered saline;
(ii) pH 9 phosphate buffered saline
(iii) pH 5 TEEA (triethylammonium acetate) buffer

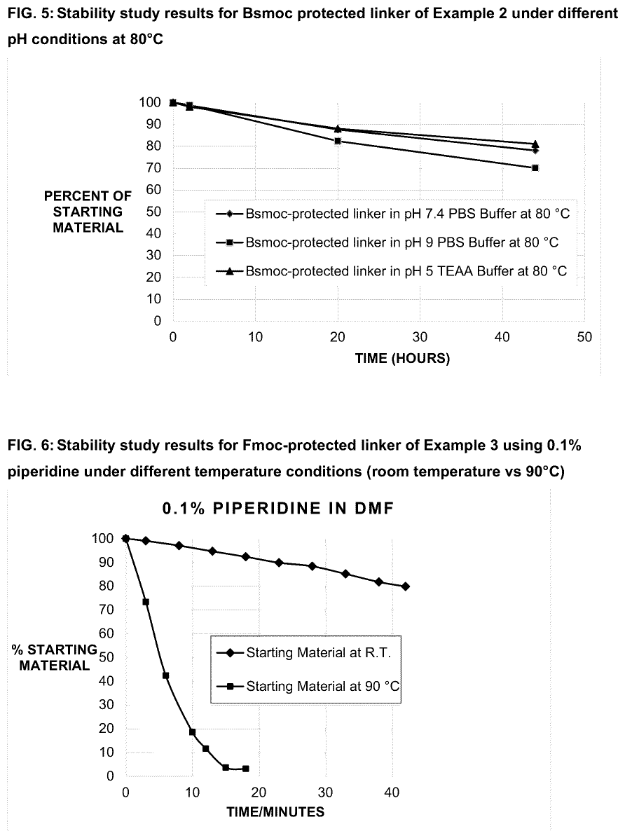

The results are shown in FIG. 5. The results show that minimal cleavage of the Bsmoc-protected linker was observed under heated (80° C.) conditions over several hours. Furthermore, minimal side-product formation was observed under these conditions. By comparison, as shown in the previous study (FIGS. 4A, 4B and 4C), conditions of 0.1% morpholine at 90° C. enabled a facile and rapid two step deprotection and linker cleavage.

### Example 5D: Studies on Deprotection of Fmoc-Protected Activating Group Followed by Linker-Cleavage (Compound of Example 3)

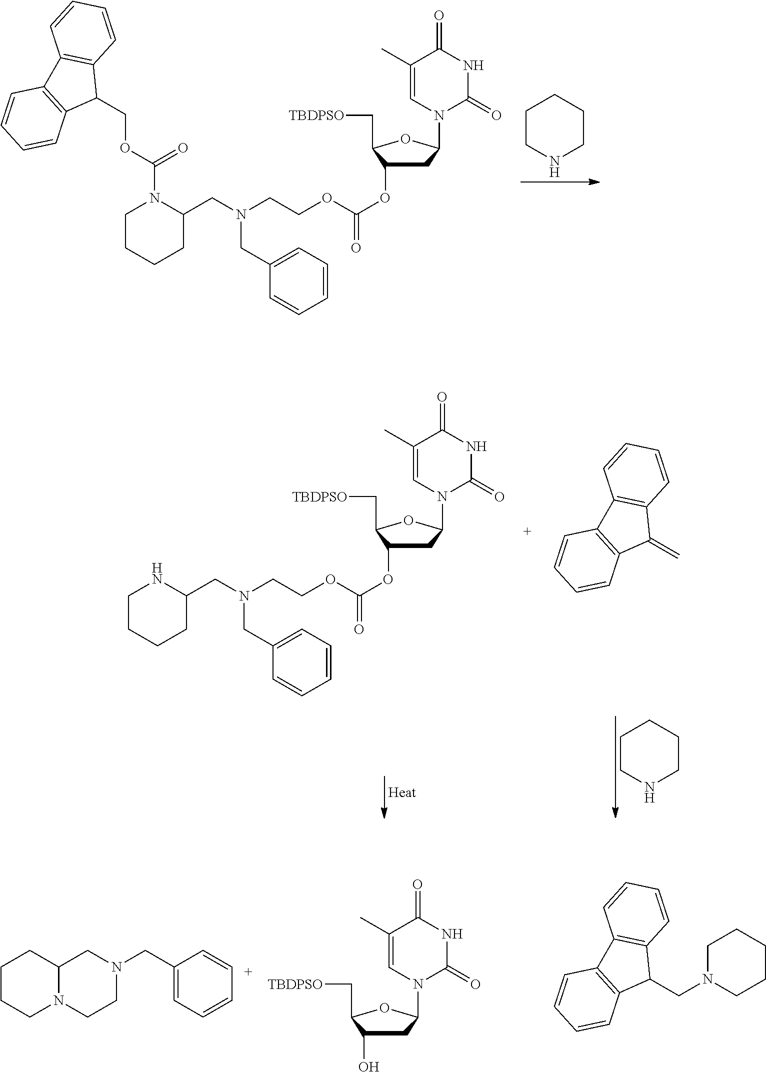

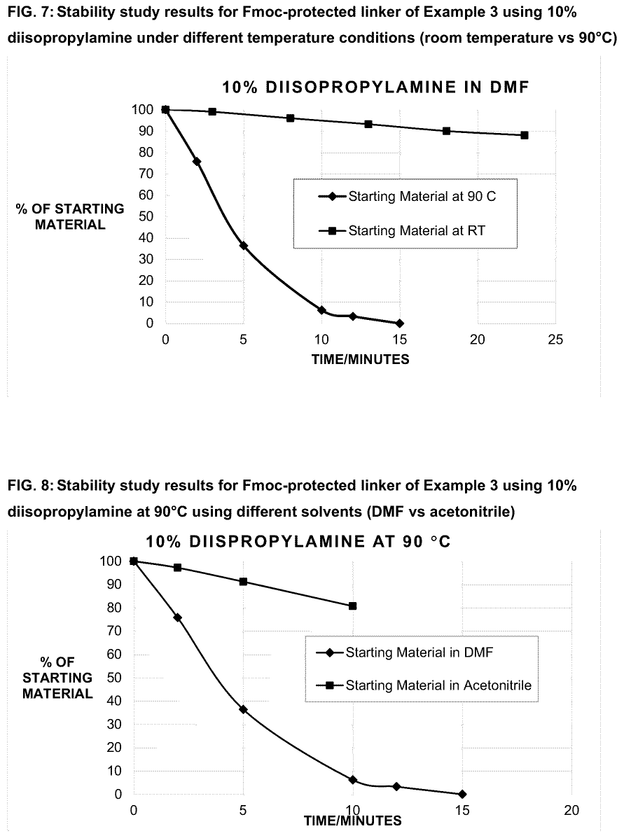

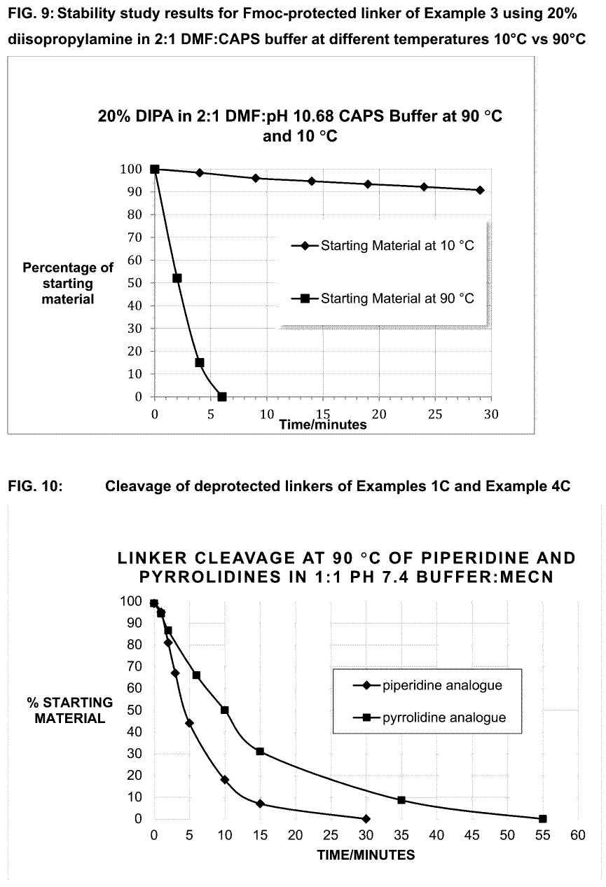

Studies with the Fmoc protecting group of the compound of Example 3 demonstrated a similar level of control to the Bsmoc protecting group. A key difference was that, as well as piperidine, non-nucleophilic bases such as diisopropylamine can also be used to remove the Fmoc group (FIGS. 6, 7 and 9). A significant deceleration of the reaction rate was observed when changing the solvent from DMF to acetonitrile (FIG. 8). Hence, it can be seen that the incorporation of different protecting groups offers further control of the deprotection-cleavage conditions. In addition, adjustment of the reaction conditions enables a simple way of fine tuning the deprotection and cleavage of the linker.

### Example 5E: Comparative Studies Between the Pyrrolidine (Compound of Example 4C) and Piperidine (Compound of Example 1C) Activating Groups

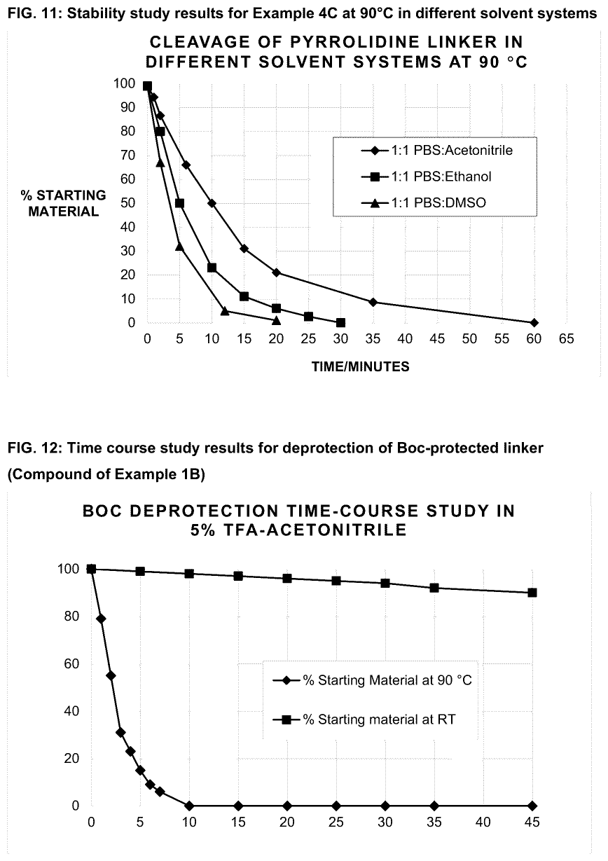

Studies with a pyrrolidine activating group showed that there was a deceleration of reaction speed compared to the piperidine activating, in spite of the increased nucleophilicity of the pyrrolidine nitrogen (FIG. 10). These studies indicate that the conformation of the cyclised product is likely to be more important in determining reaction speed, and thus provides a further method by which fine control of the linker deprotection-cleavage can be achieved.

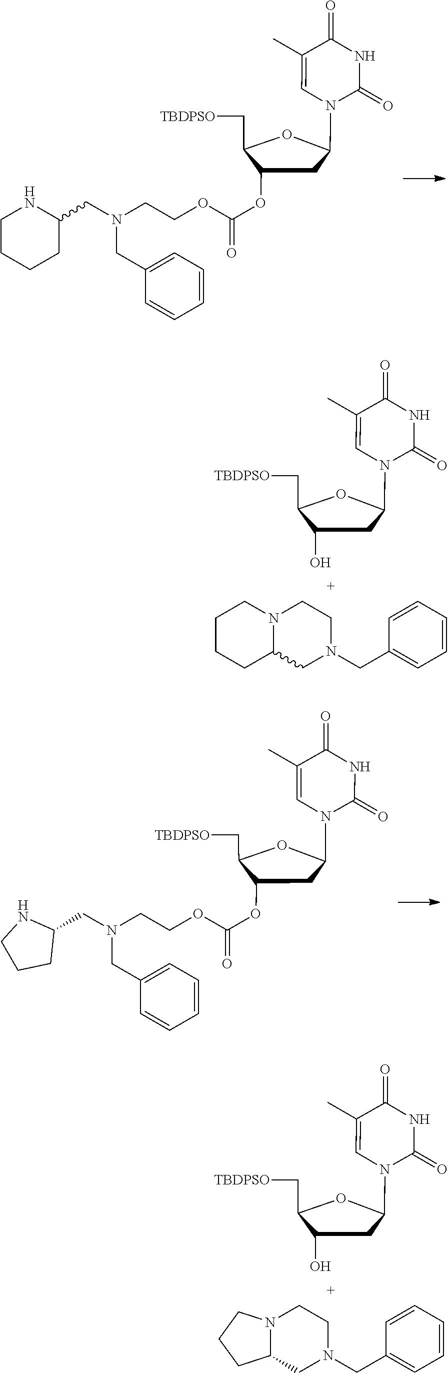

### Example 5F: Co-Solvent Study with Pyrrolidine Linker (Compound of Example 4C)

The effect of co-solvents on reaction speed was explored. As shown in FIG. 11, it was found that DMSO gave the fastest reaction speed in this system.

### Example 5G: Time-Course Study for Deprotection of Boc-Protected Linker (Compound of Example 1B)

The general procedure for carrying out the time-course experiments at 20° C. and at 90° C. was used, at with the modification that the reaction at each time-point was quenched by cooling the LC-MS vial in a brine-ice bath, and then adding an excess of triethylamine (50 μL, ˜3 eq.)

The aim of using an acid-cleaved protecting group was to demonstrate a two step deprotection-cleavage process with a different level of orthogonality in each step, since deprotection of the activating group with acids leads to a protonated activating group which is unable to effect linker cleavage until deprotonation occurs. The study demonstrated that no linker-cleavage was observed under these conditions despite 100% deprotection of the activating group occurring (FIG. 12).

### Example 6—Synthesis of c-Carbon Substituted Compounds

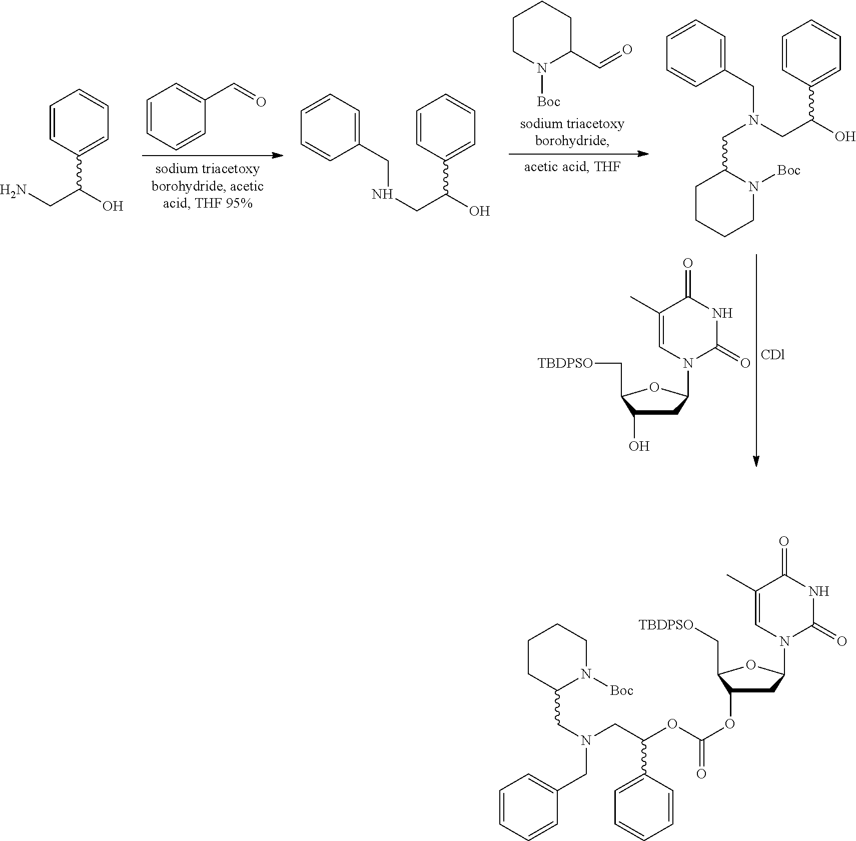

### Example 6A: 2-(Benzylamino)-1-phenylethan-1-ol

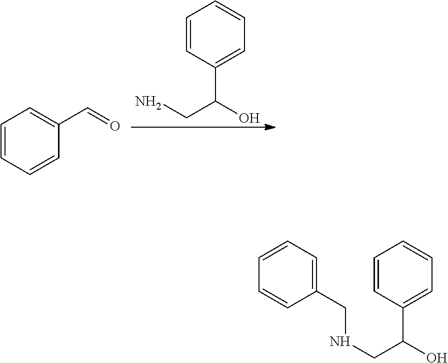 [SMILES: O=Cc1ccccc1 ; NCC(O)c1ccccc1 ; OC(CNCc1ccccc1)c1ccccc1]

2-Hydroxy-2-phenylethylamine (4.7 g, 34 mmol, 1.2 eq) and benzaldehyde (3.6 g, 34 mmol) were dissolved in methanol (100 ml) and stirred at rt for 10 minutes. After this time, the solution was cooled to 0° C. and sodium borohydride (1.6 g, 34 mmol) was added. The solution was warmed to rt and stirred for 2 h, after which time the reaction was complete by LC-MS and tlc. An ethyl acetate-water workup was carried out and the organic layer was dried (MgSO₄) and the solvent was removed under reduced pressure to give an off-white crystalline solid. This was triturated with petrol and ethyl acetate to give a white solid, 5 g, 65%. LC-MS; Method B (Basic); Rt=1.94, m/z 228.2 (MH⁺).

### Example 6B: tert-Butyl 2-((benzyl(2-hydroxy-2-phenylethyl)amino)methyl) piperidine-1-carboxylate

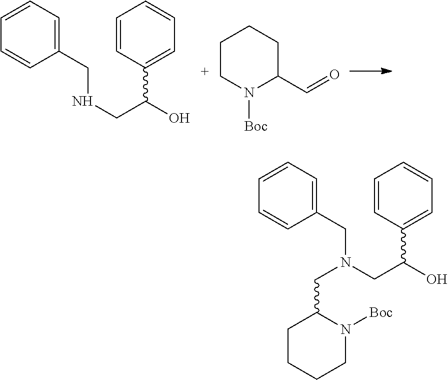

2-(Benzylamino)-1-phenylethan-1-ol (1.96, 8.6 mmol) and 1-N-boc-2-piperidinecarbaldehyde (1.8 g, 8.6 mmol) were dissolved in 1,2-dichloroethane (100 mL) and acetic acid was added (3 mL). After 10 minutes sodium triacetoxyborohydride (2.7 g, 12 mmol, 1.5 eq) was added and the solution was stirred overnight. After this time there was clean conversion to the product by LC-MS. A dichloromethane/saturated aqueous NaHCO₃ workup was carried out and the organic solution was dried (MgSO₄) and the solvent removed to give the diastereomeric products as a colourless oil, 3.7 g, 100%. LC-MS; Method B (Basic); Rt=2.88 and 2.93, m/z 425.3 (MH⁺).

### Example 6C: tert-Butyl 2-((benzyl(2-(((((2R,3S,5R)-2-(((tert-butyldiphenylsilyl)oxy)-methyl)-5-(5-methyl-2,4-dioxo-3,4-dihydropyrimidin-1(2H)-yl)tetrahydrofuran-3-yl)oxy)carbonyl)oxy)-2-phenylethyl)amino)methyl)piperidine-1-carboxylate

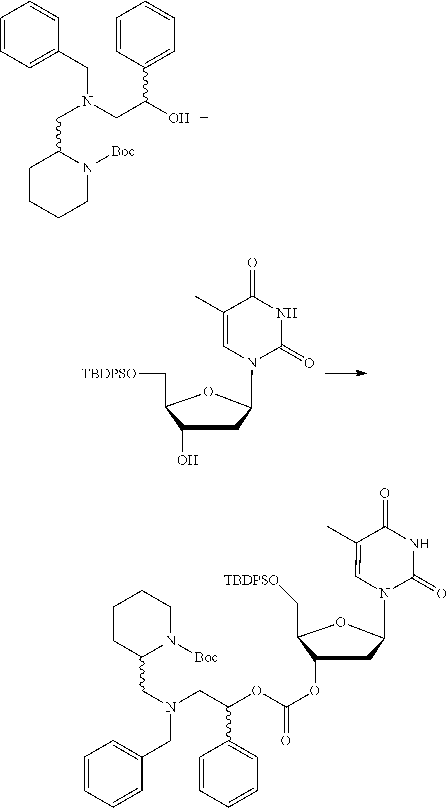

i) A mixture of tert-butyl 2-((benzyl(2-hydroxy-2-phenylethyl)amino)methyl)piperidine-1-carboxylate (500 mg, 1.2 mmol) and CDI (283 mg, 1.44 mmol, 1.2 eq.) were dissolved in anhydrous acetonitrile (60 mL) and the solution was heated at 50° C. for 1 h under N₂ to give clean conversion to the diastereomeric intermediates by LC-MS. ii) 5′-O-TBDPS-Thymidine (560 mg, 1.2 mmol) and DBU (0.44 mL, 2.92 mmol, 2 eq.) were then added and the solution was stirred overnight. A water/EtOAc/brine workup was carried out, the organic solution was dried (MgSO₄) and the solvent was removed under reduced pressure. The product was then purified by silica chromatography, eluting with 0-60% ethyl acetate-petrol to give the diastereomeric products as a white foam, 700 mg, 63%. LC-MS; Method C (Long Acidic); Rt=2.88 and 2.93, m/z 931.6 (MH⁺). ¹H NMR (CDCl₃) consistent with structure.

### Example 6D: 2-(benzyl(piperidin-2-ylmethyl)amino)-1-phenylethyl ((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl) carbonate

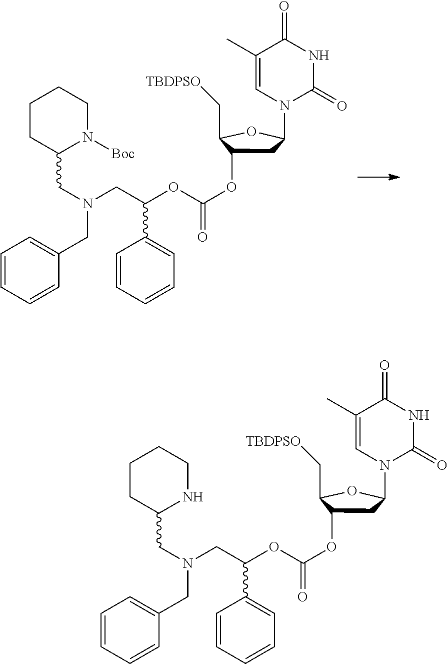

tert-Butyl 2-((benzyl(2-(((((2R,3S,5R)-2-(((tert-butyldiphenylsilyl)oxy)-methyl)-5-(5-methyl-2,4-dioxo-3,4-dihydropyrimidin-1(2H)-yl)tetrahydrofuran-3-yl)oxy)carbonyl)oxy)-2-phenylethyl)amino)methyl)piperidine-1-carboxylate (200 mg, 0.21 mmol) was dissolved in 2:1 DCM:TFA (5 mL) and the solution was stirred at rt for 3 hours. After this time saturated aqueous sodium bicarbonate (3×50 mL) was added and the layers were separated. The organic layer was dried (MgSO₄) and the solvent was removed under reduced pressure at 20° C. to give the diastereomeric products as a white foam, 160 mg, 88%. LC-MS; Method C (Long Acidic); Rt=2.31, 2.33 and 2.36, m/z 831.6 (MH⁺). ¹H NMR (CDCl₃) consistent with structure.

### Example 7—Time-Course Study on Non-Protected α-Phenyl Safety-Catch Linker (Compound of Example 6D)

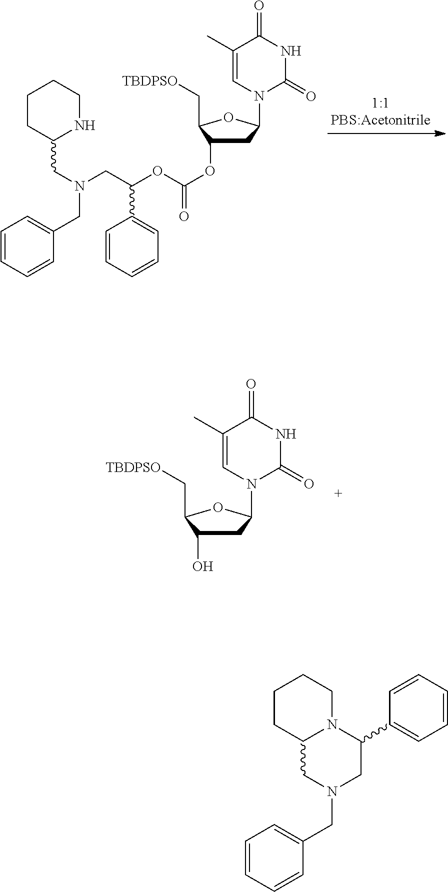

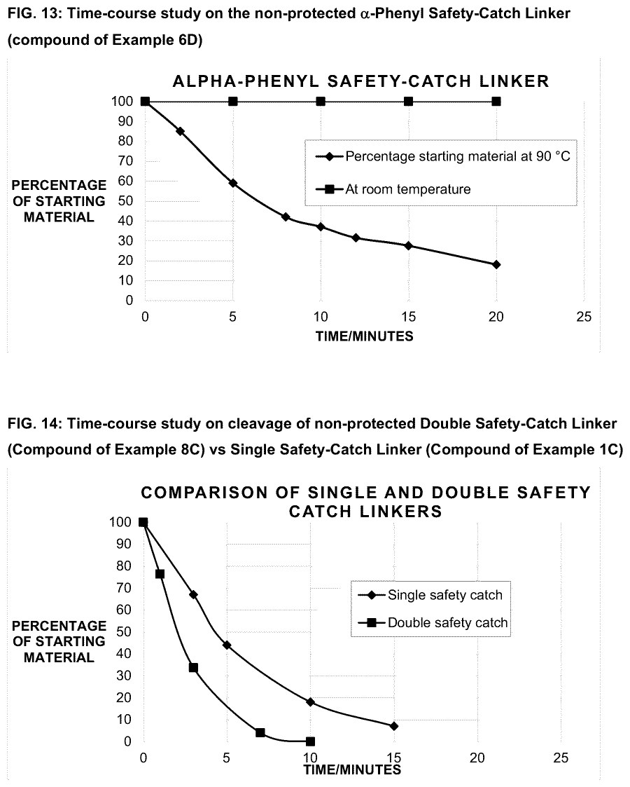

The aim of this study was to determine whether substitution at the α-carbon would be tolerated. It was observed that the reaction was slower than for the unsubstituted analogue, but proceeded cleanly (FIG. 13). Therefore the presence of substituents can be used to provide additional control of the rate of cleavage of the linkers/protecting groups.

### Example 8—Double Safety-Catch Protecting Groups

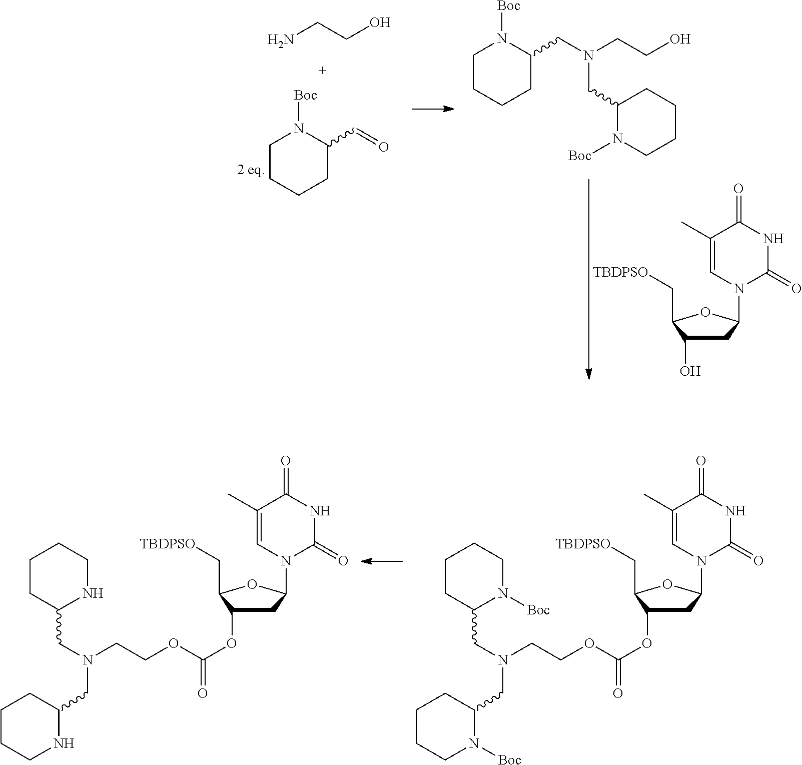

### Example 8A: di-Tert-butyl 2,2′-(((2-hydroxyethyl)azanediyl)bis(methylene))bis(piperidine-1-carboxylate)

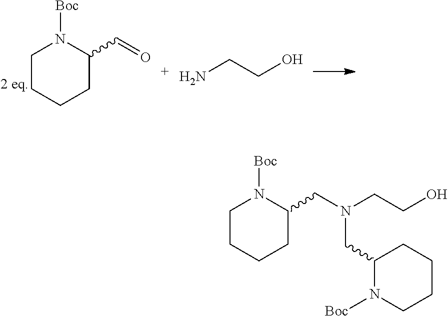

1-N-Boc-2-piperidinecarbaldehyde (1 g, 2.3 mmol) and ethanolamine (0.143 mL, 2.3 mmol) were dissolved in 1,2-dichloroethane (100 mL) and acetic acid (6 mL, 85 mmol, 5 eq.) was added. After 10 minutes sodium triacetoxyborohydride (2.7 g, 3.45 mmol, 1.5 eq.) was added. After 1 h, a mixture of both the intermediate and product were visible by LC-MS. Hence, the other equivalent of aldehyde was added, followed by another equivalent of sodium triacetoxyborohydride and the solution was then stirred overnight. A saturated aqueous NaHCO₃/DCM workup was carried out, the organic solution was dried (MgSO₄) and the solvent was removed under reduced pressure. The crude product was then purified by silica chromatography, eluting with DCM-EtOAc (0-50%) to give the product as a pale yellow oil, 1 g, 95%. LC-MS; Method A (Acidic); Rt=1.84, m/z 456.4 (MH⁺).

### Example 8B: di-Tert-butyl 2,2′-(((2-(((((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl)oxy)carbonyl)oxy)ethyl)azanediyl)bis(methylene))-bis(piperidine-1-carboxylate)

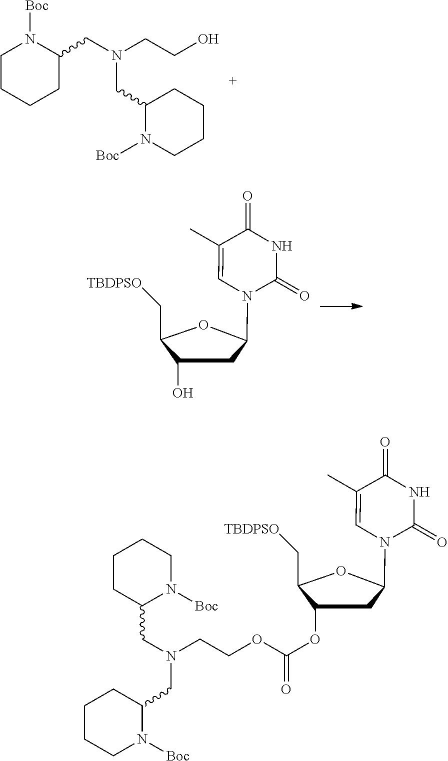

i) 5′-O-TBPDS-Thymidine (340 mg, 0.71 mmol) and CDI (130 mg, 0.85 mmol, 1.2 eq.) were dissolved in dry acetonitrile (60 mL) and the solution was heated at 50° C. for 4 hours and then left over the weekend. ii) di-Tert-butyl 2,2′-(((2-hydroxyethyl)azanediyl) bis(methylene))bis(piperidine-1-carboxylate) (323 mg, 0.71 mmol) and DBU (0.2 mL, 1.42 mmol, 2 eq.) were added and the solution was stirred at 40° C. for 2 h, after which time the reaction had gone to completion by LC-MS. A water/EtOAc workup was carried out and the organic solution was dried (MgSO₄) and the solvent was removed under reduced pressure. The residue was purified by silica chromatography, eluting with 0-80% EtOAc in DCM to give the product as a white foam, 280 mg, 41%. LC-MS; Method C (long acidic) Rt=4.03, m/z 962.7 (MH⁺).

### Example 8C: 2-(Bis(piperidin-2-ylmethyl)amino)ethyl ((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl) carbonate

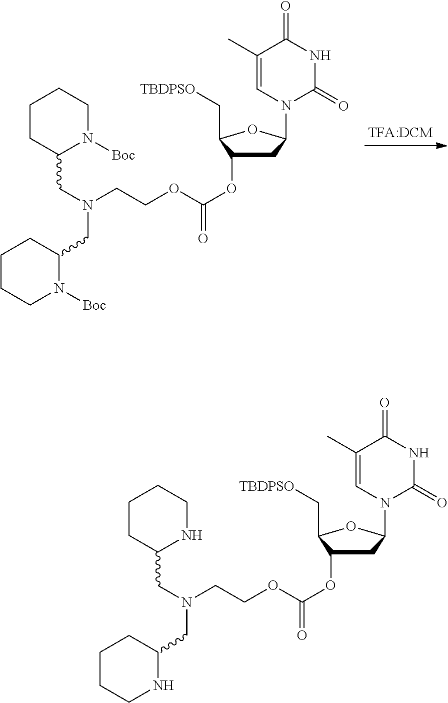

di-Tert-butyl 2,2′-(((2-(((((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl)oxy)carbonyl)oxy)ethyl) azanediyl)bis(methylene))bis(piperidine-1-carboxylate) (50 mg, 0.052 mmol) was dissolved in 1:1 TFA:DCM (2 mL) at rt. After 30 minutes the reaction was complete by LC-MS. DCM and saturated aqueous NaHCO₃ were added and the layers were separated. The DCM layer was dried (MgSO₄) and the solvent was removed under reduced pressure at 20° C. to give the product as a white foam, 30 mg, 76%. LC-MS; Method A (Acidic); Rt=1.73, m/z 762.4 (MH⁺).

### Example 9—Time-Course Study of Double Safety-Catch Linker (Compound of Example 8C)

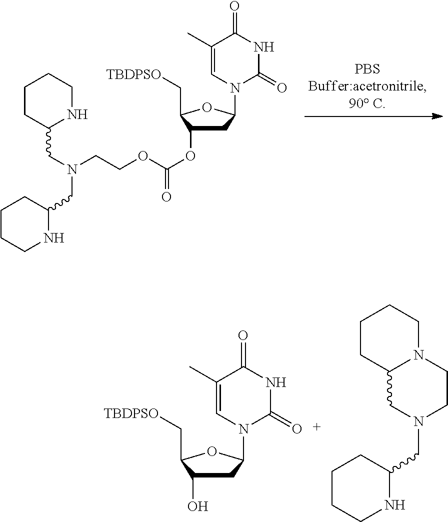

The aim of this study was to investigate whether a linker containing two activating groups (Compound of Example 8C) would have an accelerated linker cleavage time, compared to the linker cleavage of the mono-activating group compound (Compound of Example 1C). The results of this study are shown in FIG. 14. It was found that the extra activating group considerably increased the speed of the linker cleavage. Furthermore the presence of two activating groups (i.e. Ring A) allows greater control of the two steps of the linker cleavage by either carrying out 100% deprotection of both protecting groups, or carrying out the deprotection step only until at least one activating group is deprotected per molecule.

### Example 10—Synthesis of Linkers with Functionality to Attach to a Surface

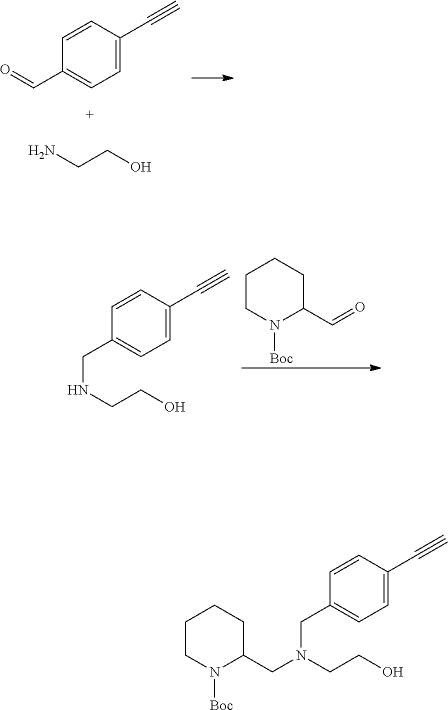

### Example A: 2-((4-Ethynylbenzyl)amino)ethan-1-ol

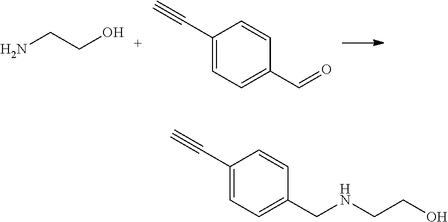

4-Ethynylbenzaldehyde (5 g, 38 mmol) and ethanolamine (2.3 g, 38 mmol) were dissolved in methanol (200 mL), and sodium borohydride (1.4 g, 38 mmol) was added after 10 minutes. The reaction solution was stirred overnight. After this time a water/ethyl acetate workup was carried out, the organic solution was dried (MgSO₄) and the solvent was removed under reduced pressure. The crude product was purified by silica chromatography (0-10% MeOH-DCM) to give a colourless oil which crystallised on standing, 3 g, 45% overall. LC-MS; Method B (Basic); Rt=1.55, m/z 176.1 (MH⁺).

### Example 10B: tert-Butyl 2-(((4-ethynylbenzyl)(2-hydroxyethyl)amino)-methyl)piperidine-1-carboxylate

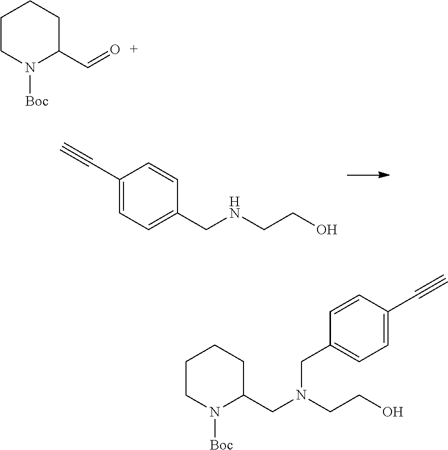 [SMILES: CC(C)(C)OC(=O)N1CCCCC1C=O ; C#Cc1ccc(CNCCO)cc1 ; C#Cc1ccc(CN(CCO)CC2CCCCN2C(=O)OC(C)(C)C)cc1]

2-((4-Ethynylbenzyl)amino)ethan-1-ol (1.6 g, 8.5 mmol) and 1-N-Boc-2-piperidinecarbaldehyde (2 g, 9 mmol, 1.1 eq) were dissolved in 1,2-dichloroethane (80 mL) and acetic acid (3 mL, 85 mmol, 5 eq.) was added and the solution was stirred for 10 minutes at rt. Sodium triacetoxyborohydride was added and after 3 hours there was clean conversion to the product by LC-MS. A DCM/saturated aqueous NaHCO₃ workup was carried out and the organic layer was dried (MgSO₄) and the solvent was removed under reduced pressure to give a pale yellow oil, 3.4 g, 100%. LC-MS; Method A (Acidic); Rt=1.64, m/z 373.3 (MH⁺).

### Example 10C: tert-Butyl 2-(((2-(((((2R,3S,5R)-2-(((tert-butyldiphenylsilyl)oxy)methyl)-5-(5-methyl-2,4-dioxo-3,4-dihydropyrimidin-1(2H)-yl)tetrahydrofuran-3-yl)oxy)carbonyl)oxy)ethyl)(4-ethynylbenzyl)amino)methyl)piperidine-1-carboxylate

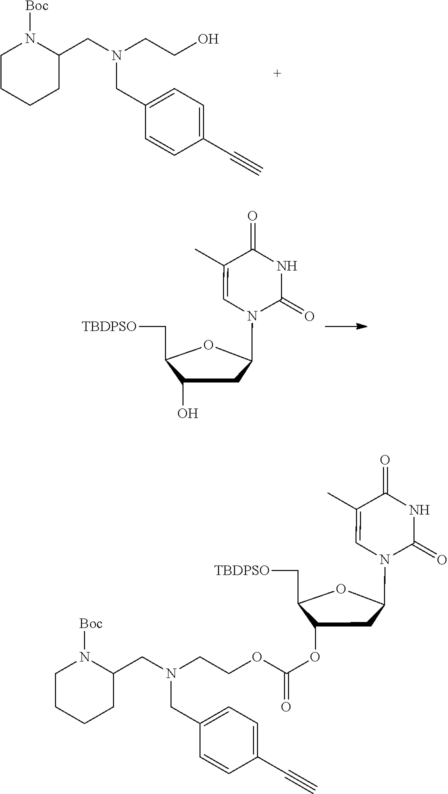

i) 5′-O-TBDPS-Thymidine (2 g, 4.1 mmol) and CDI (797 mg, 4.92 mmol, 1.2 eq.) were dissolved in dry acetonitrile (100 mL) under N₂. The solution was then stirred overnight at rt. After this time there was clean conversion to the active intermediate by LC-MS. tert-Butyl 2-(((4-ethynylbenzyl)(2-hydroxyethyl)amino)methyl)piperidine-1-carboxylate (1.55 g, 4.1 mmol) and DBU (1.2 mL, 8.1 mmol, 2 eq.) were added and the solution was stirred at rt. After 30 minutes the reaction had gone to completion. An ethyl acetate/water workup was carried out and the crude product was purified by silica chromatography (0-100% EtOAc-DCM) to give a pale yellow oil, 2.2 g, 61%. LC-MS; Method A (Acidic); Rt=3.29, m/z 879.6 (MH⁺).

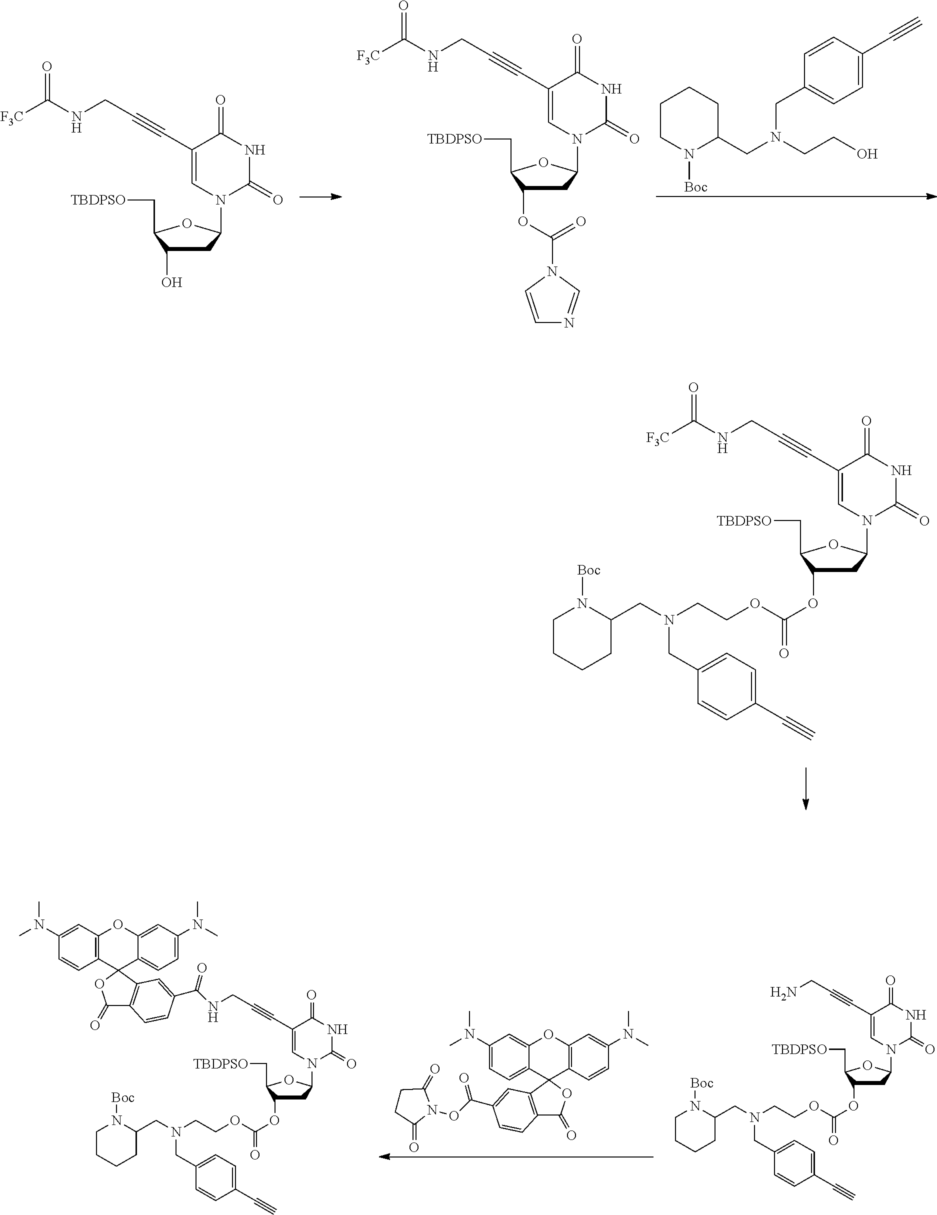

### Example 11

### Example 11A: 1-((2R,4S,5R)-5-(((tert-butyldiphenylsilyl)oxy)methyl)-4-hydroxytetrahydrofuran-2-yl)-5-iodopyrimidine-2,4(1H,3H)-dione

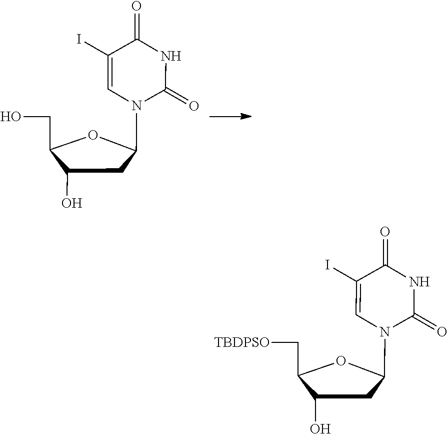

2′-Deoxy-5-iodouridine (5 g, 14 mmol) and imidazole (2.9 g, 42 mmol, 3 eq.) were dissolved in DMF (80 mL) and the solution was cooled in an ice-bath and TBDPSCl (4.2 g, 17 mmol, 1.2 eq.) was added. The solution was warmed to rt and stirred for 2 h after which time the reaction had gone to completion by LC-MS. A water/EtOAc/Brine workup was carried out, the organic layer was dried (MgSO₄) and the solvent was removed to give a pale yellow oil. EtOAc and petrol were added to induce crystallisation and the resulting solid was filtered off with petrol-EtOAc washing to give the product as a white crystalline solid, 5.5 g, 69%. 2016_06_01_012, rt. 2.54, found 593.0, 98% pure. LC-MS; Method A (Acidic); Rt=2.52, m/z 593.0 (MH⁺).

### Example 11B: N-(3-(1-((2R,4S,5R)-5-(((tert-butyldiphenylsilyl)oxy)methyl)-4-hydroxytetrahydrofuran-2-yl)-2,4-dioxo-1,2,3,4-tetrahydropyrimidin-5-yl)prop-2-yn-1-yl)-2,2,2-trifluoroacetamide

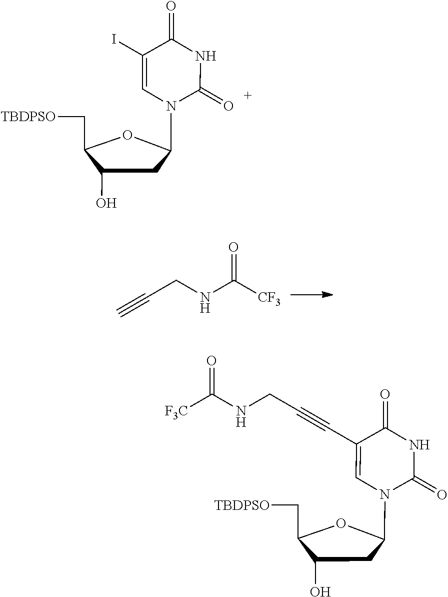

1-((2R,4S,5R)-5-(((tert-butyldiphenylsilyl)oxy)methyl)-4-hydroxytetrahydrofuran-2-yl)-5-iodopyrimidine-2,4(1H,3H)-dione (5 g, 8.4 mmol), 2,2,2-trifluoro-N-(prop-2-yn-1-yl)acetamide (3.8 g, 25.2 mmol, 3 eq.), tetrakis(triphenylphosphine)palladium(0) (1 g, 0.84 mmol, 0.1 eq.), triethylamine (2 mL, 16.8 mmol, 2 eq.) and copper iodide (325 mg, 1.7 mmol, 0.2 eq.) were dissolved in anhydrous DMF (80 mL) under N₂ and the reaction mixture was heated briefly in a hot water bath (40° C.), then stirred at rt for 30 minutes. After this time, the reaction was complete by LC-MS. A water/EtOAc/brine workup was carried out, the organic solution was dried (MgSO₄) and the solvent was removed under reduced pressure. The resulting oil was purified by silica chromatography (0-100% EtOAc-petrol, then 0-5% DCM-Methanol) to give the product as an off-white solid, 3 g, 60%. LC-MS; Method A (Acidic); Rt=2.45, m/z 616.2 (MH⁺).

### Example 11C: tert-Butyl 2-(((2-(((((2R,3S,5R)-2-(((tert-butyldiphenylsilyl)oxy)methyl)-5-(2,4-dioxo-5-(3-(2,2,2-trifluoroacetamido)prop-1-yn-1-yl)-3,4-dihydropyrimidin-1(2H)-yl)tetrahydrofuran-3-yl)oxy)carbonyl)oxy)ethyl)(4-ethynylbenzyl)amino)methyl)piperidine-1-carboxylate

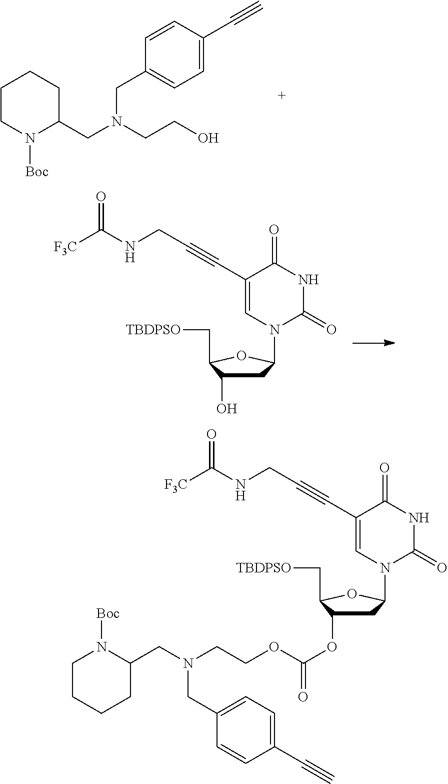

N-(3-(1-((2R,4S,5R)-5-(((tert-Butyldiphenylsilyl)oxy)methyl)-4-hydroxytetrahydrofuran-2-yl)-2,4-dioxo-1,2,3,4-tetrahydropyrimidin-5-yl)prop-2-yn-1-yl)-2,2,2-trifluoroacetamide (2.3 g, 3.7 mmol) and CDI (720 mg, 4.4 mmol, 1.2 eq.) were dissolved in acetonitrile (100 mL) and the solution was stirred under N₂ overnight. After this time tert-butyl 2-(((4-ethynylbenzyl)(2-hydroxyethyl)amino)methyl)piperidine-1-carboxylate (1.4 g, 3.7 mmol) and DBU (1.1 mL, 7.4 mmol, 2 eq.) were added and the solution was stirred at rt. After 30 minutes the reaction had gone to completion by LC-MS. An ethyl acetate/water workup was carried out and the crude product was purified by silica chromatography (0-100% EtOAc-DCM) to give a pale yellow foam, 900 mg, 23%. LC-MS; Method A (Acidic); Rt=3.22, m/z 1014.5 (MH⁺).

### Example 11D: tert-Butyl 2-(((2-(((((2R,3S,5R)-5-(5-(3-aminoprop-1-yn-1-yl)-2,4-dioxo-3,4-dihydropyrimidin-1(2H)-yl)-2-(((tert-butyldiphenylsilyl)oxy)methyl)-tetrahydrofuran-3 yl)oxy)carbonyl)oxy)ethyl)(4-ethynylbenzyl)amino)methyl)piperidine-1-carboxylate

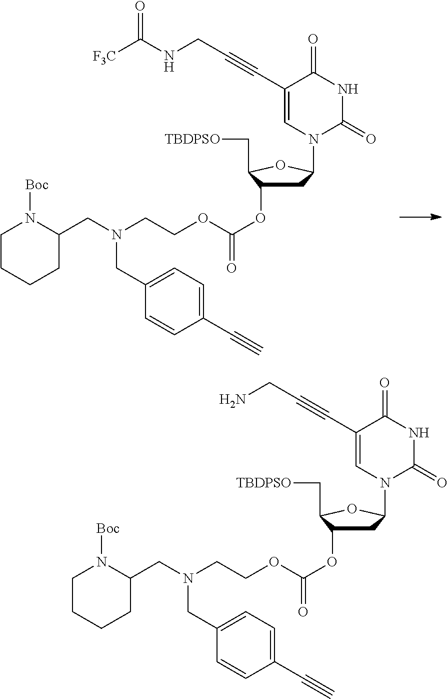

tert-Butyl 2-(((2-(((((2R,3S,5R)-2-(((tert-butyldiphenylsilyl)oxy)methyl)-5-(2,4-dioxo-5-(3-(2,2,2-trifluoroacetamido)prop-1-yn-1-yl)-3,4-dihydropyrimidin-1(2H)-yl)tetrahydrofuran-3-yl)oxy)carbonyl)oxy)ethyl)(4-ethynylbenzyl)amino)methyl)piperidine-1-carboxylate (500 mg, 0.1 mmol) was dissolved in 1:1 25% aqueous ammonia:acetonitrile (20 mL). After 4 h at rt the solvent was removed under reduced pressure and the residue was purified by silica chromatography (0-70% EtOAc-DCM, then 0-10% MeOH-DCM) to give the product as a pale yellow foam, 280 mg, 62%. LC-MS; Method A (Acidic); Rt=2.32, m/z 918.6 (MH⁺).

### Example 11E: tert-Butyl 2-(((2-(((((2R,3S,5R)-5-(5-(3-(3,6′-bis(dimethylamino)-3-oxo-3H-spiro[isobenzofuran-1,9′-xanthene]-6-carboxamido)prop-1 yn-1-yl)-2,4-dioxo-3,4-dihydropyrimidin-1(2H) yl)-2-(((tert-butyldiphenylsilyl)oxy)methyl)tetrahydrofuran-3-yl)oxy)carbonyl)oxy)ethyl)(4-ethynylbenzyl)amino)methyl)piperidine-1-carboxylate

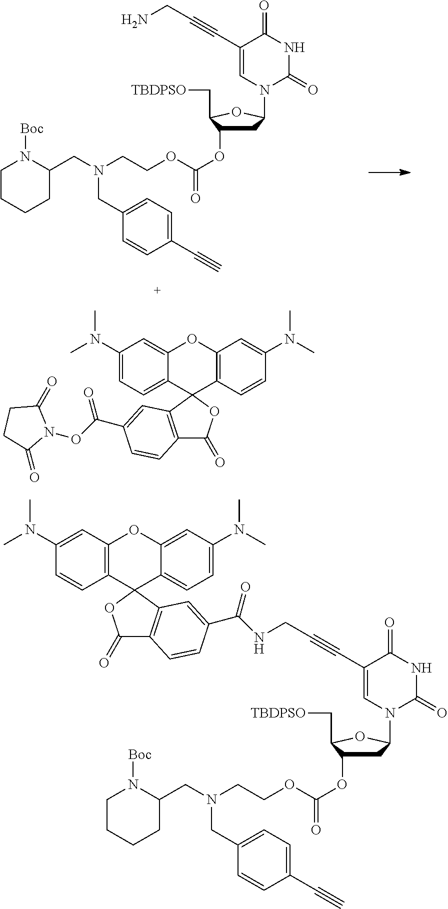

tert-Butyl 2-(((2-(((((2R,3S,5R)-5-(5-(3-aminoprop-1-yn-1-yl)-2,4-dioxo-3,4-dihydro pyrimidin-1(2H)-yl)-2-(((tert-butyldiphenylsilyl)oxy)methyl)tetrahydrofuran-3-yl)oxy)carbonyl)oxy)ethyl)(4-ethynylbenzyl)amino)methyl)piperidine-1-carboxylate (110 mg, 0.12 mmol) was dissolved in DMF (10 mL) and 6-TAMRA N-succinimidyl ester (63 mg, 0.120 mmol) and Hunig's Base (50 μL, 0.24 mmol, 2 eq.) were added. The solution was stirred overnight, after which time the reaction had gone cleanly to completion by LC-MS. The solvent was removed under reduced pressure and the residue was purified by silica chromatography (0-20% Methanol-DCM) to give the product as a purple solid, 146 mg, 91%. LC-MS; Method A (Acidic); Rt=2.57, m/z 666.2 (½ M⁺).

### Example 11F: TAMRA Dye-Tagged 5′-O-TBDPS-Thymidine Attached to Magnetic Beads Via Boc-Protected Linker

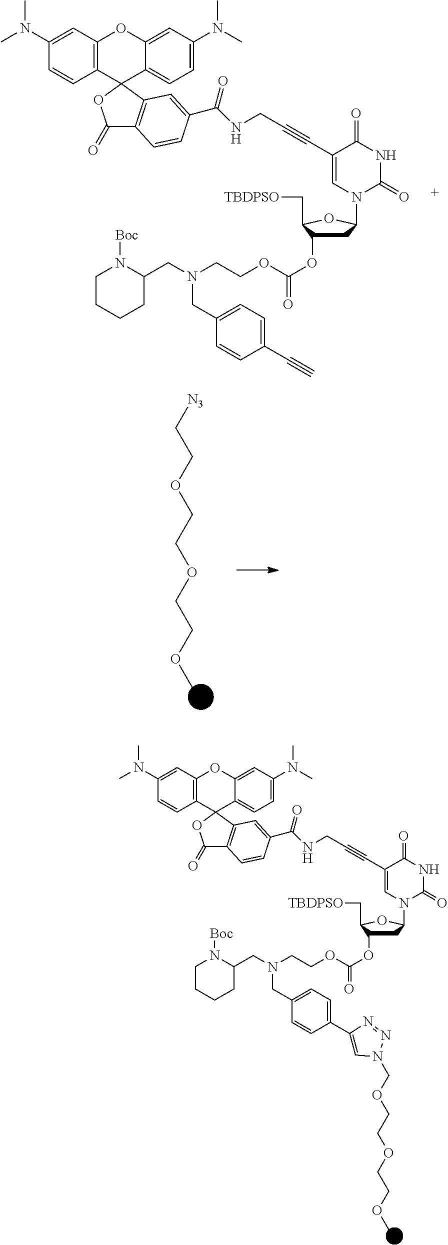

The beads (azide magnetic beads from Kerafast, 1 μm, 30-50 nmol azide groups per mg) were suspended in a solution of tert-Butyl 2-(((2-(((((2R,3S,5R)-5-(5-(3-(3′,6′-bis(dimethylamino)-3-oxo-3H-spiro[isobenzofuran-1,9′-xanthene]-6-carboxamido)prop-1-yn-1-yl)-2,4-dioxo-3,4-dihydropyrimidin-1(2H)-yl)-2-(((tert-butyldiphenylsilyl)oxy)methyl)tetrahydrofuran-3-yl)oxy)carbonyl)oxy)ethyl)(4-ethynylbenzyl)amino)methyl)piperidine-1-carboxylate (6.6 mg, 5 μmol, 10 eq.) in THF (0.5 mL), and a portion was removed to use as a control reaction with no click reagents added. To the main reaction mixture was added aqueous copper sulfate solution (0.1 M, 25 μL, 2.5 μmol) and aqueous sodium ascorbate solution (0.1 M, 50 μL, 5 μmol) and this mixture was stirred vigorously for 3 days. After this time, an identical series of washes were carried out on both sets of beads; 3×THF, 3×DCM, 3×MeOH, 2×THF, 2×MeOH and 2×DCM. Examination with a fluorescent microscope confirmed that the click reaction had successfully taken place in the presence of copper catalyst, but not without copper, and the reacted beads were therefore strongly fluorescent. Furthermore the beads treated with alkyne and catalyst were red, whereas the untreated beads remained brown.

### Example 12—Thermally Mediated Cleavage of TAMRA Dye-Tagged 5′-O-TBDPS-Thymidine from Surface of Beads

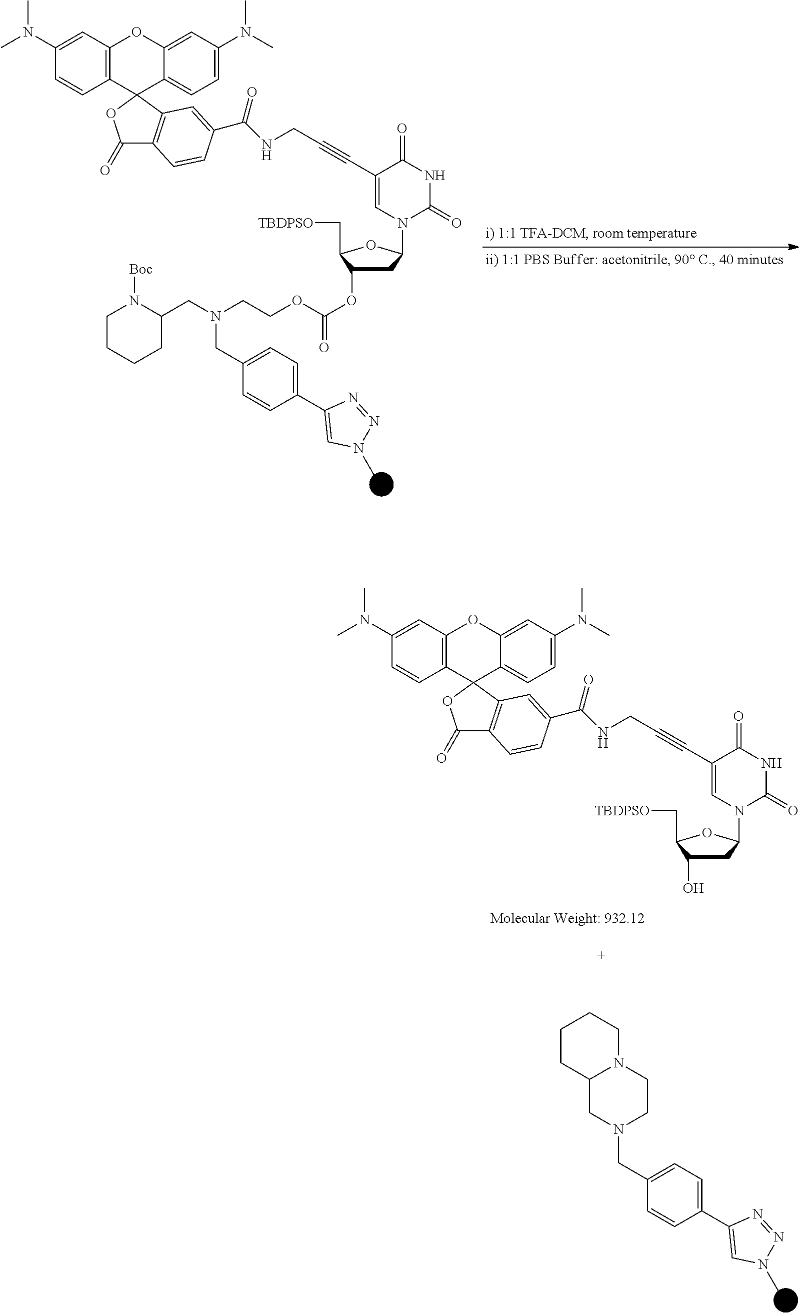

i) The coated beads were stirred vigorously in TFAA:DCM (1:2) at rt for 2 h. After this time, the beads were still red and no cleaved TAMRA-tagged TBPDS-Thymidine was detected in an LC-MS sample of the reaction solution. The beads were then washed with 3×DCM, 3×MeOH, and then 10% Hunig's Base-DCM in order to remove any excess TFAA.

ii) 1 mL of a 1:1 pH 7.4 solution of PBS Buffer and acetonitrile plus 2-3 drops of Hunig's Base was added to the beads and they were heated in a hot-water bath at 90° C. for 40 minutes. After this time, an LC-MS of the reaction solution showed a clear signal for cleaved TAMRA-tagged TBPDS-Thymidine. The beads were washed (3×acetonitrile, 3×MeOH) and examined with a fluorescent microscope, which showed that the fluorescent signal was now much reduced. Furthermore, the beads had reverted to their original brown colour.

### Example 13—Experiments on 5′-Protected Thymidine

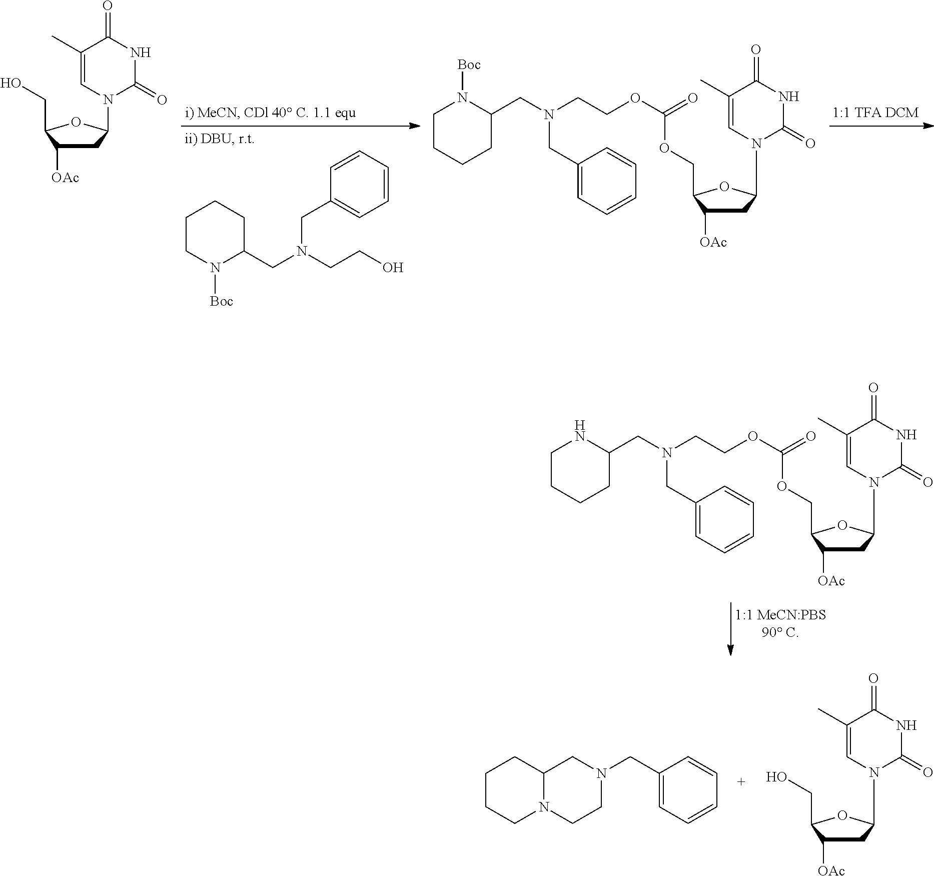

### Example 13A: tert-Butyl 2-(((2-(((((2R,3S,5R)-3-acetoxy-5-(5-methyl-2,4-dioxo-3,4-dihydropyrimidin-1(2H)-yl)tetrahydrofuran-2-yl)methoxy)carbonyl)oxy)ethyl)-(benzyl)amino)methyl)piperidine-1-carboxylate

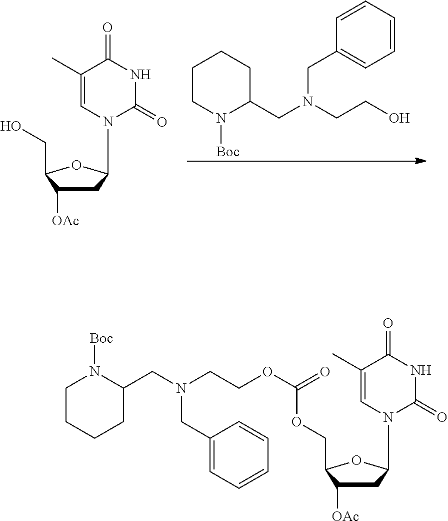

3′-O-Acetyl-thymidine (500 mg, 1.76 mmol, 1.0 eq) and CDI (307 mg, 1.93 mmol, 1.1 eq) were dissolved in anhydrous acetonitrile (40 mL) and the solution was stirred at 40° C. under N₂ for 2 hours. After this time, the reaction was complete by LCMS and was cooled to room temperature. tert-Butyl 2-((benzyl(2-hydroxyethyl)amino)methyl)piperidine-1-carboxylate (673 mg, 1.93 mmol) and DBU (0.28 mL, 1.93 mmol, 1.1 eq.) were then added and the solution was stirred at room temperature for 18 hours. Solvent was removed in vacuo to give a brown oil which was partitioned between water (50 mL) and EtOAc (50 mL) and the layers were separated. The aqueous layer was back extracted with EtOAc (2×50 mL). The combined organic layers was dried (MgSO₄) and the solvent was removed. The resulting oil was purified by silica chromatography, eluting with 0-50% EtOAc-petrol to give the product as a colourless oil, (240 mg, 37%. LC-MS; Method A (Acidic); Rt=1.98, m/z 658.3 (MH⁺).

### Example 13B: (2R,3S,5R)-2-((((2-(benzyl(piperidin-2-ylmethyl)amino)ethoxy)carbonyl)oxy)methyl)-5-(5-methyl-2,4-dioxo-3,4-dihydropyrimidin-1(2H)-yl)tetrahydrofuran-3-yl acetate

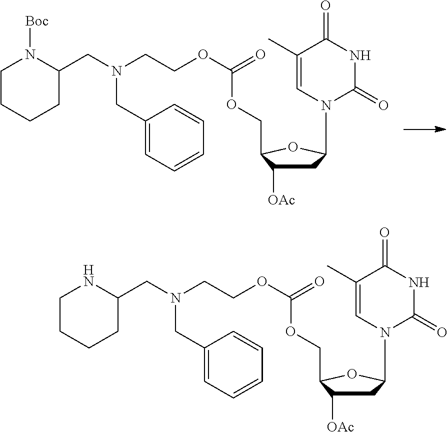

tert-Butyl 2-(((2-(((((2R,3S,5R)-3-acetoxy-5-(5-methyl-2,4-dioxo-3,4-dihydropyrimidin-1(2H)-yl)tetrahydrofuran-2-yl)methoxy)carbonyl)oxy)ethyl)(benzyl)amino)methyl)piperidine-1-carboxylate (100 mg, 0.15 mmol) was dissolved in dichloromethane (2 mL) and trifluoroacetic acid (2 mL), and the solution was stirred at room temperature for 1 h. After this time, the reaction was complete by LC-MS. The solvent was removed and dichloromethane (50 mL) and saturated aqueous NaHCO₃ (50 mL) were added and the layers were separated. The organic layer was dried (MgSO₄) and the solvent was removed under reduced pressure at 20° C. to give a colourless oil which was co evaporated with diethyl ether to give the product as a white solid (40 mg, 47%. LC-MS; Method B (Basic); Rt=1.51, m/z 558 (MH⁺).

### Example 14—Time Course Experiments for Cleavage of the 5′-Protected 3′ O-Acetyl-Thymidine (Compound of Example 13B)

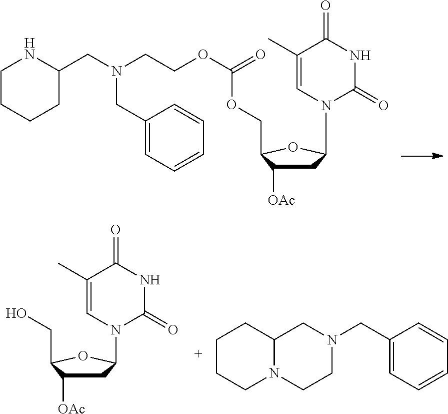

The standard conditions for time course experiments at high and low temperatures were adhered to with the exception of the concentration being 10 mg/ml of (2R,3S,5R)-2-((((2-(benzyl(piperidin-2-ylmethyl)amino)ethoxy)carbonyl)oxy)methyl)-5-(5-methyl-2,4-dioxo-3,4-dihydropyrimidin-1(2H)-yl)tetrahydrofuran-3-yl acetate in MeCN.

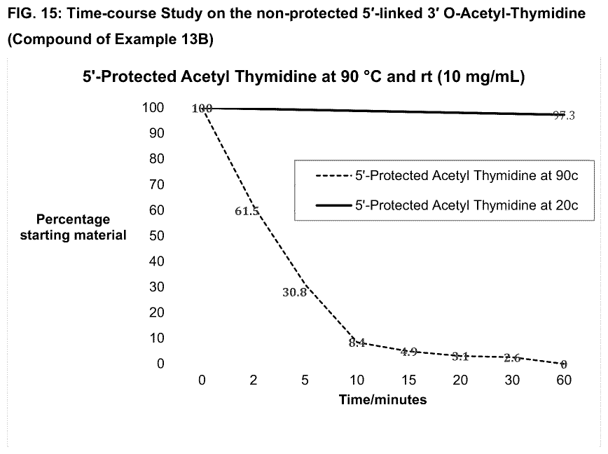

The aim of this study was to demonstrate that the thermal and pH-controlled safety-catch molecule could be used as a protecting group for the 5′-position of a nucleoside. The study showed that the molecule is an effective safety-catch protecting group at this position and would therefore be suitable for use oligonucleotide synthesis. Thus, as shown in FIG. 15, the compound is stable at room temperature, with fast and clean cleavage to release 3′-O-acetyl-thymidine being readily effected at 90° C.

1. A process for the parallel synthesis of one or more oligonucleotides on a plurality of sites on the surface of a solid substrate, said oligonucleotides being the same or different, wherein the process comprises:
Option 1:
(i) providing each site with a plurality of nucleosides, or nucleotides (or wherein the nucleotides are di-nucleotides or tri-nucleotides), wherein each nucleoside or nucleotide comprises a 5′-OH protecting group, and wherein the nucleosides or nucleotides are immobilized on the surface of a solid substrate;
(ii) conducting thermally controlled deprotection at the 5′-OH of the nucleosides or nucleotides, at selected sites on the surface of the solid substrate to form, at each of the selected sites, nucleosides or nucleotides having deprotected 5′—OH groups;
(iii) at each of the selected sites, coupling onto the deprotected 5′—OH groups: a nucleoside 3′-phosphoramidite, or a di-nucleotide 3′-phosphoramidite, or a tri-nucleotide 3′-phosphoramidite, wherein the nucleoside 3′-phosphoramidite, or di-nucleotide 3′-phosphoramidite, or tri-nucleotide 3′-phosphoramidite comprises a 5′-OH protecting group; and oxidising the resulting phosphite triester group to a phosphate triester group;
(iv) conducting thermally controlled deprotection at the 5′-OH of the nucleosides or nucleotides, at selected sites on the surface of the substrate, wherein the selected sites are the same as, or different from, the selected sites of the preceding step,
(v) at each of the selected sites, coupling onto the deprotected 5′—OH groups: a nucleoside 3′-phosphoramidite, or a di-nucleotide 3′-phosphoramidite, or a tri-nucleotide 3′-phosphoramidite, wherein the nucleoside 3′-phosphoramidite, or di-nucleotide 3′-phosphoramidite, or tri-nucleotide 3′-phosphoramidite comprises a 5′-OH protecting group, and oxidising the resulting phosphite triester group to a phosphate triester group; and
(vi) repeating steps (iv) and (v) one or more times to obtain the desired oligonucleotides at each site on the surface of a solid substrate;
wherein the nucleoside or nucleotide comprising a 5′-OH-protecting group in step (iii) and step (v) is:
a nucleoside 3′-phosphoramidite comprising a thermally cleavable 5′-OH protecting group represented by: 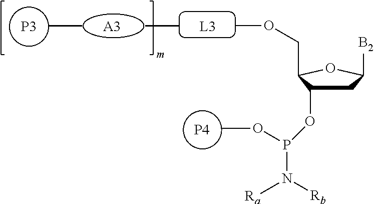
wherein:
P3-A3-L3 together is a safety catch 5′-OH-protecting group, wherein:
P3 is a protecting group
L3 is a cleavable linker moiety,
A3 is an activator moiety that, upon removal of P3, is capable of causing removal of the 5′-OH protecting group;
m is 1 or 2;
P4 is a phosphoramidite protecting group;
B₂ is an optionally protected canonical or an optionally protected non-canonical nucleobase; and
Rₐ and R_{b} are the same or different and each is alkyl; or
a di-nucleotide 3′-phosphoramidite comprising a thermally cleavable 5′-OH protecting group represented by: 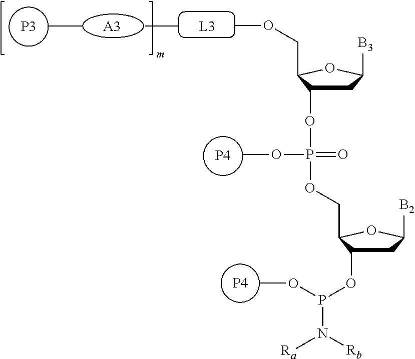
wherein:
P3-A3-L3 together is a safety catch 5′-OH-protecting group, wherein:
P3 is a protecting group
L3 is a cleavable linker moiety,
A3 is an activator moiety that, upon removal of P3, is capable of causing removal of the 5′-OH protecting group;
m is 1 or 2;
each P4 is the same or different and is a phosphoramidite or phosphate protecting group;
B₂ and B₃ are the same or different and each independently is an optionally protected canonical or an optionally protected non-canonical nucleobase; and
Rₐ and R_{b} are the same or different and each is alkyl; or
a tri-nucleotide 3′-phosphoramidite comprising a thermally cleavable 5′-OH protecting group represented by: 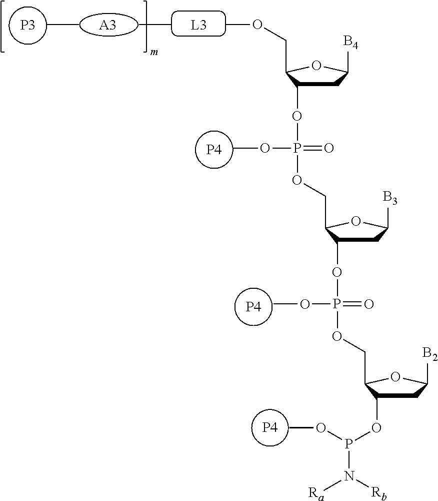
wherein:
P3-A3-L3 together is a safety catch 5′-OH-protecting group, wherein:
P3 is a protecting group
L3 is a cleavable linker moiety,
A3 is an activator moiety that, upon removal of P3, is capable of causing removal of the 5′-OH protecting group;
m is 1 or 2;
each P4 is the same or different and each is a phosphoramidite or phosphate protecting group;
B₂, B₃ and B₄ are the same or different and each independently is an optionally protected canonical or an optionally protected non-canonical nucleobase; and
Rₐ and R_{b} are the same or different and each is alkyl; or
Option 2:
(i) providing each site with a plurality of nucleosides, wherein each nucleoside, comprises a 5′-OH protecting group, and wherein the nucleosides are immobilized on the surface of a solid substrate;
(ii) conducting thermally controlled deprotection at the 5′-OH of the nucleosides at selected sites on the surface of the solid substrate to form, at each of the selected sites, nucleosides having deprotected 5′—OH groups;
(iii) at each of the selected sites, coupling onto the deprotected 5′—OH groups: a nucleoside 3′-phosphoramidite, or a di-nucleotide 3′-phosphoramidite, or a tri-nucleotide 3′-phosphoramidite, wherein the nucleoside 3′-phosphoramidite, or di-nucleotide 3′-phosphoramidite, or tri-nucleotide 3′-phosphoramidite comprises a 5′-OH protecting group; and oxidising the resulting phosphite triester group to a phosphate triester group;
(iv) conducting thermally controlled deprotection at the 5′-OH of the nucleosides at selected sites on the surface of the substrate, wherein the selected sites are the same as, or different from, the selected sites of the preceding step,
(v) at each of the selected sites, coupling onto the deprotected 5′—OH groups: a nucleoside 3′-phosphoramidite, or a di-nucleotide 3′-phosphoramidite, or a tri-nucleotide 3′-phosphoramidite, wherein the nucleoside 3′-phosphoramidite, or di-nucleotide 3′-phosphoramidite, or tri-nucleotide 3′-phosphoramidite comprises a 5′-OH protecting group, and oxidising the resulting phosphite triester group to a phosphate triester group; and
(vi) repeating steps (iv) and (v) one or more times to obtain the desired oligonucleotides at each site on the surface of a solid substrate;
wherein the nucleoside or nucleotide comprising a 5′-OH-protecting group in step (iii) and step (v) is:
a nucleoside 3′-phosphoramidite comprising a thermally cleavable 5′-OH protecting group represented by: 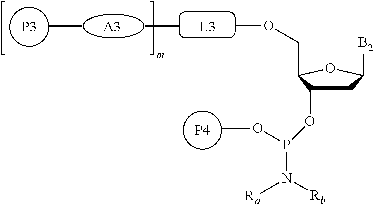
wherein:
P3-A3-L3 together is a safety catch 5′-OH-protecting group, wherein:
P3 is a protecting group
L3 is a cleavable linker moiety,
A3 is an activator moiety that, upon removal of P3, is capable of causing removal of the 5′-OH protecting group;
m is 1 or 2;
P4 is a phosphoramidite protecting group;
B₂ is an optionally protected canonical or an optionally protected non-canonical nucleobase; and
Rₐ and R_{b} are the same or different and each is alkyl; or
a di-nucleotide 3′-phosphoramidite comprising a thermally cleavable 5′-OH protecting group represented by: 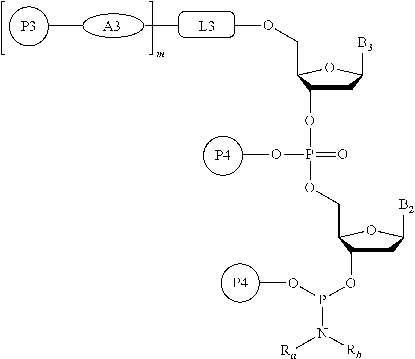
wherein:
P3-A3-L3 together is a safety catch 5′-OH-protecting group, wherein:
P3 is a protecting group
L3 is a cleavable linker moiety,
A3 is an activator moiety that, upon removal of P3, is capable of causing removal of the 5′-OH protecting group;
m is 1 or 2;
each P4 is the same or different and is a phosphoramidite or phosphate protecting group;
B₂ and B₃ are the same or different and each independently is an optionally protected canonical or an optionally protected non-canonical nucleobase; and
Rₐ and R_{b} are the same or different and each is alkyl; or
a tri-nucleotide 3′-phosphoramidite comprising a thermally cleavable 5′-OH protecting group represented by: 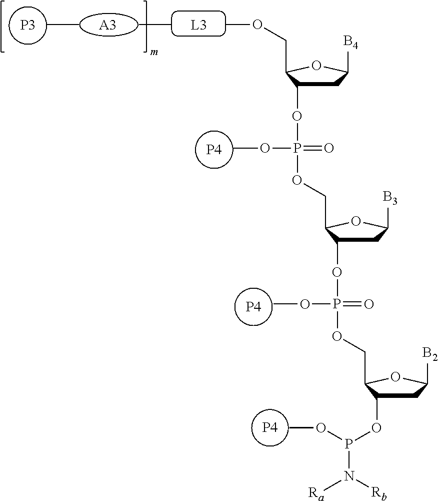
wherein:
P3-A3-L3 together is a safety catch 5′-OH-protecting group, wherein:
P3 is a protecting group
L3 is a cleavable linker moiety,
A3 is an activator moiety that, upon removal of P3, is capable of causing removal of the 5′-OH protecting group;
m is 1 or 2;
each P4 is the same or different and each is a phosphoramidite or phosphate protecting group;
B₂, B₃ and B₄ are the same or different and each independently is an optionally protected canonical or an optionally protected non-canonical nucleobase; and
Rₐ and R_{b} are the same or different and each is alkyl;
or
Option 3:
(i) providing each site with a plurality of nucleosides comprising a 5′-OH protecting group, wherein the nucleosides are immobilized on the surface of a solid substrate;
(ii) conducting thermally controlled deprotection at the 5′-OH of the nucleosides at selected sites on the surface of the solid substrate to form, at each of the selected sites, nucleosides having deprotected 5′—OH groups;
(iii) at each of the selected sites, coupling a nucleoside 3′-phosphoramidite comprising a 5′-OH protecting group, onto the deprotected 5′—OH groups, and oxidising the resulting phosphite triester group to a phosphate triester group;
(iv) conducting thermally controlled deprotection at the 5′-OH of the nucleosides at selected sites on the surface of the substrate, wherein the selected sites are the same as, or different from, the selected sites of the preceding step,
(v) at each of the selected sites, coupling a nucleoside 3′-phosphoramidite comprising a 5′-OH protecting group onto the deprotected 5′—OH groups, and oxidising the resulting phosphite triester group to a phosphate triester group; and
(vi) repeating steps (iv) and (v) one or more times to obtain the desired oligonucleotides at each site on the surface of a solid substrate;
wherein the nucleoside or nucleotide comprising a 5′-OH-protecting group in step (iii) and step (v) is:
a nucleoside 3′-phosphoramidite comprising a thermally cleavable 5′-OH protecting group represented by: 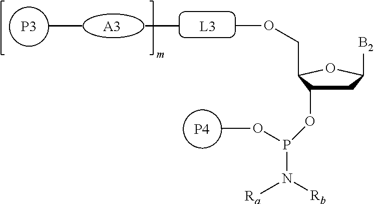
wherein:
P3-A3-L3 together is a safety catch 5′-OH-protecting group, wherein:
P3 is a protecting group
L3 is a cleavable linker moiety,
A3 is an activator moiety that, upon removal of P3, is capable of causing removal of the 5′-OH protecting group;
m is 1 or 2;
P4 is a phosphoramidite protecting group;
B₂ is an optionally protected canonical or an optionally protected non-canonical nucleobase; and
Rₐ and R_{b} are the same or different and each is alkyl; or
a di-nucleotide 3′-phosphoramidite comprising a thermally cleavable 5′-OH protecting group represented by: 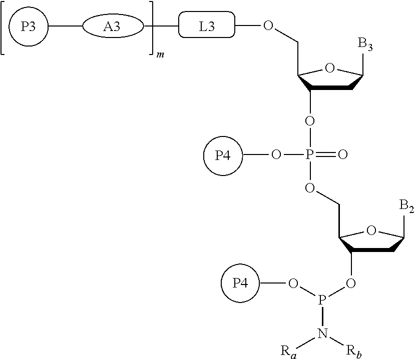
wherein:
P3-A3-L3 together is a safety catch 5′-OH-protecting group, wherein:
P3 is a protecting group
L3 is a cleavable linker moiety,
A3 is an activator moiety that, upon removal of P3, is capable of causing removal of the 5′-OH protecting group;
m is 1 or 2;
each P4 is the same or different and is a phosphoramidite or phosphate protecting group;
B₂ and B₃ are the same or different and each independently is an optionally protected canonical or an optionally protected non-canonical nucleobase; and
Rₐ and R_{b} are the same or different and each is alkyl; or
a tri-nucleotide 3′-phosphoramidite comprising a thermally cleavable 5′-OH protecting group represented by: 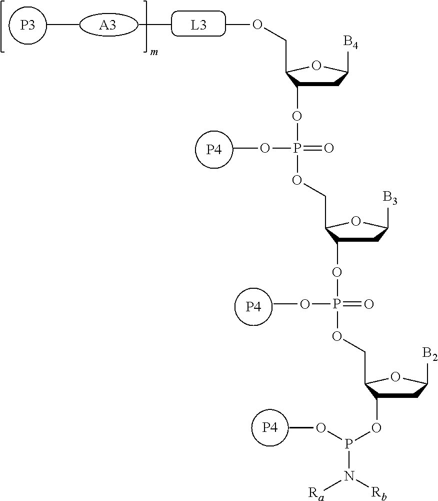
wherein:
P3-A3-L3 together is a safety catch 5′-OH-protecting group, wherein:
P3 is a protecting group
L3 is a cleavable linker moiety,
A3 is an activator moiety that, upon removal of P3, is capable of causing removal of the 5′-OH protecting group;
m is 1 or 2;
each P4 is the same or different and each is a phosphoramidite or phosphate protecting group;
B₂, B₃ and B₄ are the same or different and each independently is an optionally protected canonical or an optionally protected non-canonical nucleobase; and
Rₐ and R_{b} are the same or different and each is alkyl.

2. The process according to claim 1, wherein the nucleosides or nucleotides in step (i) are attached to the surface of a solid substrate at the 3′-position via a thermally cleavable linker group, optionally wherein:
the thermally cleavable linker group comprises one or two activator moieties, and a cleavable linker moiety that on heating, causes the linker group to cleave, thereby causing detachment from the surface of the solid substrate; or
the thermally cleavable linker group comprises a safety catch linker, having one or two activator moieties and a cleavable linker moiety, wherein the activator moiety is protected with a protecting group, wherein the protecting group on each activator moiety is susceptible to deprotection under predetermined conditions, to expose the activator moiety, thereby rendering the activator moiety and cleavable linker moiety susceptible to cleavage on heating.

3. The process according to claim 1, wherein the thermally controlled deprotection in steps (ii) and (iv) is achieved by local heating at the selected sites; optionally wherein there is substantially no deprotection of the 5′-OH protecting groups at sites other than the selected sites.

4. The process according to claim 1, wherein the coupling steps (iii) and (v) comprise contacting a solution containing the nucleoside 3′-phosphoramidite, or di-nucleotide 3′-phosphoramidite, or tri-nucleotide 3′-phosphoramidite comprising a 5′-OH protecting group with the surface of the substrate, wherein the nucleoside 3′-phosphoramidite, or di-nucleotide 3′-phosphoramidite, or tri-nucleotide 3′-phosphoramidite reacts with the deprotected 5′—OH groups at the selected sites; optionally wherein there is substantially no reaction at the sites other than the selected sites.

5. The process according to claim 1, wherein step (i) comprises providing at each site: a plurality of nucleosides or nucleotides immobilized to the solid surface, represented by: 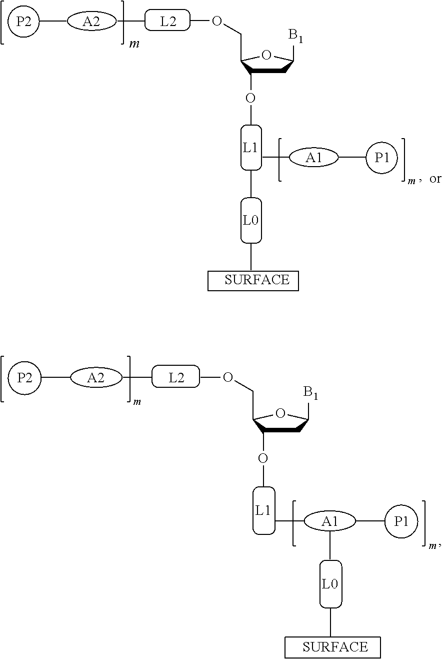
wherein:
L1-A1-P1 together is a safety catch linker for attachment at the 3′—OH group of the nucleoside to the surface, wherein:
P1 is a protecting group,
L1 is a thermally cleavable linker moiety,
A1 is an activator moiety that, upon removal of P1, is capable of causing cleavage of the cleavable linker from the solid surface;
P2-A2-L2 together is a safety catch 5′-OH-protecting group, wherein:
P2 is a protecting group,
L2 is a cleavable linker moiety,
A2 is an activator moiety that, upon removal of P2, is capable of causing removal of the 5′-OH protecting group;
m at each occurrence is the same or different, and is 1 or 2;
L0 is a moiety for attachment of the first nucleoside via the cleavable linker group to the surface; and
B₁ is an optionally protected canonical or an optionally protected non-canonical nucleobase,
wherein A1, A2, L1 and L2 are the same or different, and wherein P1 and P2 are different and are removable under different conditions or reagents; or 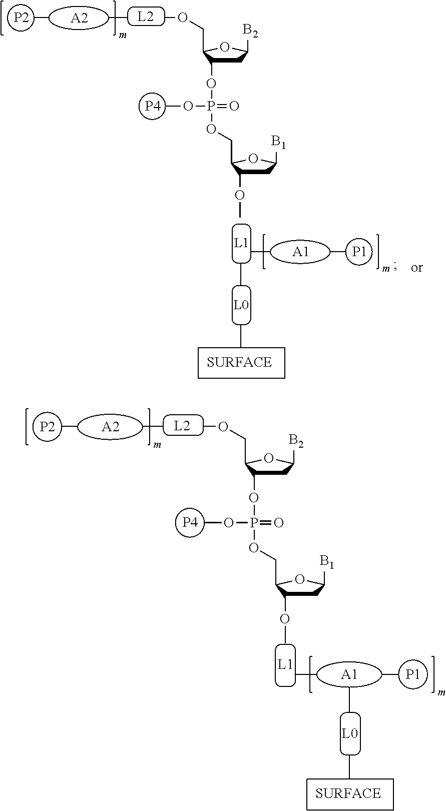
wherein:
L1-A1-P1 together is a safety catch linker for attachment at the 3′—OH group of the nucleotide to the surface, wherein:
P1 is a protecting group,
L1 is a thermally cleavable linker moiety,
A1 is an activator moiety that, upon removal of P1, is capable of causing cleavage of the cleavable linker from the solid surface;
P2-A2-L2 together is a safety catch 5′-OH-protecting group, wherein:
P2 is a protecting group,
L2 is a cleavable linker moiety,
A2 is an activator moiety that, upon removal of P2, is capable of causing removal of the 5′-OH protecting group;
P4 is a phosphate protecting group,
m at each occurrence is the same or different, and is 1 or 2;
L0 is a moiety for attachment of the first nucleoside via the cleavable linker group to the surface; and
B₁ and B₂ are the same or different and each independently is an optionally protected canonical or an optionally protected non-canonical nucleobase,
wherein A1, A2, L1 and L2 are the same or different, and wherein P1 and P2 are different and are removable under different conditions or reagents; or 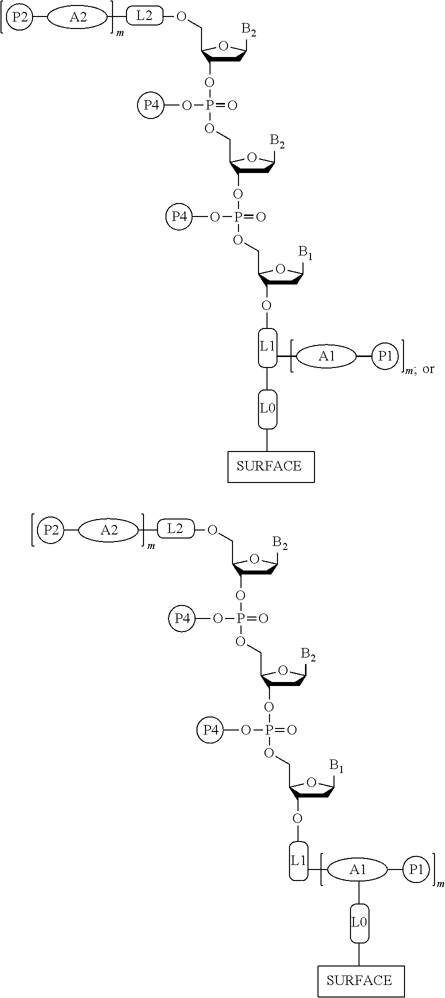
wherein:
L1-A1-P1 together is a safety catch linker for attachment at the 3′—OH group of the nucleotide to the surface, wherein:
P1 is a protecting group,
L1 is a thermally cleavable linker moiety,
A1 is an activator moiety that, upon removal of P1, is capable of causing cleavage of the cleavable linker from the solid surface;
P2-A2-L2 together is a safety catch 5′-OH-protecting group, wherein:
P2 is a protecting group,
L2 is a cleavable linker moiety,
A2 is an activator moiety that, upon removal of P2, is capable of causing removal of the 5′-OH protecting group;
each P4 is the same or different and each independently is a phosphate protecting group,
m at each occurrence is the same or different, and is 1 or 2;
L0 is a moiety for attachment of the first nucleoside via the cleavable linker group to the surface; and
B₁, B₂ and B₃ are the same or different and each independently is an optionally protected canonical or an optionally protected non-canonical nucleobase,
wherein A1, A2, L1 and L2 are the same or different, and wherein P1 and P2 are different and are removable under different conditions or reagents;
optionally wherein step (i) comprises providing at each site, a plurality of nucleosides immobilized to the solid surface, represented by: 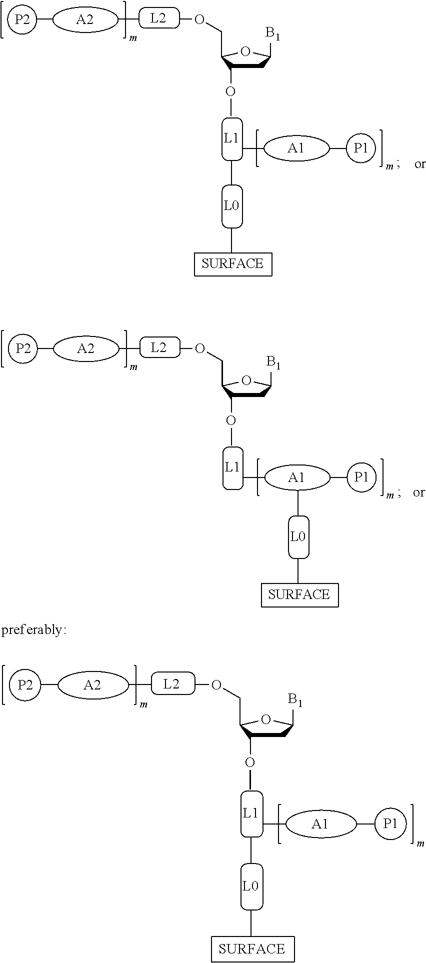
wherein:
L1-A1-P1 together is a safety catch linker for attachment at the 3′—OH group of the nucleoside to the surface, wherein:
P1 is a protecting group,
L1 is a thermally cleavable linker moiety,
A1 is an activator moiety that, upon removal of P1, is capable of causing cleavage of the cleavable linker from the solid surface;
P2-A2-L2 together is a safety catch 5′-OH-protecting group, wherein:
P2 is a protecting group,
L2 is a cleavable linker moiety,
A2 is an activator moiety that, upon removal of P2, is capable of causing removal of the 5′-OH protecting group;
m at each occurrence is the same or different, and is 1 or 2;
L0 is a moiety for attachment of the first nucleoside via the cleavable linker group to the surface; and
B₁ is an optionally protected canonical or an optionally protected non-canonical nucleobase.

6. The process according to claim 5, wherein step (ii) comprises thermally controlled removal of the safety catch 5′-OH protecting cleavable linker group P2-A2-L2.

7. The process according to claim 1, wherein the coupling of: the nucleoside 3′-phosphoramidite comprising a 5′-OH protecting group, or the di-nucleotide 3′-phosphoramidite comprising a 5′-OH protecting group, or the tri-nucleotide 3′-phosphoramidite comprising a 5′-OH protecting group, in step (iii) to a deprotected 5′—OH group of the immobilized nucleoside or nucleotide, followed by oxidation, forms a structure represented by: 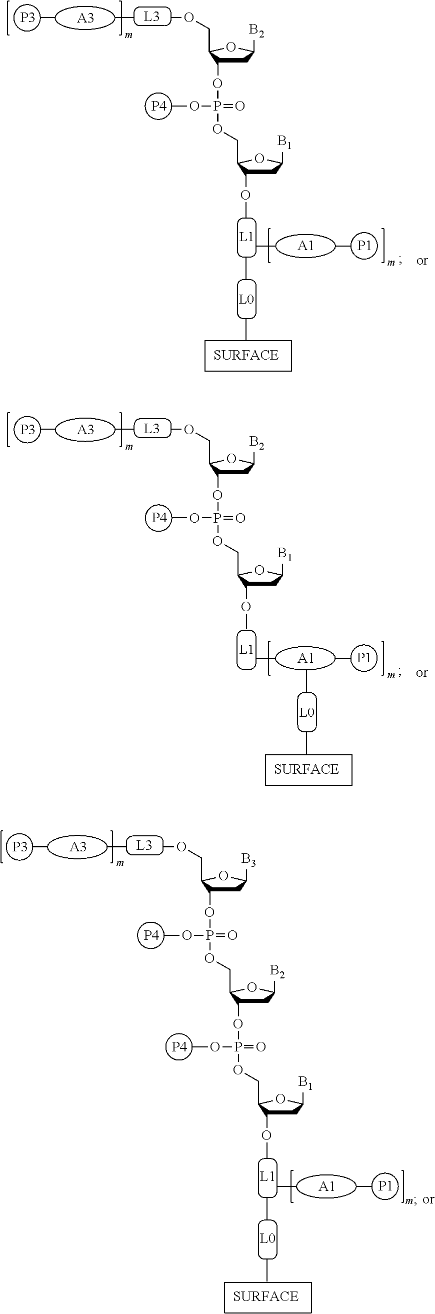 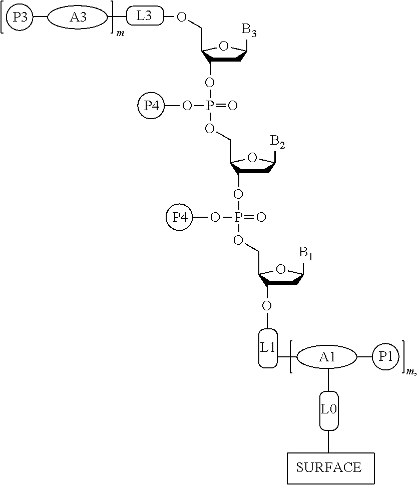
wherein:
L1-A1-P1 together is a safety catch linker for attachment at the 3′—OH group of the nucleoside to the surface, wherein:
P1 is a protecting group,
L1 is a thermally cleavable linker moiety,
A1 is an activator moiety that, upon removal of P1, is capable of causing cleavage of the cleavable linker from the solid surface;
P3-A3-L3 together is a safety catch 5′-OH protecting group, wherein:
P3 is a protecting group,
L3 is a cleavable linker moiety,
A3 is an activator moiety that, upon removal of P3, is capable of causing removal of the 5′-OH protecting group;
m at each occurrence is the same or different, and is 1 or 2;
L0 is a moiety for attachment of the first nucleoside via the cleavable linker group to the surface;
each B₁, or B₂ or B₃ independently is an optionally protected canonical or an optionally protected non-canonical nucleobase,
wherein A1, A3, L1 and L3 are the same or different, and wherein P1 and P3 are different and are removable under different conditions or reagents; and
each P4 is the same or different and each is a phosphate protecting group.

8. The process according to claim 1, wherein steps (ii) and (iii) are repeated, to sequentially grow the oligonucleotides at each site by successive thermally controlled deprotection at the 5′-OH of the nucleosides or nucleotides and coupling of an incoming nucleoside or nucleotide represented by: 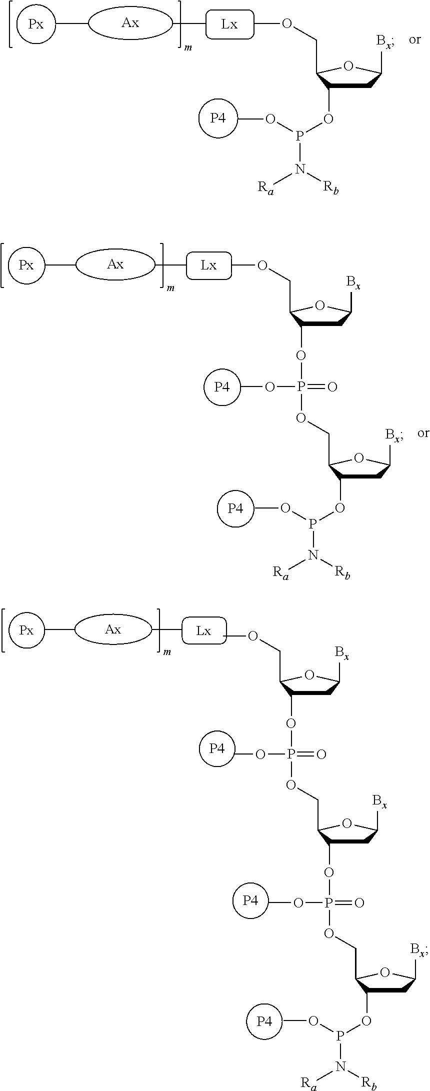
wherein:
Px-Ax-Lx together is a cleavable 5′-OH protecting group which protects the 5′—OH group of the incoming nucleoside or nucleotide, wherein:
Lx is a cleavable linker moiety,
Px is a protecting group, and
Ax is an activator moiety that, upon removal of Px, is capable of causing removal of the 5′-OH protecting group;
m is 1 or 2;
each P4 is the same or different and each is a phosphoramidite or phosphate protecting group;
each Bₓ is the same or different and each independently is an optionally protected canonical or an optionally protected non-canonical nucleobase; and
Rₐ and R_{b} are the same or different and each is alkyl.

9. The process according to claim 1, wherein the 5′-OH-protected nucleosides of step (i) comprise a thermally cleavable 5′-OH-protecting group, and are attached to the surface of a solid substrate at the 3′-position via a thermally cleavable linker group, wherein the thermally cleavable linker attaching the first nucleoside to the surface is stable to removal during the oligonucleotide synthesis steps.

10. The process according to claim 9, wherein the protecting groups on the nucleobases, when present, are stable to removal during the oligonucleotide synthesis; the protecting groups P4 on the nucleoside 3′-phosphoramidites, or the di-nucleotide 3′-phosphoramidites, or the tri-nucleotide 3′-phosphoramidites, are stable to removal during the oligonucleotide synthesis; or combinations thereof.

11. The process according to claim 1, wherein step (i) comprises:
(a) providing a solid surface comprising a plurality of sites, wherein each site is functionalized with thermally labile linker groups, each of which is represented by: 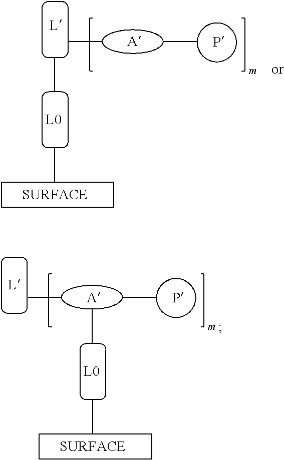
wherein:
L′-A′-P′ together is a safety catch linker attached to the surface via L0, wherein:
P′ is a protecting group,
L′ is a cleavable linker moiety,
A′ is an activator moiety that, upon removal of P′, is capable of causing cleavage of the cleavable linker group from the solid surface;
m is 1 or 2;
L0 is a moiety for attachment of the cleavable linker group to the surface;
(b) removing the protecting group P′, thereby resulting in a solid surface comprising a plurality of sites represented by: 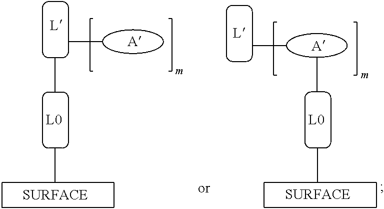
(c) thermally controlled deprotection of the cleavable linker L′ via activator moiety A′ at selected sites and coupling the deprotected sites with:
a nucleoside represented by the formula: 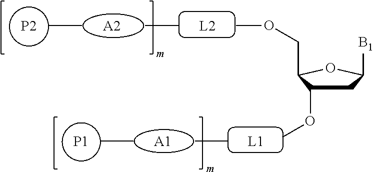
wherein:
L1-A1-P1 together is a safety catch linker for attachment at the 3′—OH group of the first nucleoside to the surface, wherein:
P1 is a protecting group,
L1 is a cleavable linker moiety,
A1 is an activator moiety that, upon removal of P1, is capable of causing cleavage of the cleavable linker from the solid surface;
P2-A2-L2 together is a safety catch 5′-OH protecting group, wherein:
P2 is a protecting group,
L2 is a cleavable linker moiety,
A2 is an activator moiety that, upon removal of P2, is capable of causing removal of the 5′-OH protecting group;
m at each occurrence is the same or different, and is 1 or 2; and
B₁ is an optionally protected canonical or an optionally protected non-canonical nucleobase, optionally wherein the nucleobase is one of: adenine (A), cytosine (C), guanine (G) or thymine (T); or
a di-nucleotide represented by the formula: 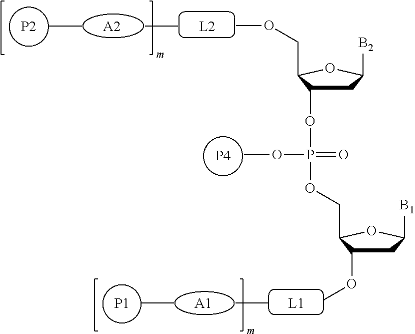
wherein:
L1-A1-P1 together is a safety catch linker for attachment at the 3′—OH group of the first nucleoside to the surface, wherein:
P1 is a protecting group,
L1 is a cleavable linker moiety,
A1 is an activator moiety that, upon removal of P1, is capable of causing cleavage of the cleavable linker from the solid surface;
P2-A2-L2 together is a safety catch 5′-OH protecting group, wherein:
P2 is a protecting group,
L2 is a cleavable linker moiety,
A2 is an activator moiety that, upon removal of P2, is capable of causing removal of the 5′-OH protecting group;
P4 is a phosphate protecting group;
m at each occurrence is the same or different, and is 1 or 2; and
B₁ and B₂ are the same or different, and each independently is an optionally protected canonical or an optionally protected non-canonical nucleobase, optionally wherein the nucleobase is one of: adenine (A), cytosine (C), guanine (G) or thymine (T); or
a tri-nucleotide represented by the formula: 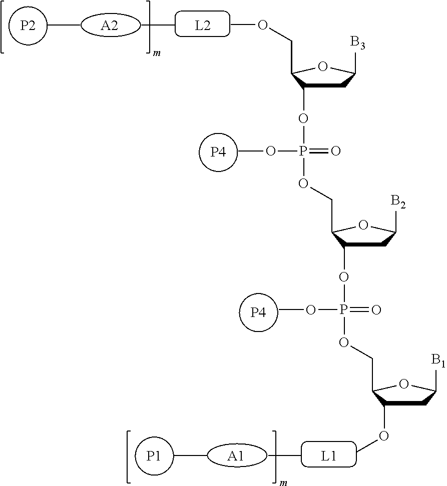
wherein:
L1-A1-P1 together is a safety catch linker for attachment at the 3′—OH group of the first nucleoside to the surface, wherein:
P1 is a protecting group,
L1 is a cleavable linker moiety,
A1 is an activator moiety that, upon removal of P1, is capable of causing cleavage of the cleavable linker from the solid surface;
P2-A2-L2 together is a safety catch 5′-OH protecting group, wherein:
P2 is a protecting group,
L2 is a cleavable linker moiety,
A2 is an activator moiety that, upon removal of P2, is capable of causing removal of the 5′-OH protecting group;
each P4 is the same or different, and each independently is a phosphate protecting group;
m at each occurrence is the same or different, and is 1 or 2; and
B₁, B₂ and B₃ are the same or different, and each independently is an optionally protected canonical or an optionally protected non-canonical nucleobase, optionally wherein the nucleobase is one of: adenine (A), cytosine (C), guanine (G) or thymine (T);
(d) thermally controlled deprotection of the cleavable linker L′ via activator moiety A′ at selected sites which were not deprotected in the preceding step, and coupling the deprotected sites with another nucleoside, optionally a nucleoside comprising one of the other three canonical nucleobases; and
(e) repeating step (d) with the other remaining nucleosides;
thereby forming a plurality of sites on a solid surface wherein the solid surface comprises a plurality of 5′-OH-protected nucleosides or nucleotides containing nucleobases, wherein the nucleobases are optionally protected canonical or an optionally protected non-canonical nucleobase, and optionally wherein the nucleobases are A, C, G and T, and wherein the nucleosides are each attached at the 3′-OH to the solid surface via a cleavable linker group L1-A1-P1.

12. The process according to claim 1, wherein step (i) comprises:
(a) providing a solid surface comprising a plurality of sites, wherein each site is functionalized with thermally labile linker groups, each of which is represented by: 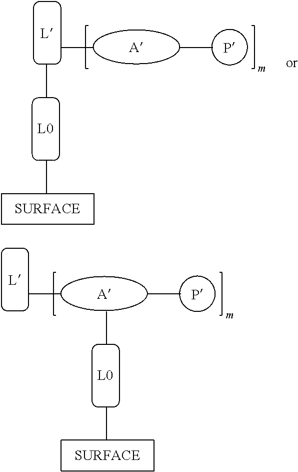
wherein:
L′-A′-P′ together is a safety catch linker attached to the surface via L0, wherein:
P′ is a protecting group,
L′ is a cleavable linker moiety,
A′ is an activator moiety that, upon removal of P′, is capable of causing cleavage of the cleavable linker group from the solid surface;
m is 1 or 2;
L0 is a moiety for attachment of the cleavable linker group to the surface;
(b) removing the protecting group P′, thereby resulting in a solid surface comprising a plurality of sites represented by: 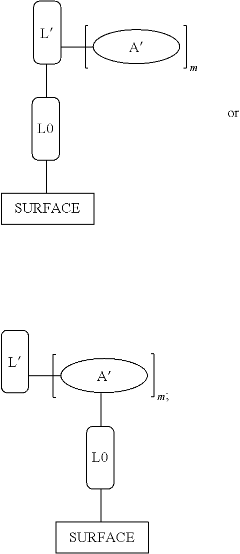
(c) thermally controlled deprotection of the cleavable linker L′ via activator moiety A′ at selected sites and coupling the deprotected sites with a nucleoside represented by the formula: 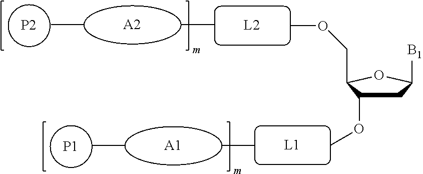
wherein:
L1-A1-P1 together is a safety catch linker for attachment at the 3′—OH group of the first nucleoside to the surface, wherein:
P1 is a protecting group,
L1 is a cleavable linker moiety,
A1 is an activator moiety that, upon removal of P1, is capable of causing cleavage of the cleavable linker from the solid surface;
P2-A2-L2 together is a safety catch 5′-OH protecting group, wherein:
P2 is a protecting group,
L2 is a cleavable linker moiety,
A2 is an activator moiety that, upon removal of P2, is capable of causing removal of the 5′-OH protecting group;
m at each occurrence is the same or different, and is 1 or 2; and
B₁ is an optionally protected canonical or an optionally protected non-canonical nucleobase, optionally wherein the nucleobase is one of: adenine (A), cytosine (C), guanine (G) or thymine (T),
(d) thermally controlled deprotection of the cleavable linker L′ via activator moiety A′ at selected sites which were not deprotected in the preceding step, and coupling the deprotected sites with another nucleoside, optionally a nucleoside comprising one of the other three canonical nucleobases; and
(e) repeating step (d) with the other remaining nucleosides;
thereby forming a plurality of sites on a solid surface wherein the solid surface comprises a plurality of 5′-OH-protected nucleosides containing nucleobases, wherein the nucleobases are optionally protected canonical or an optionally protected non-canonical nucleobase, and optionally wherein the nucleobases are A, C, G and T, and wherein the nucleosides are each attached at the 3′-OH to the solid surface via a cleavable linker group L1-A1-P1.

13. The process according to claim 1, wherein the thermal control of the deprotection of the oligonucleotide is provided by individually thermally addressable sites on a chip.

14. The process according to claim 1, for the parallel synthesis of one or more oligonucleotides on a plurality of sites on the surface of solid substrate, wherein the solid substrate comprises a chip, said oligonucleotides being the same or different wherein the process comprises:
(i) providing each site with a plurality of nucleosides comprising a 5′-OH thermally cleavable protecting group, wherein the nucleosides are attached to the surface of a solid substrate at the 3′-position via a thermally cleavable linker group:
(ii) thermally controlled deprotection at the 5′-OH of the nucleosides at selected sites on the surface of the chip to form, at each of the selected sites, nucleosides having deprotected 5′—OH groups;
(iii) at each of the selected sites, coupling onto the deprotected 5′—OH groups a nucleoside 3′-phosphoramidite, a di-nucleotide 3′-phosphoramidite, or a tri-nucleotide 3′-phosphoramidite, wherein the nucleoside 3′-phosphoramidite, di-nucleotide 3′-phosphoramidite, or tri-nucleotide 3′-phosphoramidite comprises a thermally cleavable 5′-OH protecting group; and oxidising the resulting phosphite triester group to a phosphate triester group;
(iv) thermally controlled deprotection at the 5′-OH of the nucleosides at selected sites on the surface of the substrate, wherein the selected sites are the same as, or different from, the selected sites of the preceding step,
(v) at each of the selected sites, coupling onto the deprotected 5′—OH groups, a nucleoside 3′-phosphoramidite, a di-nucleotide 3′-phosphoramidite, or a tri-nucleotide 3′-phosphoramidite, wherein the nucleoside 3′-phosphoramidite, di-nucleotide 3′-phosphoramidite, or tri-nucleotide 3′-phosphoramidite comprises a thermally cleavable 5′-OH protecting group; and oxidising the resulting phosphite triester group to a phosphate triester group; and
(vi) repeating steps (iv) and (v) one or more times to obtain the desired oligonucleotides at each site on the surface of the chip, wherein the chip comprises individually thermally addressable sites; optionally wherein step (v) comprises:
(v) at each of the selected sites, coupling a nucleoside 3′-phosphoramidite comprising a thermally cleavable 5′-OH protecting group, onto the deprotected 5′—OH groups, and oxidising the resulting phosphite triester group to a phosphate triester group.

15. The process according to claim 1, wherein the solid substrate comprises a temperature control device for controlling temperatures at a plurality of sites of the solid substrate, comprising:
(i) a plurality of active thermal sites disposed at respective locations on the substrate, each active thermal site comprising a heating element configured to apply a variable amount of heat to a corresponding site of said medium and a thermal insulation layer disposed between the heating element and the substrate; and
(ii) one or more passive thermal regions disposed between the plurality of active thermal sites on the substrate, each passive thermal region comprising a thermal conduction layer configured to conduct heat from a corresponding portion of the medium to the substrate;
wherein the thermal conduction layer of said one or more passive thermal regions has a lower thermal resistance in a direction perpendicular to a plane of the substrate than the thermal insulation layer of said plurality of active thermal sites.

16. The process according to claim 2, wherein the thermally cleavable linker group is represented by the formula (L-1): 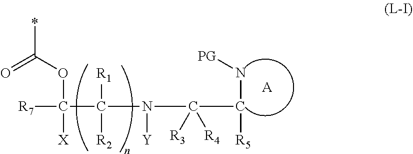
wherein:
* is a point of attachment to the 3′-OH of the nucleoside;
X is hydrogen or hydrocarbyl;
Y is hydrocarbyl or 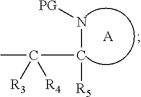
each of R₁, R₂, R₃, R₄, R₅ and R₇ are the same or different and each independently is hydrogen or hydrocarbyl;
PG is a cleavable protecting group for nitrogen;
n is 0, 1, 2 or 3; and
ring A is a nitrogen-containing heterocyclic group;
wherein at each occurrence R₁, R₂, R₃, R₄, R₅, PG and A are the same or different, which is bound to the substrate at one of R₁, R₂, R₃, R₄, R₅, R₇, X, Y or A, optionally which is bound to the substrate at R₇ or Y, or optionally which is bound to the substrate at R₇ when Y is: 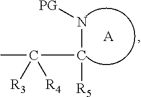
or wherein the cleavable linker is bound to the substrate at Y when Y is hydrocarbyl;
optionally wherein:
(a) at least one of the protecting groups PG is cleavable under a first reaction condition to produce a deprotected linker, wherein the deprotected linker can undergo intramolecular cyclisation and cleavage with release of carbon dioxide under a second, different, reaction condition, to produce a compound of formula (II): 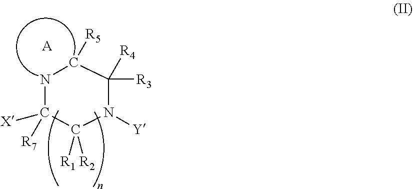
thereby releasing the oligonucleotide from the surface;
wherein PG′ is hydrogen or a cleavable protecting group for nitrogen, provided that at least one PG′ is hydrogen;
Y′ is hydrocarbyl, or 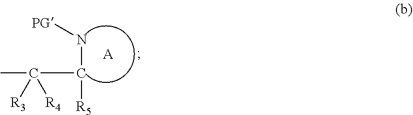
Y is hydrocarbyl, optionally wherein Y is a C₁₋₂₀ hydrocarbyl or a C₁₋₁₀ or particularly a C₁₋₆ hydrocarbyl, or optionally wherein the C₁₋₂₀ or C₁₋₁₀ or C₁₋₆ hydrocarbyl is alkyl, aryl, alkaryl and arylalkyl, alkenyl, or alkynyl, or optionally wherein Y is C₁₋₁₀ alkyl or C₆₋₁₀ aryl; or
(c) combinations of (a) and (b).

17. The process according to claim 1, wherein the 5′-OH protecting group is represented by the formula (L-1′): 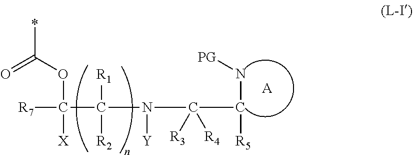
wherein:
* is a point of attachment to the 5′-OH of the nucleoside;
X is hydrogen or hydrocarbyl;
Y is hydrocarbyl or 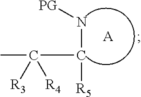
each of R₁, R₂, R₃, R₄, R₅ and R₇ are the same or different and each independently is hydrogen or hydrocarbyl;
PG is a cleavable protecting group for nitrogen which is different from the PG group in formula L-1;
n is 0, 1, 2 or 3; and
ring A is nitrogen-containing heterocyclic group;
wherein at each occurrence R₁, R₂, R₃, R₄, R₅, PG and A are the same or different;
optionally wherein at least one of the protecting groups PG is cleavable under a first reaction condition to produce a deprotected linker, wherein the deprotected linker can undergo intramolecular cyclisation and cleavage with release of carbon dioxide under a second, different, reaction condition, to produce a compound of formula (II): 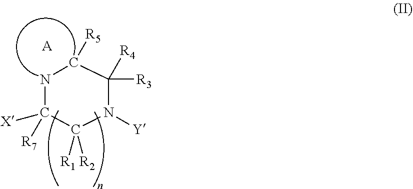
thereby deprotecting the 5′—OH group of the nucleoside;
wherein
PG′ is hydrogen or a cleavable protecting group for nitrogen, provided that at least one PG′ is hydrogen;
Y′ is hydrocarbyl, or

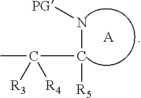

18. The process according to claim 17, wherein the 5′-OH protecting group has the formula (IB′): 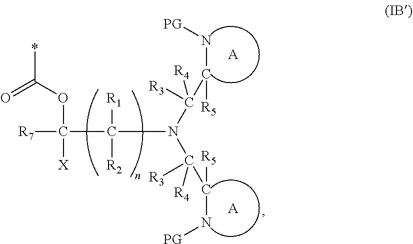
wherein R₁-R₅, PG and A at each occurrence in formula (IB), is the same;
and/or wherein at least one of the protecting groups PG is cleavable under a first reaction condition to produce a compound of formula (IB*′): 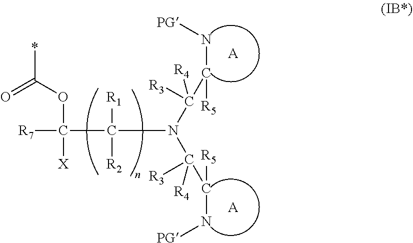
wherein
PG′ is hydrogen or a cleavable protecting group for nitrogen, provided that at least one PG′ is hydrogen;
wherein the compound of Formula (IB*) can undergo intramolecular cyclisation and cleavage with release of carbon dioxide under a second, different reaction condition, to produce a compound of formula (IIB′): 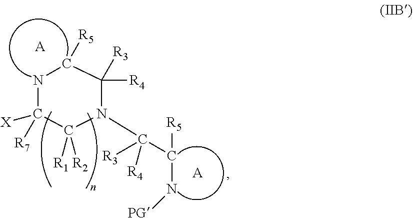
thereby removing the protecting group at 5′-OH.

19. The process according to claim 16, wherein ring A is a 4-12 membered mono-, bi- or tri-cyclic, optionally mono- or bicyclic nitrogen-containing heterocyclic group, and which may contain, in addition to the nitrogen, one or more other heteroatoms that are N, O or S, optionally O or N; optionally wherein: ring A is a 4 to 8-membered monocyclic heterocyclic group; or
ring A is a 5, 6, or 7-membered monocyclic heterocyclic group; or
ring A is a heterocycle that is piperidyl, morpholinyl, pyrrolidinyl, thiomorpholinyl, or imidazolyl; or
ring A is piperidyl, pyrrolidinyl or imidazolyl; or
ring A is piperidyl, or pyrrolidinyl.

20. The process according to claim 16, wherein at each occurrence of —C(R₃)(R₄), one of R₃ or R₄ is hydrocarbyl, and the other is H, or wherein R₃ and R₄ at each occurrence, is H.

21. The process according to claim 16, wherein n is 0, 1 or 2; or optionally n is 0 or 1, or optionally wherein n is 1.

22. The process according to claim 16, wherein X is H or hydrocarbyl, wherein the hydrocarbyl is alkyl, aryl or arylalkyl, or wherein the alkyl, aryl or arylalkyl is C₁₋₂₀, C₁₋₁₀ or C₁₋₈ or wherein X is H, C₁₋₁₀ alkyl, C₆₋₁₀ aryl or C₇₋₁₂ arylalkyl; or wherein X is H, C₁₋₆ alkyl, C₆₋₁₀ aryl or C₇₋₁₂ arylalkyl; or wherein X is H or phenyl, or wherein X is H.

23. The process according to claim 16, wherein R₁ and R₂ are independently H, alkyl, aryl or arylalkyl, optionally wherein the alkyl, aryl or arylalkyl is C₁₋₂₀, C₁₋₁₀ or C₁₋₆, or wherein R is H, C₁₋₁₀ alkyl, C₆₋₁₀ aryl or C₇₋₁₂ arylalkyl, or wherein R₁ and R₂ are H; and/or R₃ and R₄ are independently H, alkyl, aryl or arylalkyl, optionally wherein the alkyl, aryl or arylalkyl is C₁₋₂₀, C₁₋₁₀ or C₁₋₆, or wherein R is H, C₁₋₁₀ alkyl, C₆₋₁₀ aryl or C₇₋₁₂ arylalkyl, or wherein R₁ and R₂ are H; and/or wherein R₅ is H.

24. The process according to claim 16, wherein cleavage of at least one protecting group PG is activated by pH, temperature, radiation, or by a chemical activating agent, or by a combination thereof, optionally wherein:
the cleavage of at least one protecting group PG is activated by pH, temperature, a chemical activation agent, or by a combination thereof, and/or
at least one protecting group PG is thermally cleavable optionally in the presence of an activating agent; and/or
at least one protecting group PG is not thermally cleavable in the absence of an activating agent.

25. The process according to claim 16, wherein the activating agent is an acid or a base.

26. The process according to claim 16, wherein PG is optionally tert-butyloxycarbonyl (Boc), trityl (Trt), benzyloxycarbonyl, α,α-dimethyl-3,5-dimethoxybenzyloxycarbonyl (Ddz), 2-(4-biphenyl)isopropoxycarbonyl (Bpoc), 2-nitrophenylsulfenyl (Nps), or tosyl (Ts), or wherein the acid cleavable protecting group is selected from Boc and Trt; optionally wherein:—PG is (1,1-dioxobenzo[b]thiophene-2-yl)methyloxycarbonyl (Bsmoc), 9-fluorenylmethoxycarbonyl (Fmoc), (1,1-dioxonaphtho[1,2-b]thiophene-2-yl)methyloxycarbonyl (α-Nsmoc), 2-(4-nitrophenylsulfonyl)ethoxycarbonyl (Nsc), 2,7-di-tert-butyl-Fmoc, 2-fluoro-Fmoc, 2-monoisooctyl-Fmoc (mio-Fmoc) and 2,7-diisooctyl-Fmoc (dio-Fmoc), 2-[phenyl(methyl)sulfonio]ethyloxycarbonyl tetrafluoroborate (Pms), ethanesulfonylethoxycarbonyl (Esc), 2-(4-sulfophenylsulfonyl)ethoxycarbonyl (Sps), acetyl (Ac), benzoyl (Bz), or CF₃C(═O)— trifluoroacetamido, and optionally wherein the base cleavable protecting group is Bsmoc, Fmoc, α-Nsmoc, mio-Fmoc, dio-Fmoc, or Bsmoc; or
PG is Boc, Fmoc or Bsmoc, or
PG is Alloc.

27. The process according to claim 16, wherein at least one Y group is hydrocarbyl, or wherein at least one Y is alkyl, alkenyl, aryl, aralkyl, alkaryl, wherein said alkyl, alkenyl, aryl, aralkyl or alkaryl group is substituted with a terminal alkyne group; optionally wherein:
at least one Y group is alkyl, alkenyl, aryl, aralkyl, alkaryl, which is substituted with a terminal alkynyl group, wherein the terminal alkyne group is a C₂ to C₆ alkynyl group, or a C₂ to C₄ alkynyl group, or ethynyl; or
at least one Y group is aralkyl which is substituted with an alkynyl group or one Y group is CH₂—(C₆H₄)CH≡CH.

28. The process according to claim 1, wherein the surface comprises an electrically conductive material, or gold or silicon; and/or wherein the attachment of the nucleoside to the surface is via an association with a functionalised carbene or a functionalised alkyne, optionally via an association with a functionalised alkyne, or optionally wherein the association is with a functionalised carbene and gold, or a functionalised alkyne and silicon, and particularly wherein the association is with a functionalised alkyne and silicon.

29. The process according to claim 1, further comprising: deprotecting the oligonucleotides at the end of the oligonucleotide synthesis to form a plurality of immobilized oligonucleotides at each site, wherein the oligonucleotides are attached to the surface of a solid substrate at the 3′-position via a thermally cleavable linker group; and optionally further comprising cleavage of the thermally cleavable linker group, thereby releasing the oligonucleotide from the surface; optionally wherein the cleavage of the thermally cleavable linker group is conducted at selected sites on the surface of the solid substrate, thereby providing selective release of oligonucleotides.

30. The process according to claim 1, further comprising releasing and hybridising the oligonucleotides to form nucleic acids and releasing the nucleic acids from the surface.

31. A microarray comprising one or more nucleotides, oligonucleotides, or nucleic acids on a plurality of sites on the surface of a solid substrate, wherein the nucleotides, oligonucleotides or double stranded nucleic acids are bound to the surface by a thermally cleavable linker, wherein the thermally cleavable linker is represented by: 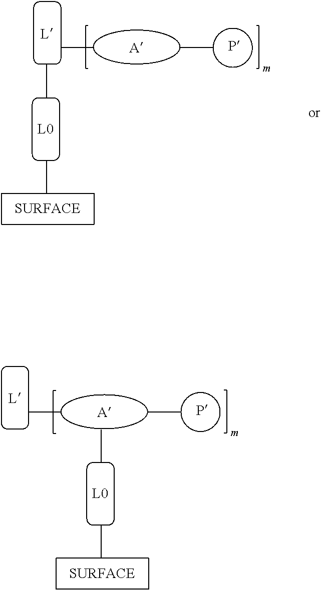
wherein:
L′-A′-P′ together is a safety catch linker attached to the surface via L0, wherein:
P′ is a protecting group,
L′ is a cleavable linker moiety,
A′ is an activator moiety that, upon removal of P′, is capable of causing cleavage of the cleavable linker group from the solid surface;
m is 1 or 2;
L0 is a moiety for attachment of the cleavable linker group to the surface.

32. A method for preparing an oligonucleotide or a nucleic acid, optionally DNA or XNA, comprising using the process of claim 1.

33. A method for preparing an oligonucleotide or a nucleic acid, optionally DNA or XNA, comprising using the microarray of claim 31.
